(19) United States
(12) Patent Application Publication
(10) Pub. No.: US 2006/0210554 A1
(43) Pub. Date: Sep. 21, 2006

(54) Detection of cholesterol ozonation products

(76) Inventors: Paul Wentworth, San Diego, CA (US); Richard A. Lerner, La Jolla, CA (US)

Correspondence Address: Schwegman, Lundberg, Woessner & Kluth, P.A., P.O. Box 2938, Minneapolis, MN, 55402, US

(21) Appl. No.: 11/369,226

(22) Filed: Mar. 6, 2006

Related U.S. Application Data
(63) Continuation of application No. PCT/US04/29063, filed on Sep. 3, 2004.
(60) Provisional application No. 60/500,593, filed on Sep. 5, 2003.
(60) Provisional application No. 60/517,821, filed on Nov. 6, 2003.

(51) Int. Cl. A61K 39/395; C07K 14/47; C07K 16/18

(52) U.S. Cl. 424/130.1; 530/402; 562/403; 564/251; 568/455; 549/266; 530/387.1

(57) ABSTRACT

The invention relates to detection of cholesterol ozonation products that are generated by atherosclerotic plaque material, and to methods of detecting vascular conditions that relate to the accumulation and oxidation of cholesterol.

### RELATED APPLICATIONS

This application is a continuation under 35 U.S.C. 111(a) of International Application No. PCT/US2004/029063 filed Sep. 3, 2004 and published in English as WO 2005/023831 A1 on Mar. 17, 2005, which claims the benefit of provisional Application Ser. No. 60/500,593, filed Sep. 5, 2003 and to provisional Application Ser. No. 60/517,821, filed Nov. 6, 2003, which applications and publication are incorporated herein in their entireties.

### STATEMENT OF GOVERNMENT RIGHTS

The invention described herein was made with United States Government support under Grant Number PO1CA 27489 awarded by the National Institutes of Health. The United States Government has certain rights in this invention.

### FIELD OF THE INVENTION

The invention relates to the discovery that cholesterol ozonation products are generated by atherosclerotic lesions. The invention provides methods for the diagnosis, detection and monitoring of patients with cholesterol related vascular conditions such as atherosclerosis and/or cardiovascular disease.

### BACKGROUND OF THE INVENTION

The population at large is continually advised that it is prudent to monitor serum cholesterol levels and is constantly reminded that an uncontrolled diet and a lack of exercise can lead to accumulation of cholesterol in arterial plaque that will increase the risk of atherosclerosis and coronary heart disease. Yet, while high serum cholesterol levels are an indicator of such risk, they are not proof that problematic atherosclerotic plaque buildup actually exists.

Serum cholesterol is known to be associated mainly with low density lipoproteins (LDL-cholesterol), high density lipoproteins (HDL-cholesterol) and the triglycerides in very low density lipoproteins (VLDL-cholesterol). Statistical evidence from a number of long term clinical tests indicates that a high proportion of HDL-cholesterol with a low proportion of LDL-cholesterol is associated with lower relative risk. HDL-cholesterol is beneficial, provided the level is not excessively low, i.e., less than 30 mg/dL. VLDL-cholesterol cholesterol has not been implicated in any risk determination, but high triglyceride itself can be a serious problem. On the other hand, a high proportion of LDL-cholesterol and a low proportion of HDL-cholesterol is an indicator of higher risk for atherosclerosis and coronary heart disease.

Even if a tight correlation exists between risk of atherosclerosis and high LDL-cholesterol levels, several studies have indicated that measurement of serum LDL- and HDL-cholesterol levels is poorly performed and often provides unreliable results. See Superko, H. R. et al. High-Density Lipoprotein Cholesterol Measurements—A Help or Hinderance in Practical Clinical Medicine, JAMA 256:2714-2717 (1986); Warnick, G. R. et al. HDL Cholesterol: Results of Interlaboratory Proficiency Test, Clin. Chem. 26:169-170 (1980); and Grundy, S. M. et al. The Place of HDL in Cholesterol Management. A Perspective from the National Cholesterol Education Program, Arch. Inter. Med. 149:505-510 (1989). The Grundy et al. article reports inter-laboratory coefficients of variance in HDL-cholesterol measurements as high as 38%. A 1987 report by the College of American Pathologists on measurement by over two thousand laboratories of the same HDL-cholesterol sample showed that more than 33% of measurements differed by more than 5% from the reference value. Inter-laboratory coefficients of variance among groups using the same method did improve to 16.5%, but such a degree of variance still indicates that most test results are too imprecise to be of any predictive value in a clinical setting. For this reason, total cholesterol:HDL-cholesterol ratios are no longer used in risk assessment.

In a typical lipid profile study, total cholesterol and triglyceride levels are measured directly from serum samples. The sample is then treated with an agent to precipitate out LDL-cholesterol and VLDL-cholesterol. HDL-cholesterol is measured in the supernatant remaining after such treatment of the sample. The VLDL-cholesterol is taken to be a fixed fraction (e.g., 0.2) of the triglyceride. LDL-cholesterol is then calculated indirectly by subtracting the values for HDL and VLDL cholesterol from the total cholesterol. The propagation of errors occurring through these three independent measurements makes the LDL-cholesterol measurement the one with the least overall accuracy and precision, although it may be the most significant for assessing cardiovascular risk. Because of such inaccuracy, it is difficult to meaningfully monitor and establish whether clinical progress has been made in LDL-cholesterol reduction therapy with time.

Thus, serum LDL-cholesterol measurements are frequently inaccurate. Such inaccuracy, coupled with the fact that LDL-cholesterol levels do not actually prove that problematic atherosclerotic lesions exist, illustrates the need for a relatively simple, reliable and reproducible method for determining whether problematic cholesterol-laden atherosclerotic lesions exist in a patient.

### SUMMARY OF THE INVENTION

According to the invention, cholesterol ozonolysis products are present in atherosclerotic plaques. Moreover, the detection and quantification of ozonation products of cholesterol in tissue and body fluids taken from a patient are accurate indicators of whether atherosclerotic lesions actually exist in the patient. The invention therefore provides simple, accurate methods for detecting whether atherosclerotic lesions exist in a patient. The methods of the invention involve detecting whether ozonation products of cholesterol are present in test samples taken from patients. The invention also contemplates quantifying the amount of cholesterol ozonation products present in biological samples as a means of diagnosing and monitoring the extent of atherosclerotic plaque formation in a mammal.

One aspect of the invention is an isolated ozonation product of cholesterol that produced in an atherosclerotic plaque. Such an ozonation product of cholesterol can, for example, have any one of formulae 4a-15a, 3c or 7c:

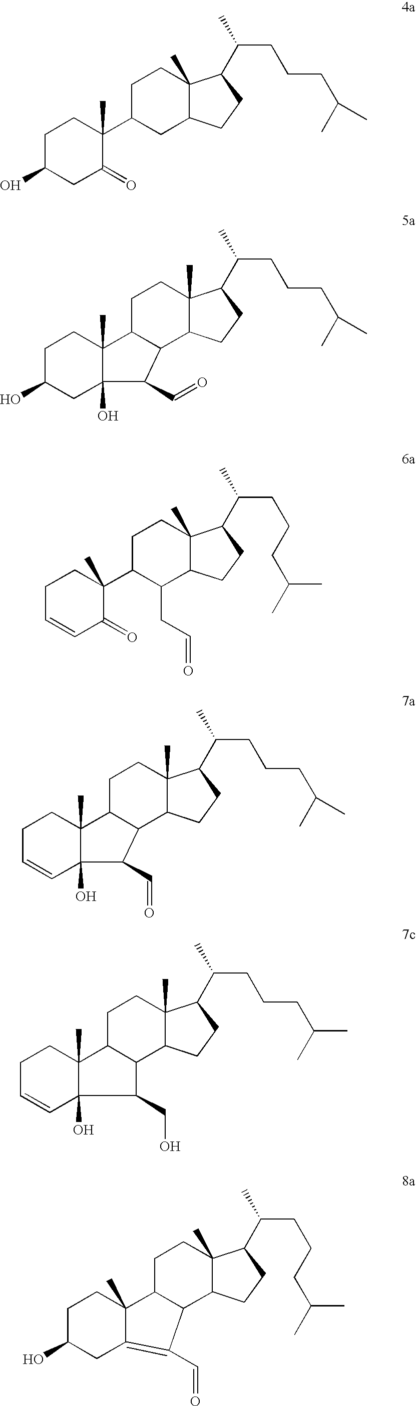

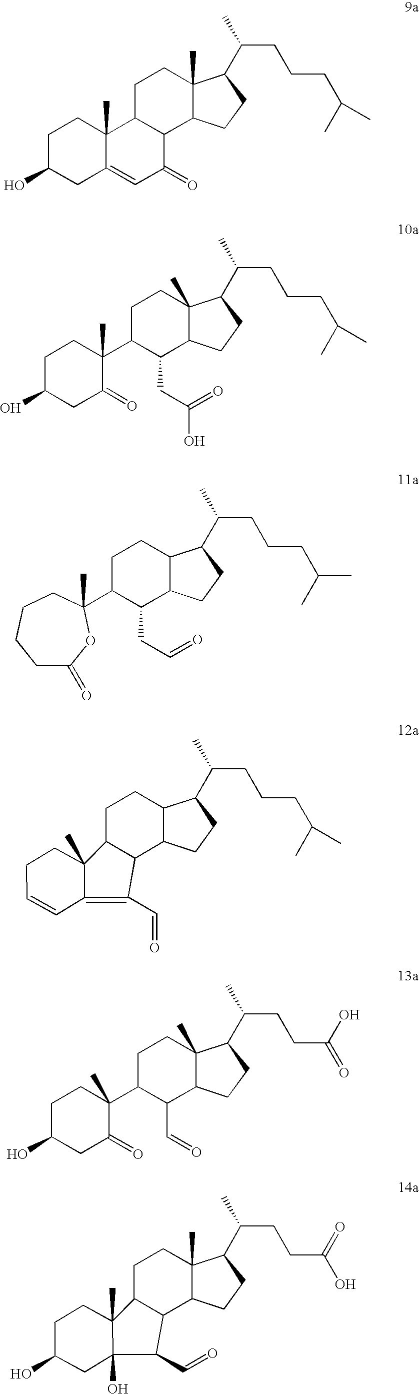

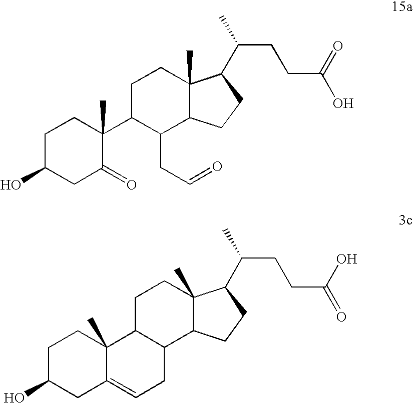

Another aspect of the invention is a detectable derivative of a cholesterol ozonation product, comprising a bisulfite adduct, an imine, an oxime, a hydrazone, a dansyl hydrazone, a semicarbazone, or a Tollins test product, wherein the ozonation product of cholesterol is generated within an atherosclerotic plaque.

Another aspect of the invention involves a hydrazone derivative of a cholesterol ozonation product that has formula 4b or formula 4c:

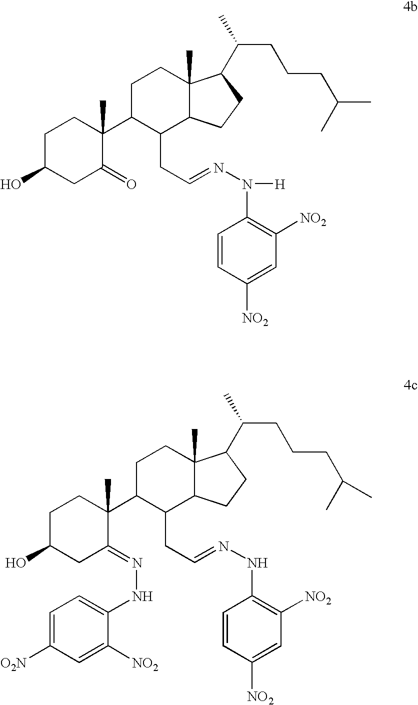

Another aspect of the invention involves a hydrazone derivative of a cholesterol ozonation product that has formula 5b:

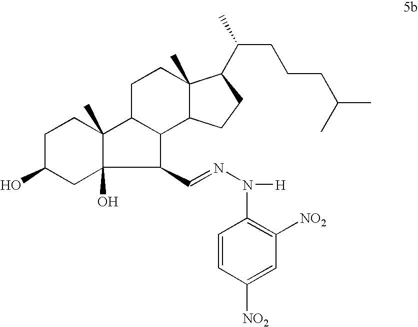

Another aspect of the invention is a hydrazone derivative of a cholesterol ozonation product that has any one of formulae 6b-15b or 10c:

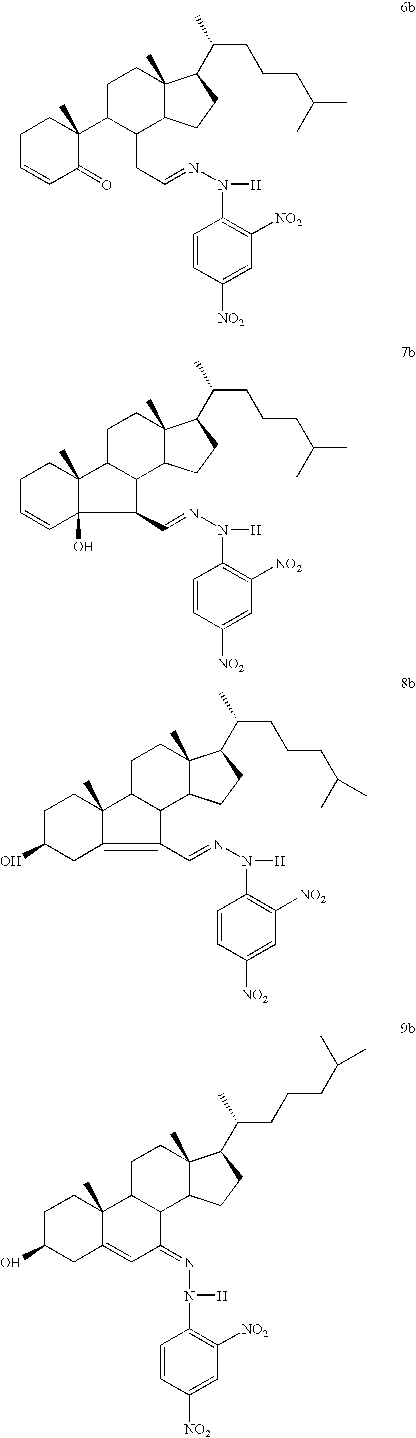

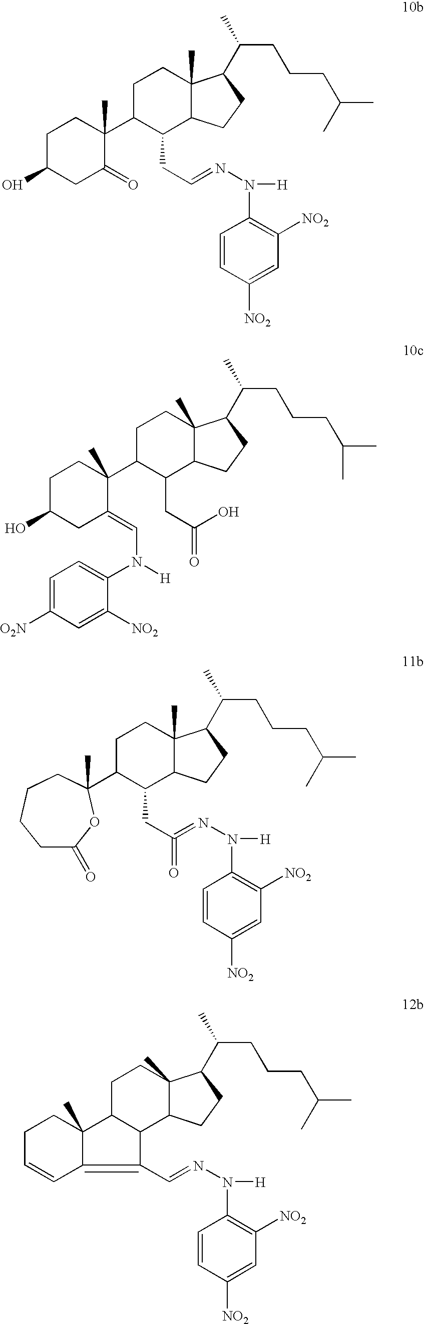

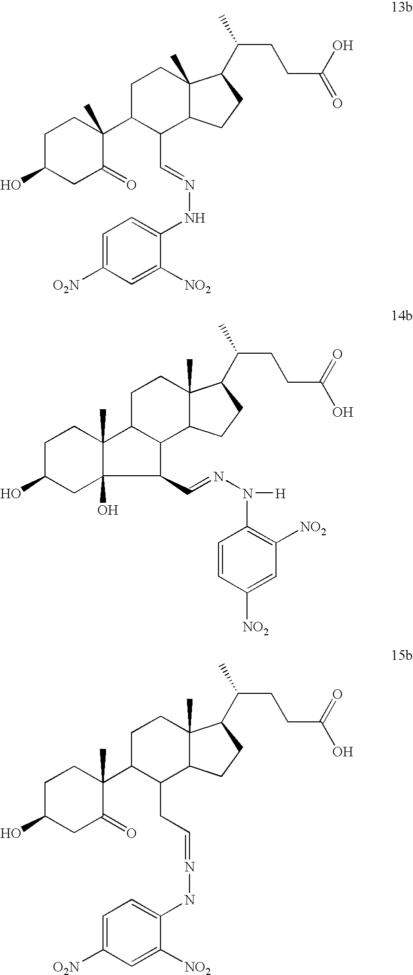

Another aspect of the invention involves a dansyl hydrazone derivative of a cholesterol ozonation product that has formula 4d:

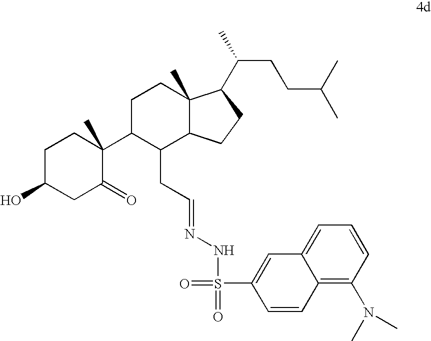

Another aspect of the invention involves a dansyl hydrazone derivative of a cholesterol ozonation product that has formula 5c:

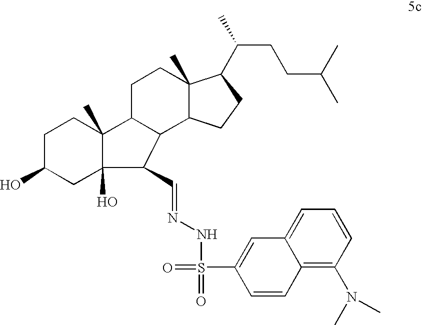

Another aspect of the invention is a hapten having formula 13a, 13b, 14a, 14b, 15a, 15c or 3c.

Another aspect of the invention is an isolated antibody that can bind to an ozonation product of cholesterol. The antibody can be a monoclonal antibody or a polyclonal antibody. The ozonation product of cholesterol to which the antibody can bind can be a compound having any one of formulae 4a-15a, 3c, 4c, 7c,. In some embodiments, the isolated antibodies that can bind to a hydrazone derivative of an ozonation product of cholesterol, for example, a compound having any one of formulae 4b-15b, 4c or 10c. Antibodies of the invention can, for example, be raised against a hapten having formula 13a, 13b, 14a, 14b, 15a, 15c or 3c.

Another aspect of the invention is an isolated antibody, wherein the isolated antibody is a derived from hybridoma KA1-11C5:6 or KA1-7A6:6 having ATCC Accession No. PTA-5427 or PTA-5428.

Another aspect of the invention is an isolated antibody, wherein the isolated antibody is a derived from hybridoma KA2-8F6:4 or KA2-1E9:4, having ATCC Accession No. PTA-5429 and PTA-5430.

Another aspect of the invention is an method for detecting atherosclerosis in a patient by detecting whether an ozonation product of cholesterol is present in the test sample obtained from a patient. The ozonation product can be generated by an atherosclerotic plaque. The test sample can, for example, be serum, plasma, blood, atherosclerotic plaque material, urine or vascular tissue. The method of detecting atherosclerosis can also involve quantifying the amount of cholesterol ozonation product that is present in the test sample.

In one embodiment, the method for detecting atherosclerosis can include a step that involved reacting the test sample with a bisulfite, ammonia, Schiff's base, aromatic or aliphatic hydrazines, dansyl hydrazine, Gerard's reagent, Tollins test reagent and detecting a derivative of an ozonation product of cholesterol that is formed by such reaction.

In another embodiment, the method for detecting atherosclerosis can include reacting the test sample with a hydrazine compound to generate a hydrazone derivative of an ozonation product of cholesterol. For example, the hydrazine compound can be 2,4-dinitrophenyl hydrazine.

In another embodiment, the method for detecting atherosclerosis can include reacting the test sample with dansyl hydrazine to generate a dansyl hydrazone derivative of an ozonation product of cholesterol. For example, the dansyl hydrazone derivative formed can have formula 4d or 5c.

In another embodiment, the method for detecting atherosclerosis can include contacting the test sample with an antibody that can bind to an ozonation product of cholesterol. Any of the antibodies described herein can be used in this method.

Another aspect of the invention involves a method for detecting whether an ozonation product of cholesterol is released by an atherosclerotic plaque in a patient by detecting whether an ozonation product of cholesterol is present in a test sample obtained from a patient, wherein the ozonation product is a compound having formula 5a. The method of detecting whether an ozonation product of cholesterol is released by an atherosclerotic plaque can also involve quantifying the amount of cholesterol ozonation product that is present in the test sample.

Another aspect of the invention involves a method for detecting atherosclerosis in a patient comprising: adding 2,4-dinitrophenylhydrazine to a test sample from the patient and detecting whether a hydrazone derivative of an ozonation product of cholesterol is present in the test sample. The hydrazone derivative detected can be a compound having any one of formulae 4b, 4c, 5b, 6b, 7b, 8b, 9b, 10b, 10c, lib, 12b, 13b, 14b or 15b.

Another aspect of the invention involves a method for detecting whether cholesterol ozonolysis products are present in a test sample by contacting macrophages with the test sample and determining whether lipid uptake by macrophages is increased.

Another aspect of the invention involves a method for detecting atherosclerosis in a patient comprising contacting macrophages with a test sample from the patient and determining whether lipid uptake by macrophages is increased.

Another aspect of the invention involves a method for detecting cholesterol ozonolysis products in a test sample comprising contacting low density lipoproteins with the test sample and observing whether the secondary structure of the low density lipoproteins changes.

Another aspect of the invention involves a method for detecting atherosclerosis in a patient comprising contacting low density lipoproteins with a test sample obtained from the patient and observing whether the secondary structure of the low density lipoproteins changes.

Another aspect of the invention involves a method for detecting cholesterol ozonolysis products in a test sample comprising contacting apoprotein B₁₀₀ with the test sample and observing whether the secondary structure of the apoprotein B₁₀₀ changes.

Another aspect of the invention involves a method for detecting atherosclerosis in a patient comprising contacting apoprotein B₁₀₀ with a test sample obtained from the patient and observing whether the secondary structure of the apoprotein B₁₀₀ changes.

The secondary structure of low density lipoproteins or apoprotein B₁₀₀ can, for example, be observed by circular dichroism.

### DESCRIPTION OF THE DRAWINGS

FIG. 1A-1D shows that indigo carmine 1 can be oxidized to form isatin sulfonic acid 2 by 4-β-phorbol 12-myristate 13-acetate (PMA)-treated human atherosclerotic lesions.

FIG. 1A illustrates the chemical changes occurring during conversion of indigo carmine 1 into isatin sulfonic acid 2 by ozone.

FIG. 1B illustrates bleaching of indigo carmine 1 by a PMA-activated atherosclerotic lesion. Each glass vial contained equal amounts of a dispersion of atherosclerotic plaque (about 50 mg wet weight) in a solution of indigo carmine 1 (200 μM) and bovine catalase (50 μg) in phosphate buffered saline (PBS, 10 mM sodium phosphate, 150 mM NaCl) pH 7.4. The photograph was taken 30 min after the addition of a solution of PMA (10 μL, 40 μg/mL) in DMSO to the vial on the right. DMSO of the same volume without PMA was added to the vial on the left. The total volume of reaction mixture was 1 mL.

FIG. 1C shows that a new HPLC peak arises in the supernatant of the +PMA vial shown in FIG. 1B, as analyzed by reversed-phase HPLC. The new peak corresponds to isatin sulfonic acid 2, having a retention time (R_{T}) of about 9.71 min.

FIG. 1D shows a negative ion electrospray mass spectrograph of a supernatant from centrifuged PMA-activated human atherosclerotic plaque material reacted with indigo carmine 1 as described above for FIG. 1B. When PMA activation of suspended plaque material was performed in H₂¹⁸O using the indicator indigo carmine 1, approximately 40% of the lactam carbonyl oxygen of indigo carmine 1 incorporated 18O, as shown by the appearance and relative intensity of the [M−H]⁻ 230 mass fragment peak in the mass spectrum of the isolated cleaved product isatin sulfonic acid 2. Isatin sulfonic acid 2 formed from indigo carmine 1 in the presence of normal water (H₂¹⁶O) has a mass fragment peak [M−H]⁻ of 228.

FIG. 2A illustrates the chemical steps involved in the ozonolysis of cholesterol 3 to give 5,6-secosterol 4a that can be converted by aldolization into 5a. Derivatization with 2,4-dinitrophenylhydrazine (2 mM in 0.08% HCl) furnished the hydrazone derivatives 4b and 5b respectively. The amount of 5b formed from 4a during the derivatization process was about 20%. The conformational assignments of 5a and 5b were assigned as described by K. Wang, E. Bermúdez, W. A. Pryor, Steroids 58, 225 (1993).

FIG. 2B shows the structures of oxysterols 6a-9a and 2,4-dinitrophenylhydrazine hydrochloride derivatives 6b-7b investigated as standards for the peak eluting at about 18 min [M−H]⁻ 579 in FIG. 3. The conformational assignments of 7a-7b were based on a ¹H—¹H ROESY experiment using authentic synthetic 7b material.

FIG. 3A-E illustrate an analysis of plaque material and chemically synthesized authentic samples of hydrazones 4b, 5b and 6b using liquid chromatography mass spectroscopy (LCMS). Conditions: Adsorbosphere-HS RP-C18 column, 75% acetonitrile, 20% water, 5% methanol, 0.5 mL/min flow rate, 360 nm detection, in-line negative ion electrospray mass spectrometry (MS) (Hitachi M8000 machine) of a plaque extract after derivatization with 2,4-dinitrophenylhydrazine hydrochloride (DNPH HCl).

FIG. 3A illustrates an LCMS analysis of a plaque material without PMA activation but after derivatization with 2,4-dinitrophenylhydrazine as described herein. Compounds 4b (RT˜14.1 min), 5b (RT˜20.5 min) and 6b (RT˜18 min) were detected in an atherosclerotic lesion before activation with PMA (40 μg/mL).

FIG. 3B illustrates an LCMS analysis of plaque material after activation with PMA (40 μg/mL), extraction and derivatization with 2,4-dinitrophenylhydrazine as described above. Larger amounts of compound 4b (RT˜14.1 min), but smaller amounts of compound 6b (RT˜18 min) were detected in an atherosclerotic lesion after activation with PMA (40 μg/mL).

FIG. 3C illustrates an HPLC analysis of authentic 4b; the inset shows the mass spectroscopy analysis.

FIG. 3D illustrates an HPLC analysis of authentic 6b; the inset shows the mass spectroscopy analysis.

FIG. 3E illustrates an HPLC analysis of authentic 5b; the inset shows the mass spectroscopy analysis.

FIG. 4A-D illustrate HPLC-MS analyses of extracted and derivatized atherosclerotic material where a 100 μl injection volume was used to allow detection of trace hydrazones. FIG. 4A shows a LC trace of time versus intensity using the conditions detailed vide supra. R_{T} 26.7 is 7b (by comparison to authentic material). The peak at R_{T}˜24.7 is an unknown hydrazone with [M−H]⁻ 461. FIG. 4B provides a single ion monitoring of [M−H]⁻ 597. FIG. 4C provides a single ion monitoring of [M−H]⁻ 579. FIG. 4D shows a single ion monitoring of [M−H]⁻ 461.

FIG. 5A-C illustrates the concentrations of cholesterol ozonation products in atherosclerotic extracts for patients A-N.

FIG. 5A is a bar chart showing the measured concentration of hydrazone 4b after extraction and derivatization of 4a from atherosclerotic lesions of patients, pre- and post-activation with PMA. The bar chart shows the numerical values of the amounts detected before and after activation as determined by a Student t-test (two-tail) (p<0.05, n=14) analysis using GraphPad Prism V3 for Macintosh.

FIG. 5B is a bar chart showing the measured concentration of 5b after extraction and derivatization of 5a from atherosclerotic lesions of patients, pre- and post-activation with PMA (n=14).

FIG. 5C is a bar chart showing measured concentrations of 5b after extraction and derivatization of 5a from plasma samples taken from patients. Cohort A (n=8) patients were to undergo a carotid endarterectomy procedure within 24 h (plasma analysis was performed 3 days after sample collection). Cohort B (n=15) patients were randomly selected from patients attending a general medical clinic (plasma analysis was performed 7 days after sample collection). Note that in a preliminary investigation plasma levels of 5a, fall by about 5% per day. Under the conditions of this assay, the detection limit of 4b and 5b was 1-10 nM. Therefore, in cases where no 4b or 5b was apparent, the level of 4b or 5b was less than 10 nM.

FIG. 6A illustrates the cytotoxicity of 3, 4a and 5a against B-cell (WI-L2) cell line. Each data point is the mean of at least duplicate measurements. The IC₅₀s±standard errors for 4a (▪) and 5a (▴) were calculated using non-linear regression analysis (Hill plot analysis), with GraphPad Prism v 3.0 for the Macintosh computer. No cytotoxicity with 3 (▾) was observed in this concentration range.

FIG. 6B illustrates the cytotoxicity of 3, 4a and 5a against T-cell (Jurkat) cell line. Each data point is the mean of at least duplicate measurements. The IC₅₀s±standard errors for 4a (▪) and 5a (▴) were calculated using non-linear regression analysis (Hill plot analysis), with GraphPad Prism v 3.0 for the Macintosh computer. No cytotoxicity with 3 (▾) was observed in this concentration range.

FIG. 7A-B shows that of cholesterol ozonolysis products 4a and 5a increase lipid-loading by macrophages to produce foam cells.

FIG. 7A shows that LDL incubated with J774.1 macrophages has little effect upon lipid-loading of those macrophages. Macrophages were first grown for 24 h in RPMI-1640 containing 10% fetal bovine serum and then incubated for 72 h in the same media containing LDL (100 μg/mL). Cells were fixed with 4% formaldehyde and stained with hematoxylin and oil red O such that lipid granules stained a darker red color. Magnification×100.

FIG. 7B shows that LDL incubated with ozonolysis product 4a induces lipid-loading of macrophages to produce foam cells. J774.1 macrophages were grown for 24 h in RPMI-1640 containing 10% fetal bovine serum. Cells were then incubated for 72 h in the same media containing LDL (100 μg/mL) and ozonolysis product 4a (20 μM). Cells were fixed with 4% formaldehyde and stained with hematoxylin and oil red O such that lipid granules stained a darker red color. Magnification×100. Note that the effect of ozonolysis product 4a upon macrophages was indistinguishable from the effect of ozonolysis product 5a.

FIG. 8A-C shows that the secondary structure of proteins in LDL is altered by exposure to ozonolysis product 4a or 5a, as detected by circular dichroism. Results reported are from at least duplicate experiments for each sample.

FIG. 8A shows that the protein content of normal LDL has a large proportion of α helical structure (˜40±2%) and smaller amounts of β structure (˜13±3%), β turn (˜20±3%) and random coil (27±2%). FIG. 8A shows time-dependent circular dichroism spectra of LDL (100 μg/ml) at 37° C. in PBS (pH 7.4).

FIG. 8B shows that incubation of LDL with ozonolysis product 4a in PBS (pH 7.4) at 37° C. leads to a loss of secondary structure of apoB-100. FIG. 8A shows time-dependent circular dichroism spectra of LDL (100 μg/ml) and 4a (10 μM) at 37° C. in PBS (pH 7.4).

FIG. 8C shows that incubation of LDL with ozonolysis product 5a in PBS (pH 7.4) at 37° C. leads to a loss of secondary structure of apoB-100. FIG. 8A shows time-dependent circular dichroism spectra of LDL (100 μg/ml) and 5a (10 μM) at 37° C. in PBS (pH 7.4).

FIG. 9 illustrates the structures for dansyl hydrazine cholesterol ozonation products 4a and 5a (4d and 5c, respectively) and the HPLC elution patterns of these hydrazine derivatives. As shown, cholesterol ozonation products 4a and 5a give rise to dansyl hydrazone conjugates having different HPLC retention times.

FIG. 10 illustrates that cholesterol ozonation products can be detected in human carotid artery specimens by gas chromatography-mass spectroscopy (GCMS) analysis. The chromatogram shown is typical of atherosclerotic plaque extracts. The peak eluting at 22.49 minutes is the peak corresponding to both cholesterol ozonation products 4a and 5a. The insert mass spectrometry chromatograph illustrates that the species eluting at 22.49 minutes has m/z 354.

FIG. 11 provides a quantitative analysis of two atherosclerotic plaques (P1 and P2) by ID-GCMS. The amounts of cholesterol ozonation products 4a and 5a detected were about 80-100 pmol/mg tissue and were similar to those detected by LC-MS analysis. Each bar represents a duplicate extract and is reported as the mean±SEM.

### DETAILED DESCRIPTION OF THE INVENTION

The invention provides methods for detecting ozonation products of cholesterol. Also provided are kits and reagents for detecting ozonation products of cholesterol. These methods, kits and reagents are useful for detecting vascular conditions that are related to cholesterol build up. For example, the methods, kits and reagents are useful for diagnosing and monitoring the prognosis of inflammatory artery diseases such as atherosclerosis.

Cholesterol Ozonation

According to the invention, cholesterol is oxidized within atherosclerotic arteries by reactive oxygen species such as ozone. A number of cholesterol ozonation products are generated by this process and can be detected in tissue or fluid samples taken from patients suffering from atherosclerosis. Detection of cholesterol ozonation products is diagnostic of inflammatory artery disease such as atherosclerosis.

Cholesterol has the following structure (3). 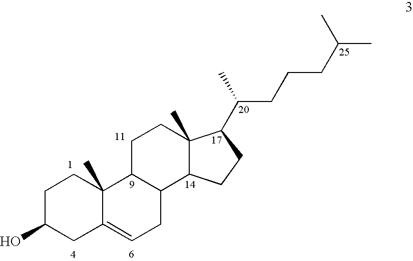
While high levels of cholesterol in the blood are correlated with a likelihood for forming atherosclerotic plaques, such high levels of cholesterol do not definitively indicate that atherosclerotic plaques are present in the arterial system of a patient. To ascertain whether a patient actually has atherosclerotic lesions, expensive testing is now used such as rapid CAT scans, dye injections with imaging procedures, or invasive endoscopic or catheterization procedures.

However, according to the invention, the existence of actual atherosclerotic plaques can be detected by detecting the ozonation products of cholesterol. When cholesterol is laid down in an artery an atherosclerotic plaque can form. While not wishing to be limited to a specific mechanism, it appears that macrophages, neutrophils, and other immune cells become enmeshed within the atherosclerotic lesion and release reactive oxygen species such as ozone. The reactive oxygen species produced react with the cholesterol in the lesion and oxidize the cholesterol into a number of products that can be detected in the patient. Hence, two events occur in order for cholesterol ozonation products to appear in samples taken from the patient. First, there must be substantial buildup of cholesterol within atherosclerotic plaque. Second, the atherosclerosis must have progressed to the stage where reactive oxygen species are produced. It is the juxtapositioning of these two events that leads to formation of cholesterol ozonation products. Because cholesterol buildup and ozone production occur in substantially no other situation, detection of cholesterol ozonation products is an accurate indicator of whether inflammatory artery conditions such as atherosclerosis exist in a patient. Moreover, according to the invention, the amount of cholesterol ozonation product(s) present within biological samples (e.g. serum) taken from patients suffering from atherosclerosis is an indicator of the severity of the arthrosclerosis suffered by the patient.

According to the invention, have identified a number of cholesterol ozonation products. For example, when cholesterol 3 is oxidized, the seco-ketoaldehyde 4a and its aldol adduct 5a are the main products formed.

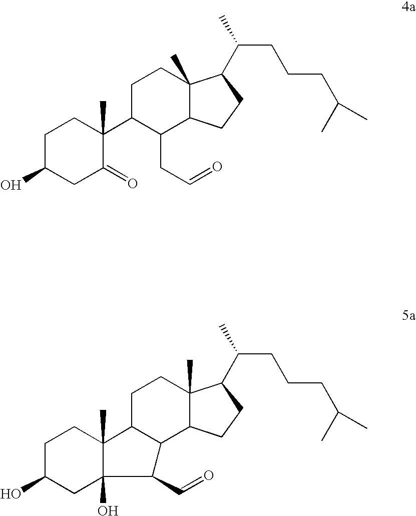

In addition, cholesterol ozonation products having structures like those of compounds 6a-15a, and 7c are also observed.

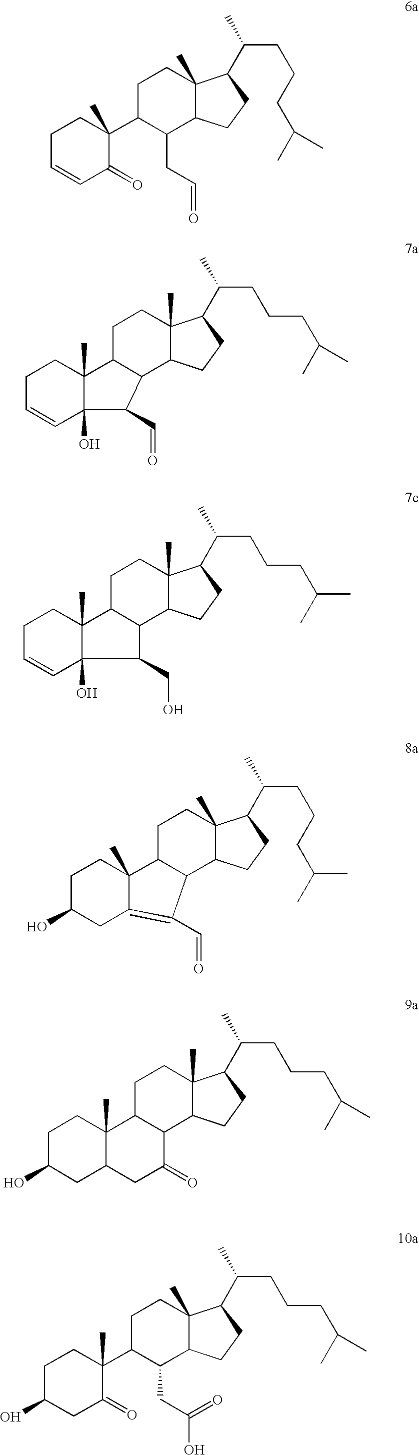

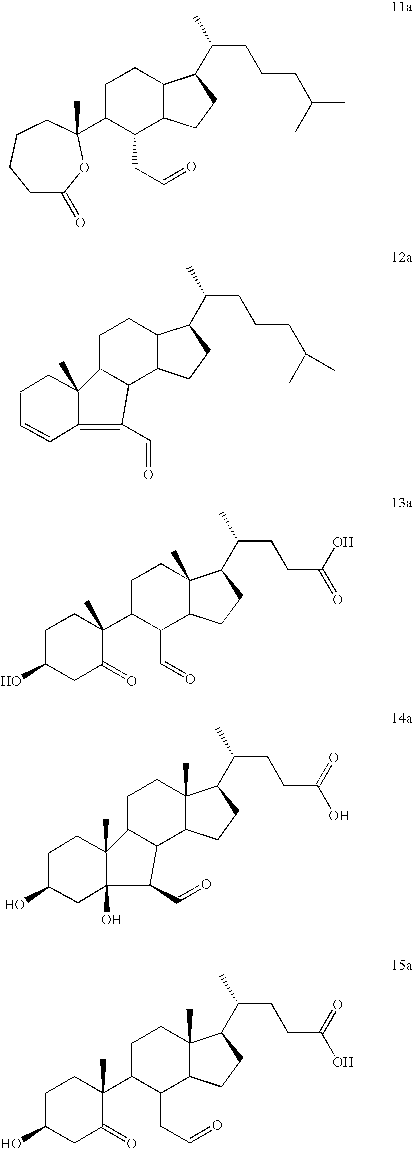

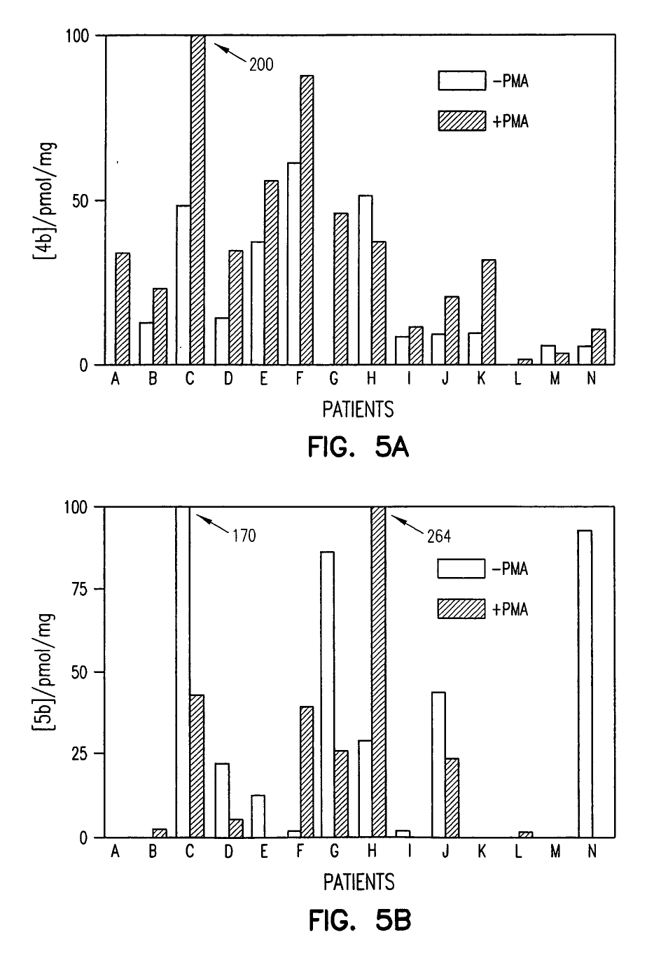

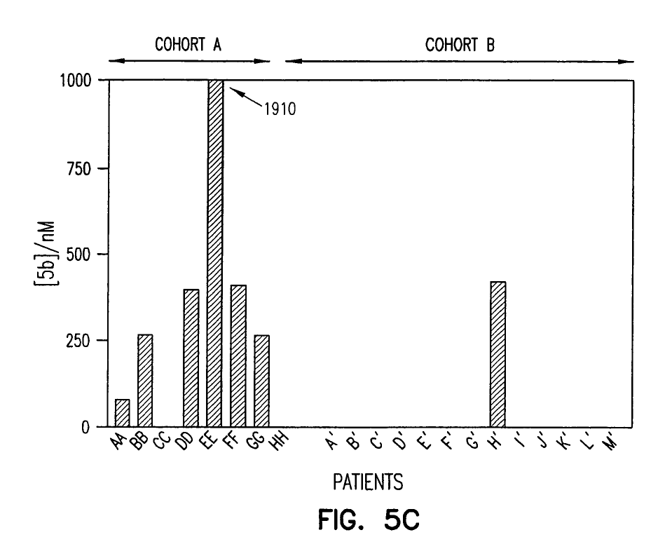

According to the invention, the seco-ketoaldehyde 4a, its aldol adduct 5a and the related compounds 6a-15a and 7c can be present in atherosclerotic plaques and in the bloodstream of patients suffering from atherosclerosis. Moreover, the amount of the seco-ketoaldehyde 4a, its aldol adduct 5a and the related compounds 6a-15a and 7c is correlated with the extent and severity of atherosclerotic plaque formation in the patient. For example, in six of eight patients with atherosclerosis disease states that were sufficiently advanced to warrant endarterectomy the aldol 5a was detected, in amounts ranging from 70-1690 nM (FIG. 5C). However, in only one of fifteen plasma samples from patients that were randomly selected from a group of patients attending a general medical clinic was there detectable 5a.

The invention therefore contemplates detection of these cholesterol ozonation products for determining whether a patient has atherosclerotic lesions and for determining the extent to which the circulating cholesterol has become incorporated into atherosclerotic plaques.

Detection of Ozone and Cholesterol Products

Cholesterol ozonation products can be detected or identified by any procedure available to one of skill in the art. For example, these products can be detected or identified by high pressure liquid chromatography (HPLC), by liquid chromatography mass spectroscopy (LCMS), by gas chromatography (GC), by gas chromatography mass spectroscopy (GCMS), by high pressure liquid chromatography mass spectroscopy (HPLC-MS), by HPLC with evaporative light scattering detection (ELSD), by ion detection with gas chromatography mass spectroscopy (ID-GCMS), by visible, ultraviolet or infrared spectroscopy, by thin layer chromatography, by electrophoresis, by liquid chromatography, by nuclear magnetic resonance, by wet chemical assay, by immunoassay (e.g. ELISA), by immunohistochemistry, fluorescence spectroscopy, light spectroscopy or ultraviolet spectroscopy or by any other means available to one of skill in the art.

Moreover, the presence of cholesterol ozonation products can also be detected by observing the effects of that these products have upon low density lipoproteins (LDLs), apoprotein B₁₀₀ (apoB-100, the protein component of LDL), or macrophages. As described herein, cholesterol ozonolysis products 4a and 5a can promote formation of foam cells from macrophages. Moreover, cholesterol ozonolysis products 4a and 5a modify the secondary structures of LDL and apoB-100. Hence, the presence of cholesterol ozonolysis products in test samples can be detected by determining whether the test samples can promote foam cell formation or alter the secondary structure of LDLs or apoprotein B₁₀₀. These assays are described in greater detail below.

In some embodiments, test samples are reacted with a reagent that facilitates detection and identification of cholesterol ozonation products. For example, test samples can be contacted with any fluorescent, phosphorescent or colored reagent that reacts with a cholesterol ozonation product and the product of the reaction can be detected using a fluorescence, visible or ultraviolet light detector. In other embodiments, no such reagent is employed and the cholesterol ozonation products are identified by their physical or chemical properties. Such methods are described in more detail below.

The amount of ozone in atherosclerotic plaque materials is also indicative of the amount of atherosclerotic plaque that has formed. Hence, the invention contemplates detection and/or quantification of ozone in atherosclerotic plaque material to assess the size of an atherosclerotic plaque. Ozone can be detected in atherosclerotic plaque material by use of any reagent that can detect ozone. For example, indigo carmine 1 is a colored reagent whose blue color is lost upon reaction with ozone. In the process, isatin sulfonic acid 2 formed as shown below. 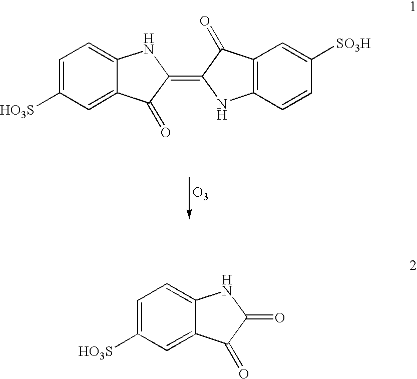
Hence, ozone detection methods can be used to evaluate the extent of atherosclerotic plaque build-up.

However, while ozone can be detected in atherosclerotic material, cholesterol ozonation products can be detected in the bloodstream of a patients having substantial atherosclerotic plaque material. Hence, to avoid isolation of atherosclerotic plaque material, one of skill in the art may choose to isolate a blood sample and then detect whether ozonation products of cholesterol are present. This avoids expensive, intrusive procedures such as endarterectomy and provides a reliable procedure for assessing how much atherosclerotic plaque material is present in the patient.

To diagnose atherosclerosis, any of the cholesterol ozonation products, for example, the seco-ketoaldehyde 4a, its aldol adduct 5a and/or the related compounds 6a-15a and 7c can be detected. However, studies performed to date indicate that the aldol adduct 5a is one of the main products that can be detected in serum.

In some embodiments, the cholesterol ozonation products obtained in biological samples can be chemically modified to facilitate detection. Reagents that can be used for such chemical modification include bisulfites, ammonia, Schiff's bases (using aliphatic or aromatic amine such as aniline), aromatic or aliphatic hydrazines, dansyl hydrazines, Gerard's reagent (semicarbazides), Tollins test reagents (formaldehyde and calcium hydroxide) and the like. When reacted with the cholesterol ozonation products of the invention, these reagents provide distinctive products such as bisulfite adducts (readily crystallized as sodium salts), imines, oximes, hydrazones, semicarbazones, Tollins test products, and the like that can readily be detected by one of skill in the art.

For example, hydrazone derivatives of the seco-ketoaldehyde 4a, its aldol adduct 5a or the related compounds 4c, 6a-15a and 7c can be readily formed and are useful markers for determining whether a patient has atherosclerotic lesions. These hydrozone derivatives include compounds having structures like those of compounds 4b-15b, and possibly 4c or 10c.

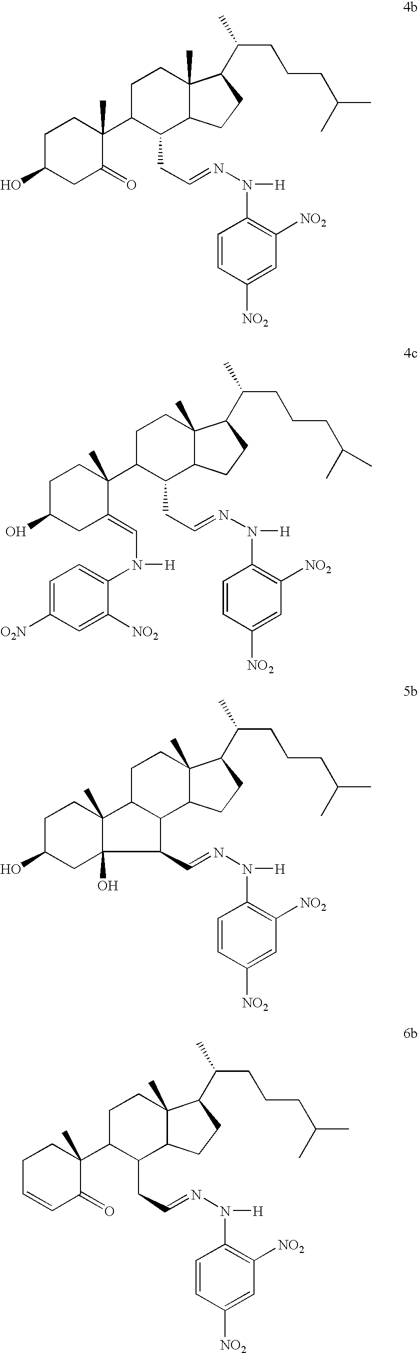

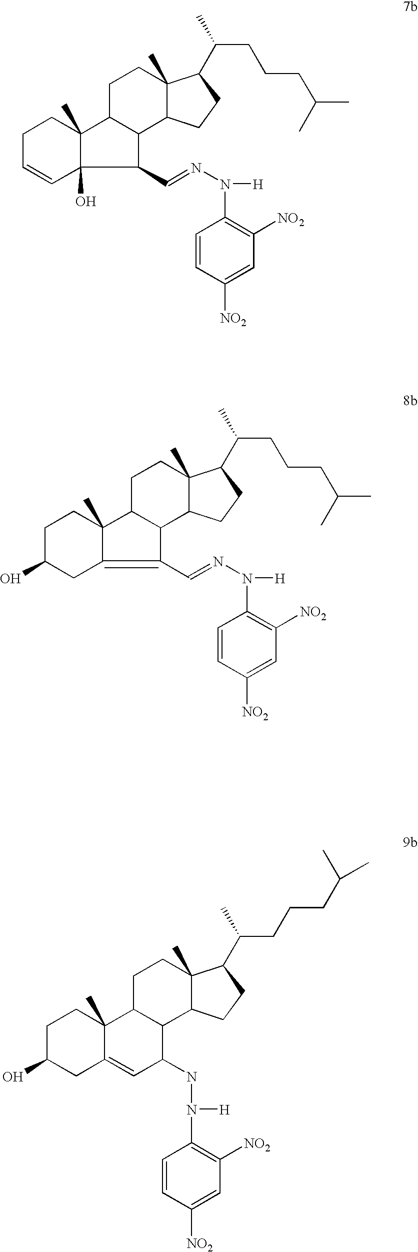

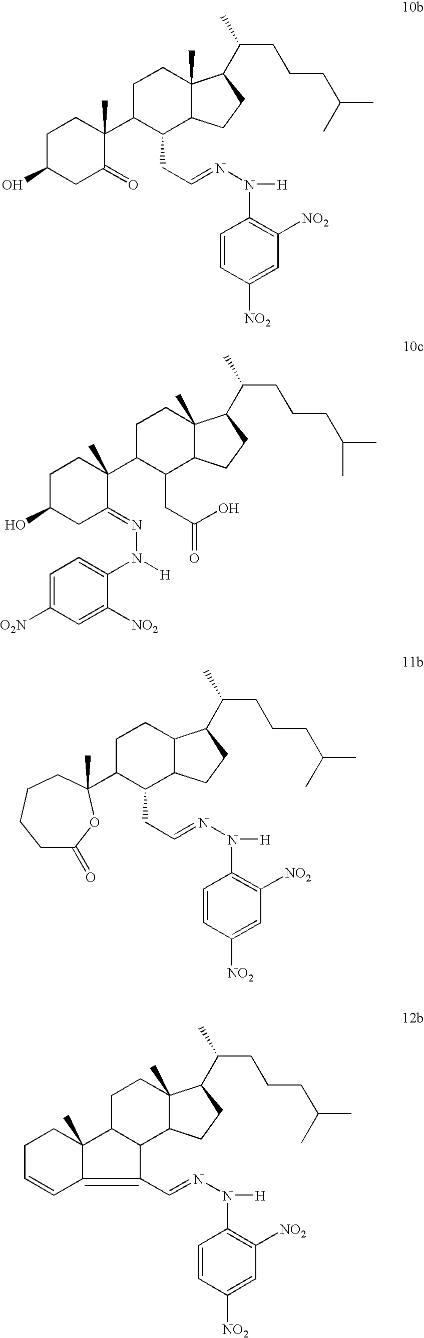

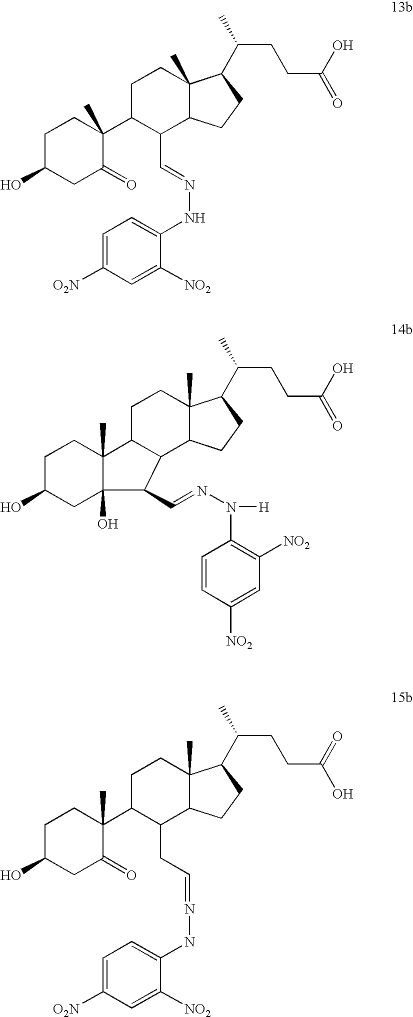

These hydrozone derivatives have been detected using HPLC mass spectroscopy in concentrations as low as about 1 nM to 10 nM. Using gas chromatography mass spectroscopy analysis, as little as 10 fg/μL of the cholesterol ozonation products can be detected.

Cholesterol ozonation products can be converted to hydrozone derivatives, for example, by reaction with a hydrazine compound such as 2,4-dinitrophenyl hydrazine. In some embodiments, the reaction is carried out in an organic solvent such as acetonitrile, or alcohol (e.g. methanol or ethanol). An acidic environment and a non-oxygen containing, non-reactive atmosphere are often utilized.

For example, plasma can be obtained from a patient and placed in EDTA. This sample can be washed several times with dichloromethane to extract the cholesterol ozonation products. The dichloromethane fractions can be evaporated in vacuo and the residue containing the cholesterol ozonation products can be dissolved in alcohol (e.g. methanol). A solution of 2,4-dinitrophenyl hydrazine and 1N HCl in ethanol can then be added. Nitrogen can be bubbled through the solution for a short time (e.g. 5 min) to remove free oxygen. The solution can be stirred for a time sufficient for converting the cholesterol ozonation products to their hydrazone derivatives (e.g. 2 h). The major product detected in this procedure is believed to be the hydrazone derivative of the aldol adduct 5a. Moreover, preliminary investigations have revealed that the amount of 5a that can be extracted from plasma decreases by about 5% per day. Hence, fresh plasma samples will give more accurate measurements of the actual amount of the aldol adduct 5a in a sample.

The reagents and methods of the invention can be utilized to detect atherosclerosis at any stage in its progression. According to the new classification adopted by the AHA and used for this study, eight lesion types can be distinguished during progression of atherosclerosis.

Type I lesions are formed by small lipid deposits (intracellular and in macrophage foam cells) in the intima and cause very initial and the most minimal changes in the arterial wall. Such changes do not thicken the arterial wall.

Type II lesions are characterized by fatty streaks that are yellow-colored streaks or patches that increase the thickness of the intima by less than a millimeter. They consist of accumulation of more lipid than is observed in type I lesions. The lipid content is approximately 20-25% of the dry weight of the lesion. Most of the lipid is intracellular, mainly in macrophage foam cells, and smooth muscle cells. The extracellular space may contain lipid droplets, but these are smaller than those within the cell, and small vesicular particles. Chemically, the lipid consists of cholesterol esters (cholesteryl oleate and cholesteryl linoleate), cholesterol, and phospholipids.

Type III lesions are also described as preatheroma lesions. In type III lesions the intima is thickened only slightly more than observed for type II lesions. Type III lesions do not obstruct arterial blood flow. The extracellular lipid and vesicular particles are identical to those found in type II lesions, but are present in increased amount (approximately 25-35% dry weight) and start to accumulate in small pools.

Type IV lesions are associated with atheroma. They are crescent-shaped and increase the thickness of the artery. The lesion may not narrow the arterial lumen much except for persons with very high plasma cholesterol levels (for many people, the lesion can not be visible by angiography). Type IV lesions consist of an extensive accumulation (approx. 60% dry weight) of extracellular lipid in the intimal layer (sometimes called a lipid core). The lipid core may contain small clamps of minerals. These lesions are susceptible to rupture and to formation of mural thrombi.

Type V lesions are associated with fibroatheroma. They have one or multiple layers of fibrous tissue consisting mainly of type I collagen. Type V lesions have increased wall thickness and, as the atherosclerosis progresses increased reduction of the lumen. These lesions have features that permit further subdivision. In type Va lesions, the new tissue is part of a lesion with a lipid core. In type Vb lesions, the lipid core and other parts of the lesion are calcified (leading to Type VII lesions). In type Vc lesions, the lipid core is absent and lipid generally is minimal (leading to Type VIII lesions). Generally, the lesions that undergo disruption are type Va lesions. They are relatively soft and have a high concentration of cholesterol esters rather than free cholesterol monohydrate crystals. Type V lesions can rupture and form mural thrombi.

Type VI lesions are complicated lesions having disruptions of the lesion surface such as fissures, erosions or ulcerations (Type VIa), hematoma or hemorrhage (Type VIb), and thrombotic deposits (Type VIc) that are superimposed on Type IV and V lesions. Type VI lesions have increased lesion thickness and the lumen is often completely blocked. These lesions can convert to type V lesions, but they are larger and more obstructive.

Type VII lesions are calcified lesions characterized by large mineralization of the more advanced lesions. Mineralization takes the form of calcium phosphate and apatite, replacing the accumulated remnants of dead cells and extracellular lipid.

Type VIII lesions are fibrotic lesions consisting mainly of layers of collagen, with little lipid. Type VIII could be a consequence of lipid regression of a thrombus or of a lipidic lesion with an extension converted to collagen. These lesions may obstruct the lumen of medium-sized arteries.

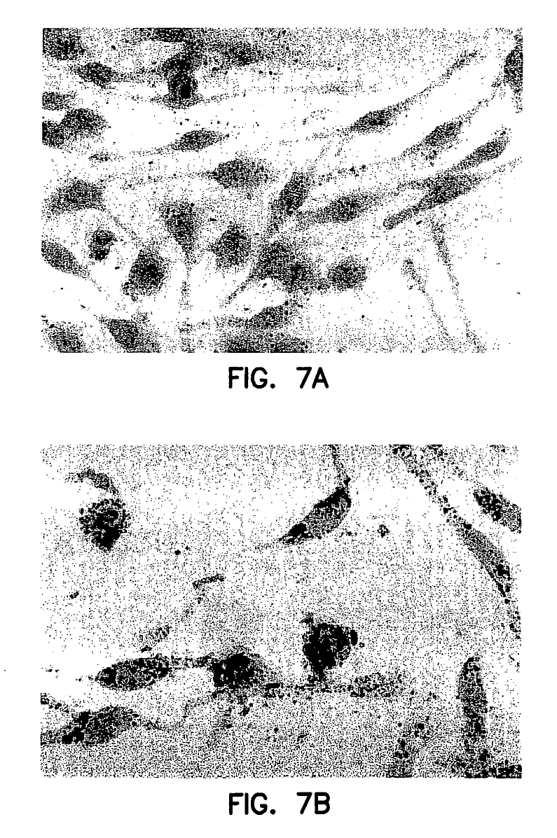

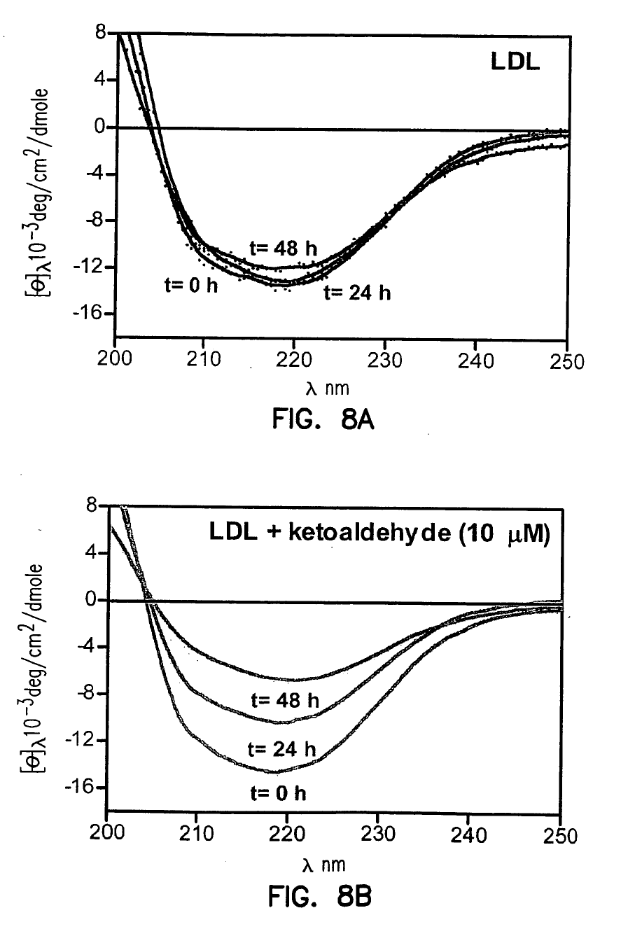

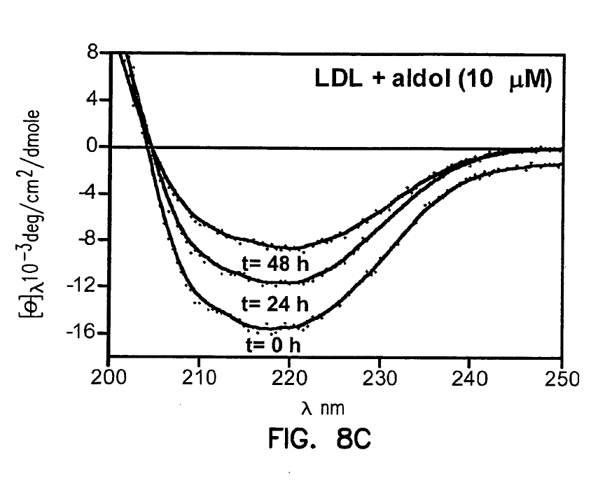

As described herein, cholesterol ozonolysis products 4a and 5a can promote formation of foam cells from macrophages and modify the structure of low density lipoproteins (LDLs) and apoprotein B₁₀₀, the protein component of LDL. LDL was incubated with 4a or 5a in the presence of unactivated murine macrophages. After exposure to 4a or 5a these macrophages began lipid-loading and foam cells began to appear in the reaction vessel (see FIG. 7). Moreover, incubation of human LDL (100 μg/ml) with 4a and 5a (10 μM) led to time-dependent changes in the structure of apoB-100 as detected by circular dichroism (FIGS. 8B,C). As shown in FIG. 8A, the protein content of normal LDL has a large proportion of a helical structure (˜40±2%) and smaller amounts of P structure (˜13±3%), β turn (˜20±3%) and random coil (27±2%). However, when LDL is incubated with 4a and 5a, there is a significant loss of secondary structure. The loss of secondary structure is mainly a loss of a helical structure (4a˜23±5%; 5a˜20±2%). A correspondingly higher percentage of random coil is observed (4a˜39±2%; 5a 32±4%). Hence, the 4a and 5a cholesterol ozonolysis products may directly lead to some of the physiological changes associated with problematic atherosclerosis.

The invention therefore provides methods for diagnosing whether problematic cholesterol ozonolysis products are present in test samples. In some embodiments, such methods involve determining whether the teat samples can cause changes in lipid uptake by macrophages. If increased lipid uptake is observed after incubating a test sample with macrophages, then the test sample has cholesterol ozonolysis products and the patient from whom the test sample was obtained likely has problematic atherosclerosis. In another embodiment, the invention provides methods for detecting cholesterol ozonolysis products in a test sample by detecting whether the test sample can modify the secondary structure of LDL or apoprotein B₁₀₀. The secondary structure of LDL or apoprotein B₁₀₀ can be monitored or observed using methods available to one of skill in the art, for example, circular dichroism or calorimetry.

Quantitative measurements of the cholesterol ozonation products in biological samples can be used to diagnose which atherosclerosis stage and/or what types of lesions are present in the animal from which the biological samples were obtained. Biological samples from populations of patients known to have distinct types of lesions or distinct stages of atherosclerosis are tested and the amount of cholesterol ozonation products in these samples can be tabulated. Such tabulation permits statistical analysis and correlation between the atherosclerosis stage (or lesion type) and the amount of cholesterol ozonation product in a patient's sample. Mean values and ranges of amounts of cholesterol ozonation products can be calculated for each population of patients so that knowledge of the amount of cholesterol ozonation product in a new patient's sample permits prediction of the stage of atherosclerosis existing in the new patient. Similarly, the degree to which biological samples can cause lipid loading by macrophages or changes in the secondary structures of low density lipoproteins and/or apoprotein B₁₀₀ can also be quantified and correlated with the stage of atherosclerosis and/or the types of lesions present in atherosclerotic patients.

Quantitative measurements of the amounts of cholesterol ozonation product in patients' samples can be by any available method. For example, quantitative measurements can be made by determining the area under the peak of readouts from high pressure liquid chromatography (HPLC), liquid chromatography mass spectroscopy (LCMS), visible spectroscopy, ultraviolet spectroscopy, infrared spectroscopy, gas chromatography, liquid chromatography, or other means available to one of skill in the art. In other embodiments, the size or optical density of a thin layer chromatography spot or electrophoretic band can be used to quantify the amount of cholesterol ozonation product in a sample. The optical density of a wet chemical reaction assay mixture, color reaction or of an immunoassay (e.g. ELISA) can also be used to quantify the amount of cholesterol ozonation product in a sample. The percent or number of macrophages that exhibit lipid loading upon exposure to a test sample can also be used as a quantitative measurement of the amount of cholesterol ozonolysis product in test samples. Similarly, the extent or percent of change in LDL or apoprotein B₁₀₀ secondary structure upon exposure to a test sample can be used as a quantitative measurement of the amount of cholesterol ozonolysis product in test samples.

In another embodiment, such products can be detected by immunoassay. The invention provides antibodies and binding entities that can bind any of the compounds of formulae 3, 4a-15a, 4b-15b, 3c, 4c, 7c or 10c. The invention is further directed against haptens that are structurally related to the cholesterol ozonation products and the hydrazone derivatives of such ozonation products. For example, the invention provides a hapten having formula 3c, 13a, 13b, 14a, 14b, 15a or 15b that can be used to generate antibodies that can react with the ozonation and hydrazone products of cholesterol: 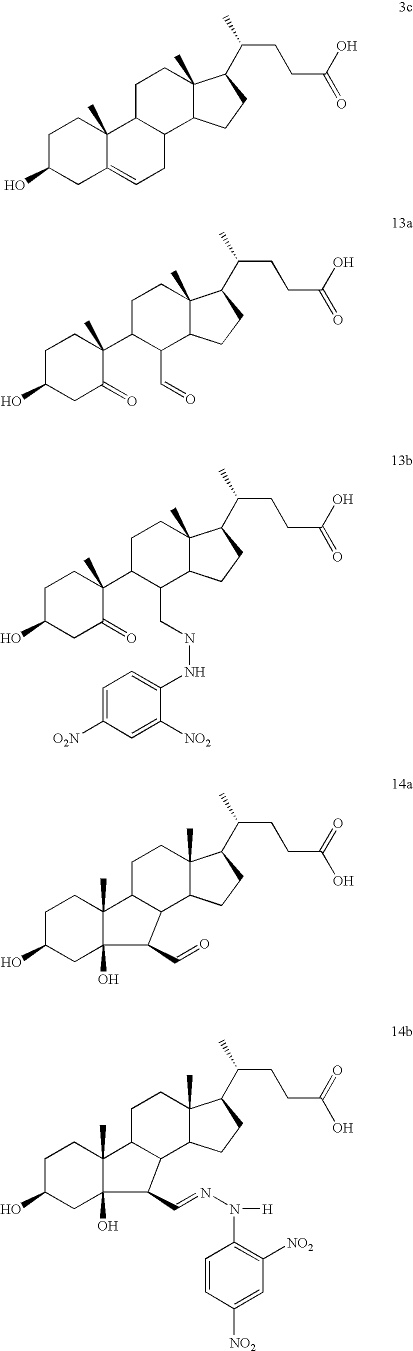 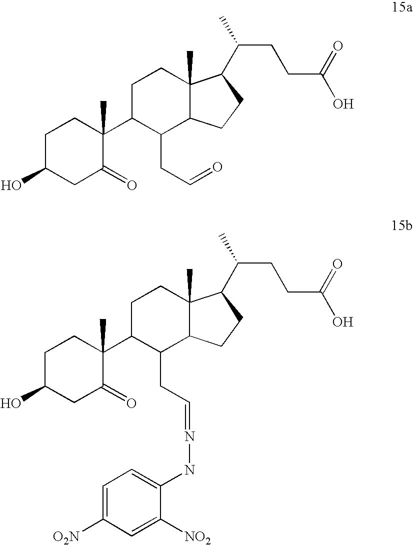
Antibodies and Binding Entities

The invention provides antibody preparations and binding entities directed against cholesterol ozonation products, haptens and related cholesterol-like molecules that are useful for detecting and identifying cholesterol ozonation products. For example, the antibodies or binding entities of the invention are capable of binding a compound having any one of formulae 3, 4a-15a, 4b-15b, 3c, 4c, 7c or 10c. As used herein, the term binding entities includes antibodies and other polypeptides capable of binding cholesterol ozonation products.

In one embodiment, the antibody or binding entity can selectively bind a compound having any one of formulae 3, 4a-15a, 4b-15b, 3c, 4c, 7c or 10c. In another embodiment the antibody or binding entity can bind more than one compound having of formulae 3, 4a-15a, 4b-15b, 3c, 4c, 7c or 10c. Specific examples of antibody preparations were raised against compounds having formula 13a, 14a, 13b, 14b or 15a. In particular, hybridomas KA1-11C5 and KA1-7A6 provide antibody preparations that were raised against a compound having formula 15a. Hybridomas KA2-8F6 and KA2-1E9 provide antibody preparations that were raised against a compound having formula 14a.

Hybridomas KA1-11C5 and KA1-7A6, raised against a compound having formula 15a, were deposited under the terms of the Budapest Treaty on Aug. 29, 2003 with the American Type Culture Collection (10801 University Blvd., Manassas, Va., 20110-2209 USA (ATCC)) as ATCC Accession No. ATCC Numbers PTA-5427 and PTA-5428. Hybridomas KA2-8F6 and KA2-1E9, raised against a compound having formula 14a, were deposited with the ATCC under the terms of the Budapest Treaty also on Aug. 29, 2003 as ATCC Accession No. ATCC PTA-5429 and PTA-5430.

The invention also provides antibodies made by available procedures that can bind an ozonation product of cholesterol. The binding domains of such antibodies, for example, the CDR regions of these antibodies, can be transferred into or utilized with any convenient binding entity backbone.

Antibody molecules belong to a family of plasma proteins called immunoglobulins, whose basic building block, the immunoglobulin fold or domain, is used in various forms in many molecules of the immune system and other biological recognition systems. A standard antibody is a tetrameric structure consisting of two identical immunoglobulin heavy chains and two identical light chains and has a molecular weight of about 150,000 daltons.

The heavy and light chains of an antibody consist of different domains. Each light chain has one variable domain (VL) and one constant domain (CL), while each heavy chain has one variable domain (VH) and three or four constant domains (CH). See, e.g., Alzari, P. N., Lascombe, M.-B. & Poljak, R. J. (1988) Three-dimensional structure of antibodies. Annu. Rev. Immunol. 6, 555-580. Each domain, consisting of about 110 amino acid residues, is folded into a characteristic β-sandwich structure formed from two β-sheets packed against each other, the immunoglobulin fold. The VH and VL domains each have three complementarity determining regions (CDR1-3) that are loops, or turns, connecting β-strands at one end of the domains. The variable regions of both the light and heavy chains generally contribute to antigen specificity, although the contribution of the individual chains to specificity is not always equal. Antibody molecules have evolved to bind to a large number of molecules by using six randomized loops (CDRs).

Immunoglobulins can be assigned to different classes depending on the amino acid sequences of the constant domain of their heavy chains. There are at least five (5) major classes of immunoglobulins: IgA, IgD, IgE, IgG and IgM.

Several of these may be further divided into subclasses (isotypes), for example, IgG-1, IgG-2, IgG-3 and IgG-4; IgA-1 and IgA-2. The heavy chain constant domains that correspond to the IgA, IgD, IgE, IgG and IgM classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of variable domains differ extensively in sequence from one antibody to the next. The variable domains are for binding and determine the specificity of each particular antibody for its particular antigen. However, the variability is not evenly distributed through the variable domains of antibodies.

Instead, the variability is concentrated in three segments called complementarity determining regions (CDRs), also known as hypervariable regions in both the light chain and the heavy chain variable domains.

The more highly conserved portions of variable domains are called framework (FR) regions. The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from another chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific cholesterol ozonation product or derivative thereof.

Moreover, the binding regions, or CDR, of antibodies can be placed within the backbone of any convenient binding entity polypeptide. In preferred embodiments, in the context of methods described herein, an antibody, binding entity or fragment thereof is used that is immunospecific for any of compounds of formulae 3-15, as well as the haptens and derivatives thereof, including the hydrazone derivatives.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called Fab fragments, each with a single antigen binding site, and a residual Fc fragment. Fab fragments thus have an intact light chain and a portion of one heavy chain. Pepsin treatment yields an F(ab′)₂ fragment that has two antigen binding fragments that are capable of cross-linking antigen, and a residual fragment that is termed a pFc′ fragment. Fab′ fragments are obtained after reduction of a pepsin digested antibody, and consist of an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site. As used herein, “functional fragment” with respect to antibodies, refers to Fv, F(ab) and F(ab′)₂ fragments.

Additional fragments can include diabodies, linear antibodies, single-chain antibody molecules, and multispecific antibodies formed from antibody fragments. Single chain antibodies are genetically engineered molecules containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, where the fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad Sci. USA 90: 6444-6448 (1993).

Antibody fragments contemplated by the invention are therefore not full-length antibodies. However, such antibody fragments can have similar or improved immunological properties relative to a full-length antibody. Such antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more.

In general, an antibody fragment of the invention can have any upper size limit so long as it is has similar or improved immunological properties relative to an antibody that binds with specificity to an ozonation product of cholesterol. For example, smaller binding entities and light chain antibody fragments can have less than about 200 amino acids, less than about 175 amino acids, less than about 150 amino acids, or less than about 120 amino acids if the antibody fragment is related to a light chain antibody subunit. Moreover, larger binding entities and heavy chain antibody fragments can have less than about 425 amino acids, less than about 400 amino acids, less than about 375 amino acids, less than about 350 amino acids, less than about 325 amino acids or less than about 300 amino acids if the antibody fragment is related to a heavy chain antibody subunit.

Antibodies directed against the cholesterol ozonation products of the invention can be made by any available procedure. Methods for the preparation of polyclonal antibodies are available to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

Monoclonal antibodies can also be employed in the invention. The term “monoclonal antibody” as used herein refers to an antibody obtained from a population of substantially homogeneous antibodies. In other words, the individual antibodies comprising the population are identical except for occasional naturally occurring mutations in some antibodies that may be present in minor amounts. Monoclonal antibodies are highly specific, being directed against a single antigenic site. Furthermore, in contrast to polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass. Fragments of such antibodies can also be used, so long as they exhibit the desired biological activity. See U.S. Pat. No. 4,816,567; Morrison et al. Proc. Natl. Acad Sci. 81, 6851-55 (1984).

The preparation of monoclonal antibodies likewise is conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of antibodies are available to those skilled in the art. For example, the monoclonal antibodies to be used in accordance with the present invention may be made by the hybridoma method as described above or may be made by recombinant methods, e.g., as described in U.S. Pat. No. 4,816,567. Monoclonal antibodies for use with the present invention may also be isolated from phage antibody libraries using the techniques described in Clackson et al. Nature 352: 624-628 (1991), as well as in Marks et al., J. Mol Biol. 222: 581-597 (1991).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression of nucleic acids encoding the antibody fragment in a suitable host. Antibody fragments can be obtained by pepsin or papain digestion of whole antibodies conventional methods. For example, antibody fragments can be produced by enzymatic cleavage of antibodies with pepsin to provide a 5S fragment described as F(ab′)₂. This fragment can be further cleaved using a thiol reducing agent, and optionally using a blocking group for the sulfhydryl groups resulting from cleavage of disulfide linkages, to produce 3.5S Fab′ monovalent fragments. Alternatively, enzymatic cleavage using pepsin produces two monovalent Fab′ fragments and an Fc fragment directly. These methods are described, for example, in U.S. Pat. Nos. 4,036,945 and No. 4,331,647, and references contained therein. These patents are hereby incorporated by reference in their entireties.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Entomology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Entomology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998).

While standardized procedures are available to generate antibodies, the size of antibodies, the multi-stranded structure of antibodies and the complexity of six binding loops present in antibodies constitute a hurdle to the improvement and the manufacture of large quantities of antibodies. Hence, the invention further contemplates using binding entities, which comprise polypeptides that can recognize and bind to an ozonation product of cholesterol.

A number of proteins can serve as protein scaffolds to which binding domains for cholesterol ozonation products can be attached and thereby form a suitable binding entity. The binding domains bind or interact with the cholesterol ozonation products of the invention while the protein scaffold merely holds and stabilizes the binding domains so that they can bind. A number of protein scaffolds can be used. For example, phage capsid proteins can be used. See Review in Clackson & Wells, Trends Biotechnol. 12:173-184 (1994). Phage capsid proteins have been used as scaffolds for displaying random peptide sequences, including bovine pancreatic trypsin inhibitor (Roberts et al., PNAS 89:2429-2433 (1992)), human growth hormone (Lowman et al., Biochemistry 30:10832-10838 (1991)), Venturini et al., Protein Peptide Letters 1:70-75 (1994)), and the IgG binding domain of Streptococcus (O'Neil et al., Techniques in Protein Chemistry V (Crabb, L, ed.) pp. 517-524, Academic Press, San Diego (1994)). These scaffolds have displayed a single randomized loop or region that can be modified to include binding domains for cholesterol ozonation products.

Researchers have also used the small 74 amino acid α-amylase inhibitor Tendamistat as a presentation scaffold on the filamentous phage M13. McConnell, S. J., & Hoess, R. H., J. Mol. Biol. 250:460-470 (1995). Tendamistat is a β-sheet protein from Streptomyces tendae. It has a number of features that make it an attractive scaffold for binding peptides, including its small size, stability, and the availability of high resolution NMR and X-ray structural data. The overall topology of Tendamistat is similar to that of an immunoglobulin domain, with two β-sheets connected by a series of loops. In contrast to immunoglobulin domains, the β-sheets of Tendamistat are held together with two rather than one disulfide bond, accounting for the considerable stability of the protein. The loops of Tendamistat can serve a similar function to the CDR loops found in immunoglobulins and can be easily randomized by in vitro mutagenesis. Tendamistat is derived from Streptomyces tendae and may be antigenic in humans. Hence, binding entities that employ Tendamistat are preferably employed in vitro.

Fibronectin type III domain has also been used as a protein scaffold to which binding entities can be attached. Fibronectin type III is part of a large subfamily (Fn3 family or s-type Ig family) of the immunoglobulin superfamily. Sequences, vectors and cloning procedures for using such a fibronectin type III domain as a protein scaffold for binding entities (e.g. CDR peptides) are provided, for example, in U.S. Patent Application Publication 20020019517. See also, Bork, P. & Doolittle, R. F. (1992) Proposed acquisition of an animal protein domain by bacteria. Proc. Natl. Acad. Sci. USA 89, 8990-8994; Jones, E. Y. (1993) The immunoglobulin superfamily Curr. Opinion Struct. Biol. 3, 846-852; Bork, P., Hom, L. & Sander, C. (1994) The immunoglobulin fold. Structural classification, sequence patterns and common core. J. Mol. Biol. 242, 309-320; Campbell, I. D. & Spitzfaden, C. (1994) Building proteins with fibronectin type III modules Structure 2, 233-337; Harpez, Y. & Chothia, C. (1994).

In the immune system, specific antibodies are selected and amplified from a large library (affinity maturation). The combinatorial techniques employed in immune cells can be mimicked by mutagenesis and generation of combinatorial libraries of binding entities. Variant binding entities, antibody fragments and antibodies therefore can also be generated through display-type technologies. Such display-type technologies include, for example, phage display, retroviral display, ribosomal display, and other techniques. Techniques available in the art can be used for generating libraries of binding entities, for screening those libraries and the selected binding entities can be subjected to additional maturation, such as affinity maturation. Wright and Harris, supra., Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743.

The invention therefore also provides methods of mutating antibodies, CDRs or binding domains to optimize their affinity, selectivity, binding strength and/or other desirable properties. A mutant binding domain refers to an amino acid sequence variant of a selected binding domain (e.g. a CDR). In general, one or more of the amino acid residues in the mutant binding domain is different from what is present in the reference binding domain. Such mutant antibodies necessarily have less than 100% sequence identity or similarity with the reference amino acid sequence. In general, mutant binding domains have at least 75% amino acid sequence identity or similarity with the amino acid sequence of the reference binding domain. Preferably, mutant binding domains have at least 80%, more preferably at least 85%, even more preferably at least 90%, and most preferably at least 95% amino acid sequence identity or similarity with the amino acid sequence of the reference binding domain.

For example, affinity maturation using phage display can be utilized as one method for generating mutant binding domains. Affinity maturation using phage display refers to a process described in Lowman et al., Biochemistry 30(45): 10832-10838 (1991), see also Hawkins et al., J. Mol Biol. 254: 889-896 (1992). While not strictly limited to the following description, this process can be described briefly as involving mutation of several binding domains or antibody hypervariable regions at a number of different sites with the goal of generating all possible amino acid substitutions at each site. The binding domain mutants thus generated are displayed in a monovalent fashion from filamentous phage particles as fusion proteins. Fusions are generally made to the gene III product of M13. The phage expressing the various mutants can be cycled through several rounds of selection for the trait of interest, e.g. binding affinity or selectivity. The mutants of interest are isolated and sequenced. Such methods are described in more detail in U.S. Pat. No. 5,750,373, U.S. Pat. No. 6,290,957 and Cunningham, B. C. et al., EMBO J. 13(11), 2508-2515 (1994).

Therefore, in one embodiment, the invention provides methods of manipulating binding entity or antibody polypeptides or the nucleic acids encoding them to generate binding entities, antibodies and antibody fragments with improved binding properties that recognize the cholesterol ozonation products.

Such methods of mutating portions of an existing binding entity or antibody involve fusing a nucleic acid encoding a polypeptide that encodes a binding domain for a cholesterol ozonation product to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage, and selecting phage that bind to an ozonation product of cholesterol.

Accordingly, the invention provides antibodies, antibody fragments, and binding entity polypeptides that can recognize and bind to a cholesterol ozonation product, hapten or cholesterol derivative. The invention further provides methods of manipulating those antibodies, antibody fragments, and binding entity polypeptides to optimize their binding properties or other desirable properties (e.g., stability, size, ease of use).

Such antibodies, antibody fragments, and binding entity polypeptides can be modified to include a label or reporter molecule useful for detecting the presence of the antibody. As used herein, a label or reporter molecule is any molecule that can be associated with an antibody, directly or indirectly, and that results in a measurable, detectable signal, either directly or indirectly. Many such labels can be incorporated into or coupled onto an antibody or binding entity are available to those of skill in the art. Examples of labels suitable for use with the antibodies and binding entities of the invention include radioactive isotopes, fluorescent molecules, phosphorescent molecules, enzymes, secondary antibodies, and ligands.

Examples of suitable fluorescent labels include fluorescein (FITC), 5,6-carboxyrnethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. In some embodiments, the fluorescent label is fluorescein (5-carboxyfluorescein-N-hydroxysuccinimide ester) or rhodamine (5,6-tetramethyl rhodamine). Fluorescent labels for combinatorial multicolor used in some embodiments include FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Such fluorescent labels can be obtained from a variety of commercial sources, including Molecular Probes, Eugene. Oreg. and Research Organics, Cleveland, Ohio.

Detection labels that are incorporated into an antibody or binding entity, such as biotin, can be subsequently detected using sensitive methods available in the art. For example, biotin can be detected using streptavidin-alkaline phosphatase conjugate (Tropix., Inc.) that binds to the biotin and subsequently can be detected by chemiluminescence of suitable substrates (for example, the chemiluminescent substrate CSPD: disodium, 3-(4-methoxyspiro-[1,2,-dioxetane-3-2′-(5′-chloro)tricyclo[3.3.1.1.sup.3,7]decane]-4-yl)phenyl phosphate; Tropix, Inc.).

Molecules that combine two or more of these reporter molecules or detection labels can also be used in the invention. Any of the known detection labels can be used with the disclosed antibodies, antibody fragments, binding entities, and methods. Methods for detecting and measuring signals generated by detection labels are also available to those of skill in the art. For example, radioactive isotopes can be detected by scintillation counting or direct visualization; fluorescent molecules can be detected with fluorescent spectrophotometers; phosphorescent molecules can be detected with a scanner or spectrophotometer, or directly visualized with a camera; enzymes can be detected by visualization of the product of a reaction catalyzed by the enzyme. Such methods can be used directly in the disclosed method of detecting ozonation products of cholesterol.

Assays for Cholesterol Ozonation Products

Any assay available to one of skill in the art can be used for detecting cholesterol ozonation products, including assays for detecting cholesterol haptens or cholesterol derivatives that are indicative of cholesterol ozonation. For example, the assay can employ, mass spectroscopy, gas or liquid chromatography, nuclear magnetic resonance, infrared spectroscopy, ultraviolet spectroscopy, visible light spectroscopy or high pressure liquid chromatography. In some embodiments, an immunoassay can be used for detecting any of compounds 3, 4a-15a, 3c, 4c, 7e, 10c or 4b-15b.

Assays can be used to detect ozonation products of cholesterol in test samples obtained from a variety of sources including, for example, serum, plasma, blood, lymph, tissues (e.g. plaque samples), saliva, urine, stool, and other biological samples from a mammal. In some embodiments, the test sample is a tissue sample. However, in other embodiments the test sample is a bodily fluid such as urine, blood or serum. Evaluation of such samples from mammalian subjects permits non-invasive diagnosis of vascular diseases. For example, mammalian fluids can be taken from a subject and assayed for cholesterol ozonation products, either as released factors or as membrane bound factors on cells in the sample fluid.

In some embodiments, an immunoassay is employed. Such an immunoassay can involve any assay method available to one of skill in the art. Examples of immunoassays include radioimmunoassays, competitive binding assays, sandwich assays, and immunoprecipitation assays. Binding entities of the invention can be combined or attached to a detectable label as described herein. The choice of label used will vary depending upon the application and can be made by one skilled in the art.

In the practice of this invention the detectable label may be an enzyme such as horseradish peroxidase or alkaline phosphatase, a paramagnetic ion, a chelate of a paramagnetic ion, biotin, a fluorophore, a chromophore, a heavy metal, a chelate of a heavy metal, a compound or element which is opaque to X-rays, a radioisotope, or a chelate of a radioisotope.

Radioisotopes useful as detectable labels include such isotopes as iodine-123, iodine-125, iodine-128, iodine-131, or a chelated metal ion of chromium-51, cobalt-57, gallium-67, indium-111, indium-113m, mercury-197, selenium-75, thallium-201, technetium-99m, lead-203, strontium-85, strontium-87, gallium-68, samarium-153, europium-157, ytterbium-169, zinc-62, or rhenium-188.

Paramagnetic ions useful as detectable label s include such ions as chromium (III), manganese (II), iron (III), iron (II), cobalt (II), nickel (II), copper (II), praseodymium (III), neodymium (III), samarium (III), gadolinium (III), terbium (III), dysprosium (III), holmium (III), erbium (III), or ytterbium (III).

Radioimmunoassays typically use radioactivity in the measurement of complexes between binding entities (e.g. antibodies) and cholesterol ozonation products. In such a method, the binding entity is radio-labeled. The binding entity is reacted with unlabeled cholesterol ozonation product. The radio-labeled complex is then separated from unbound material, for example, by precipitation followed by centrifugation. Once the complex between the radio-labeled binding entity and the cholesterol ozonation product is separated from the unbound material, the amount of complex is quantified either by measuring the radiation directly or by observing the effect that the radiolabel has on a fluorescent molecule, such as dephenyloxazole (DPO). The latter approach requires less radioactivity and is more sensitive. This approach, termed scintillation, measures the fluorescent transmission of a dye solution that has been excited by a radiolabel, such as ³H or ³²P. The extent of binding is determined by measuring the intensity of the fluorescence released from the fluorescent particles. This method, termed scintillation proximity assay (SPA), has the advantage of being able to measure binding entity complexes formed in situ without the need for washing off unbound radioactive binding entity.

Competitive binding assays rely on the ability of a labeled standard to compete with the test sample analyte for binding with a limited amount of binding entity. The labeled standard may be an ozonation product of cholesterol or an immunologically reactive hapten or derivative thereof. The amount of test sample is inversely proportional to the amount of standard that becomes bound to the binding entities. To facilitate determining the amount of standard that becomes bound, the binding entities employed are generally made insoluble either before or after the competition. This is done so that the standard and analyte that are bound to the binding entities may be conveniently separated from the standard and analyte that remain unbound.

Sandwich assays involve the use of two binding entities, each capable of binding to a different immunogenic portion, or epitope, of the product to be detected. In a sandwich assay, the test sample analyte is bound by a first binding entity which is immobilized on a solid support, and thereafter a second binding entity binds to the analyte, thus forming an insoluble three part complex (David & Greene, U.S. Pat. No. 4,376,110). The second binding entity may itself by labeled with a detectable moiety (direct sandwich assays) or may be measured using a third binding entity that binds the second bonding entity and is labeled with a detectable moiety (indirect sandwich assay). For example, one type of sandwich assay is an ELISA assay, in which case the detectable moiety is an enzyme.

Typically, sandwich assays include “forward” assays in which the binding entity bound to the solid phase is first contacted with the sample being tested to extract the cholesterol ozonation product from the sample by formation of a binary solid phase complex between the immobilized binding entity and the cholesterol ozonation product. After a suitable incubation period, the solid support is washed to remove unbound fluid sample, including unreacted cholesterol ozonation product, if any. The solid support is then contacted with the solution containing an unknown quantity of labeled binding entity (which functions as a label or reporter molecule). After a second incubation period to permit the labeled binding entity to react with the complex between the immobilized binding entity and the cholesterol ozonation product, the solid support is washed a second time to remove the unreacted labeled binding entity. This type of forward sandwich assay may be a simple “yes/no” assay to determine whether a cholesterol ozonation product is present in the test sample.

Other types of sandwich assays that may be used include the so-called “simultaneous” and “reverse” assays. A simultaneous assay involves a single incubation step wherein the labeled and unlabeled binding entities are, at the same time, both exposed to the sample being tested. The unlabeled binding entity is immobilized onto a solid support, while the labeled binding entity is free in solution with the test sample. After the incubation is completed, the solid support is washed to remove unreacted sample and uncomplexed labeled binding entity. The presence of labeled binding entity associated with the solid support is then determined as it would be in a conventional “forward” sandwich assay.

In a “reverse” assay, stepwise addition is utilized, first of a solution of labeled binding entity to a test sample, followed by incubation, and then later by addition of an unlabeled binding entity bound to a solid support. After a second incubation, the solid phase is washed in conventional fashion to free it of the residue of the sample being tested and the solution of unreacted labeled binding entity. The determination of labeled binding entity associated with a solid support is then determined as in the “simultaneous” and “forward” assays.

In addition to their diagnostic utility, the binding entities of the present invention are useful for monitoring the progression of vascular disease in a subject by examining the levels of cholesterol ozonation products in tissues, cells or serum samples over time. Changes in the levels of cholesterol ozonation products over time may indicate further progression of the vascular or heart disease in the subject.

Vascular Diseases

The vascular diseases diagnosed by the present invention are vascular diseases of mammals. The word mammal means any mammal. Some examples of mammals include, for example, pet animals, such as dogs and cats; farm animals, such as pigs, cattle, sheep, and goats; laboratory animals, such as mice and rats; primates, such as monkeys, apes, and chimpanzees; and humans. In some embodiments, humans are preferably diagnosed by the methods of the invention.

The invention relates to methods for detecting or diagnosing a vascular condition, or a circulatory condition involving deposit of cholesterol, and ozonation of cholesterol. Such a condition can be associated with loss, injury or disruption of the vasculature within an anatomical site or system. The term “vascular condition” or “vascular disease” refers to a state of vascular tissue where blood flow is, or can become, impaired.

Many pathological conditions can lead to vascular diseases that are associated deposition of cholesterol. Examples of vascular conditions that can be detected or diagnosed with the compositions and methods of the invention include atherosclerosis (or arteriosclerosis), preeclampsia, peripheral vascular disease, heart disease, and stroke. Thus, the invention is directed to methods of treating diseases such as stroke, atherosclerosis, acute coronary syndromes including unstable angina, thrombosis and myocardial infarction, plaque rupture, both primary and secondary (in-stent) restenosis in coronary or peripheral arteries, transplantation-induced sclerosis, peripheral limb disease, intermittent claudication and diabetic complications (including ischemic heart disease, peripheral artery disease, congestive heart failure, retinopathy, neuropathy and nephropathy), or thrombosis.

Kits

Kits for detecting cholesterol ozonation products in a test sample are also included in the invention. In one embodiment, the kit comprises a container containing a binding entity or antibody that specifically binds to an ozonation product of cholesterol. The binding entity or antibody can have a directly attached or indirectly associated detection label or reporter molecule. The binding entity or antibody can also be provided in liquid form or it can be attached to a solid phase, for example, as is needed for use in any convenient immunoassay procedure.

The kits of the invention can also contain another container comprising an ozonation product of cholesterol that can be used, for example, as a control or standard in an assay for an ozonation product of cholesterol.

The kits of the invention can further contain another container comprising a reagent that can react with cholesterol to generate a product that can readily be detected by any of the binding entities or antibodies of the invention.

The kits of the invention can also contain a third container comprising a detection label or reporter molecule for detecting the binding entity, antibody or a complex between the binding entity/antibody and an ozonation product of cholesterol.

These kits can also comprise containers with tools useful for collecting test samples (such as blood, plasma, serum, urine, saliva, and stool). Such tools include lancets, tubes and absorbent paper or cloth for collecting and stabilizing blood; swabs for collecting and stabilizing saliva; cups for collecting and stabilizing urine or stool samples. Collection materials, such as tubes, papers, cloths, swabs, cups and the like, may optionally be treated to avoid denaturation or irreversible adsorption of the sample. These collection materials also may be treated with, or contain, preservatives, stabilizers or antimicrobial agents to help maintain the integrity of the specimens.

The invention is further illustrated by the following non-limiting Examples.

### EXAMPLE 1

### Materials and Methods

This Example provides materials and methods for some of the experiments described herein.

Operative isolation and handling of atherosclerotic artery specimens. Tissue samples were obtained by carotid endarterectomy. The samples contained atherosclerotic plaque and some adherent intima and media. The protocol for plaque analysis was approved by the Scripps Clinic Human Subjects Committee and patient consent was obtained prior to surgery. Fresh carotid endarterectomy tissue was analyzed within 30 min of operative removal. Note that the plaque samples were neither stored nor preserved. All analytical manipulations were complete within 2 h of surgical removal. No fixatives were added to the specimens.

Oxidation of indigo carmine 1 by human atherosclerotic artery specimens. Endarterectomy specimens (n=15), isolated as described above, were divided into two sections of approximately equal wet weight (±5%). Each specimen was placed into phosphate buffered saline (PBS, pH 7.4, 1.8 mL) containing indigo carmine 1 (200 μM, Aldrich) and bovine catalase (100 μg). Indigo carmine 1 was added to act as a chemical trap for ozone. Takeuchi et al., Anal. Chim. Acta 230, 183 (1990); Takeuchi et al., Anal. Chem. 61, 619 (1989). Phorbal myristate (PMA, 40 μg in 0.2 mL of DMSO) or DMSO (0.2 mL) was added as an activator of protein kinase C. Each sample was homogenized using a tissue homogenizer for 10 min and then centrifuged (10,000 rpm for 10 min). The supernatants were decanted, passed through a filter (0.2 μm) and the filtrate was analyzed for the presence of isatin sulfonic acid 2 using quantitative HPLC.

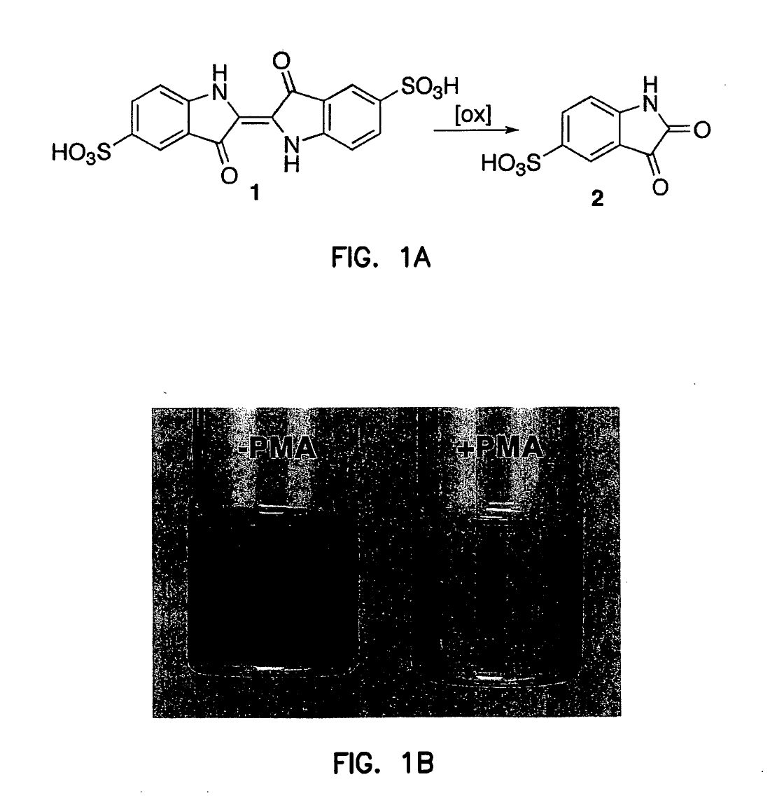

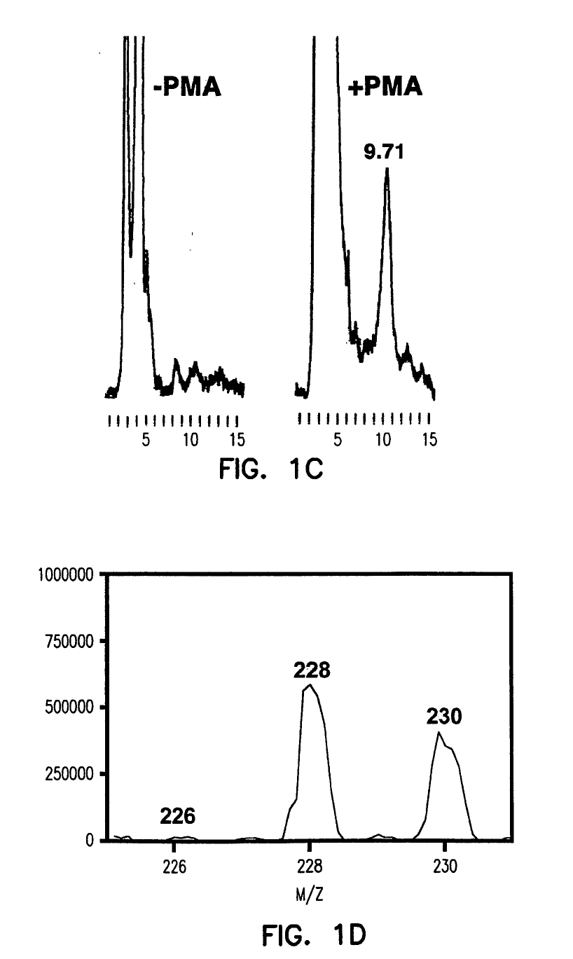

As shown by FIG. 1B, the visible absorbance of indigo carmine 1 was bleached and the reaction gave rise to a new chemical species that was detected using quantitative HPLC (Table 1), and that was identified as isatin sulfonic acid 2 (see also FIG. 1A).

HPLC assay for quantification of isatin sulfonic acid 2. HPLC analysis was performed on a Hitachi D-7000 machine, with a L-7200 autosampler, a L-7100 pump and a L-7400 u.v. detector (254 nm). The L-7100 was controlled using Hitachi-HSM software on a Dell GX150 PC computer. LC conditions were a Spherisorb RP-C₁₈ column and acetonitrile:water (0.1% TFA) (80:20) mobile phase at 1,2 mL/min. Isatin sulfonic acid 2 had a retention time, R_{T}, of about 9.4 min. Quantification was performed by comparison of peak areas to standard curves of peak area vs. concentration of authentic samples using GraphPad v3.0 software for Macintosh (Table 1).

TABLE 1
Isatin sulfonic acid 2 (ISA) formed by activated
atherosclerotic artery material.

| Sample | ISA nmol/mg |
|---|---|
| 1 | 27.3 |
| 2 | 54.4 |
| 3 | 27.6 |
| 4 | 1.0 |
| 5 | 30.1 |
| 6 | 238.3 |
| 7 | 39.4 |
| 8 | 152.9 |
| 9 | 127 |
| 10 | 262.1 |
| 11 | 27.9 |
| 12 | 64.6 |
| 13 | 1.4 |
| 14 | 3.2 |
| 15 | 32.1 |

Mean ± SEM = 72.62 ± 21.69

Oxidation of indigo carmine 1 by human atherosclerotic artery specimens in H₂¹⁸O. This experiment conducted as described in the indigo carmine assay above with the following exceptions. First, each plaque specimen (n=2) was added to phosphate buffer (10 mM, pH 7.4) in greater than 95% H₂¹⁸O. Second, the filtrate was desalted on a PD10 column and analyzed by negative electrospray mass spectrometry on a Finnegan electrospray mass spectrometer. The raw ion abundance data was extracted into Graphpad Prism v 3.0 fornat for presentation.

These experiments indicate that in the presence of plaque material and H₂ ¹⁸O (>95% ¹⁸O), the ¹⁸O isotope is incorporated into the lactam carbonyl of isatin sulfonic acid 2. Because only ozone could oxidatively cleave the double bond of indigo carmine 1 and promote isotope incorporation into the lactam carbonyl of isatin sulfonic acid 2 from H₂¹⁸O, ozone was likely the reactive oxygen species that oxidized indigo carmine 1. Hence, ozone is generated within atherosclerotic lesions. See also, P. Wentworth Jr. et al., Science 298, 2195 (2002); B. M. Babior, C. Takeuchi, J. Ruedi, A. Guitierrez, P. Wentworth Jr., Proc. Natl. Acad. Sci. U.S.A. 100, 3920 (2003); P. Wentworth Jr. et al., Proc. Natl. Acad. Sci. U.S.A. 100, 1490 (2003).

Extraction and derivatization procedure of aldehydes from atheromatous artery specimens. Endarterectomy specimens isolated as described above were divided into two sections of approximately equal wet weight (±5%). Each specimen was placed into phosphate buffered saline (PBS, pH 7.4, 1.8 mL) containing bovine catalase (100 μg) and either phorbol myristate (40 μg in 0.2 mL of DMSO) or DMSO (0.2 mL). Each sample was homogenized using a tissue homogenizer for 10 min. The homogenized endarterectomy samples, isolated as described above, were then washed with dichloromethane (DCM, 3×5 mL). The combined organic fractions were evaporated in vacuo. The residue was dissolved in ethanol (0.9 mL) and a solution of 2,4-dinitrophenyl hydrazine (100 μL, 2 mM, and 1N HCl) in ethanol was added. Nitrogen was bubbled through the solution for 5 min and then the solution was stirred for 2 h. The resultant suspension was filtered through a 0.22 μm filter and the filtrate was analyzed by the HPLC assay vide infra. When cholesterol 3 (1-20 μM) was treated under these conditions, no 4a or 5a was formed. The amount of 4b detected in atheromatous artery extracts both prior to and after PMA addition was subjected to a student two tail t-test analysis to determine the significance of PMA-addition on 4a levels in the artery extracts (p<0.05 was considered to be significant) and was determined with Graphpad v3.0 software for Macintosh.

During the derivatization of 4a under these conditions, about 20% of 4a was converted into 5b over a range of 4a concentrations (5 to 100 μM). These data indicate that a measured amount of 5a, exceeding 20% of the 4a present in the same plaque samples, arose from ozonolysis of 3 followed by aldolization. The extent of conversion of 4a into 6b under the employed derivatization conditions was consistently <2% over a range of 4a concentrations (5 to 100 μM). These observations indicate that the amount of 6a present within the plaque extracts that exceeds 2% of the amount of ketoaldehyde 4a, was present prior to derivatization and has arisen from the ozonolysis product 4a by β-elimination of water.

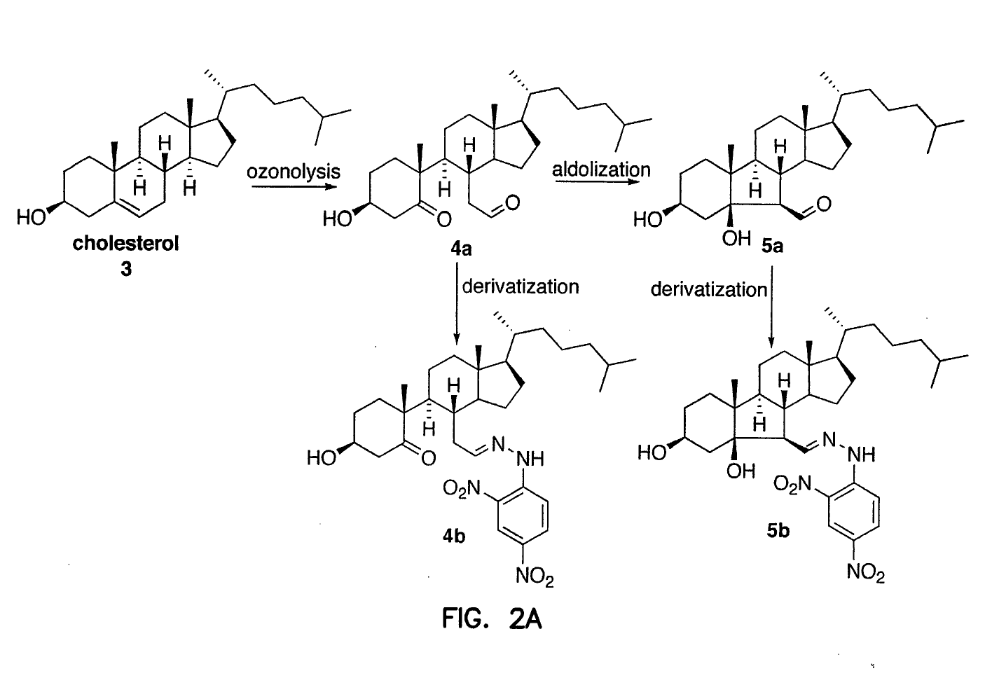

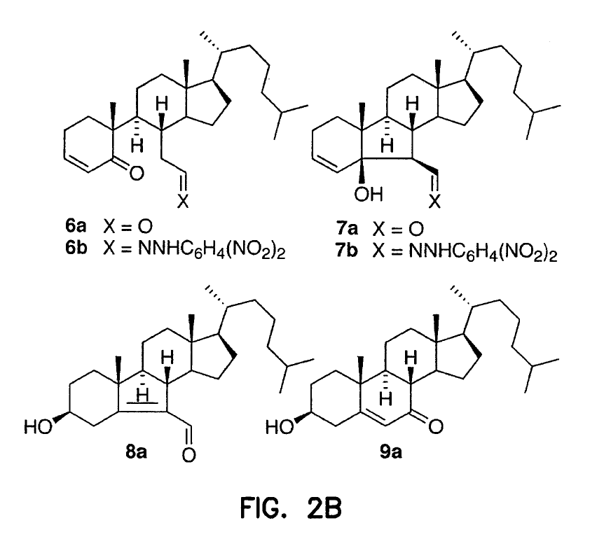

In addition to the three major hydrazone products 4b-6b, the hydrazone derivative of 7a (called 7b) was detected in trace amounts (<5 pmol/mg) in several plaque extracts (R_{T}˜26 min, [M−H]⁻ 579, SOM FIGS. 2 & 4). Compound 7a is the A-ring dehydration product of 5a. The amount of 7b in the derivatized plaque extracts was approaching the detection limit of the HPLC assay employed so a complete analytical investigation of this compound in all the plaque samples was not performed. The configurational assignments of compounds 7a and 7b were based on a ¹H—¹H ROESY experiment of the synthetic material 7b.

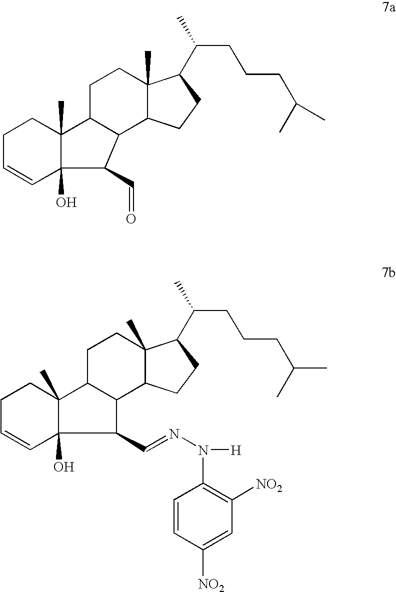

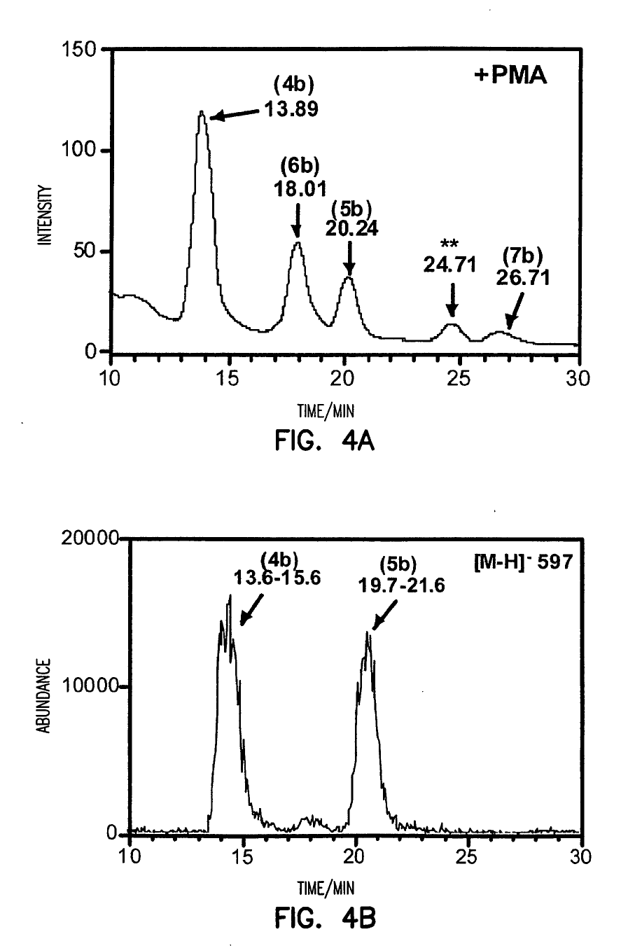

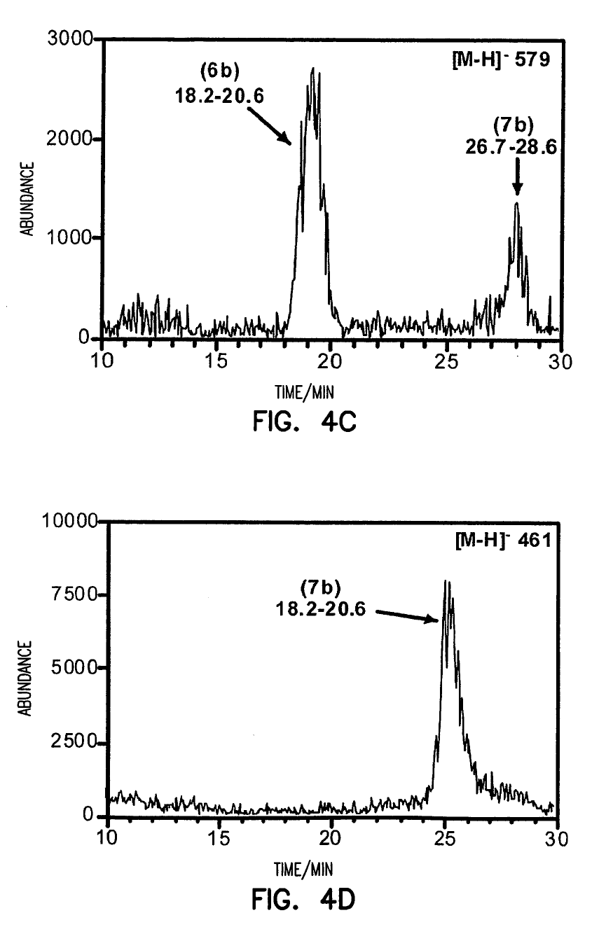

Synthesized preparations of compounds 6b, 7a, 7b, 8a and 9a were employed for identification of the compound having R_{T}˜26 min peak [M−H]⁻ 579 in FIG. 4.

HPLC-MS analysis of hydrazones. HPLC-MS analysis was performed on a Hitachi D-7000 machine, with a L-7200 autosampler (regular injection volume 10 μl), a L-7100 pump and either a L-7400 u.v. detector (360 nm) or a L-7455 diode array detector (200-400 nm) and an in-line M-8000 ion trap mass-spectrometer (in negative ion mode). The L-7100 and M-8000 were controlled using Hitachi-HSM software on a Dell GX150 PC computer. HPLC was performed using a Vydec C₁₈ reversed phase column. An isocratic mobile phase was employed (75% acetonitrile, 20% methanol and 5% water) at 0.5 mL/min. Peak height and area was determined using Hitachi D7000 chromatography station software and converted to concentrations by comparison to standard curves of authentic materials. Under these conditions the detection limit for hydrazones 4b-6b was between 1-10 nM. No resolution of the cis and trans hydrazone isomers was obtained using this HPLC system.

A representative HPLC-MS of extracted and derivatized atherosclerotic material is shown in FIG. 4. The retention times and mass ratios of several authentic samples of key hydrazone compounds are shown in Table 2.

TABLE 2
LCMS analysis of authentic hydrazones.

| hydrazone | R_{T}/min | [M − H]⁻ |
|---|---|---|
| 4b | 13.9 | 597 |
| 5b | 20.3 | 597 |
| 6b | 18.0 | 579 |
| 7b | 26.8 | 579 |
| ^{a,d}8b | 26.6 | 579 |
| ^{b}9b | 16.5 | 579 |
| ^{c}10b | 48.2 | 561 |

^{a}The hydrazone of authentic aldehyde 8a was prepared by the derivatization procedure above, the aldehyde 8a was not independently synthesized and purified.
^{b}The hydrazone of commercially-available ketone 9a was prepared by the derivatization procedure described above, and was not independently synthesized and purified.
^{c}The hydrazone of authentic aldehyde 10a was prepared by the derivatization procedure above, and was not independently synthesized and purified.
^{d}Differentiation between 8b and 9b was made based on their u.v. spectra [measured by a Hitachi L-7455 diode array detector (200-400 nm)]. The α,β-unsaturated hydrazone 8b had a λₘₐₓ of 435 nm, whereas hydrazone 9b had a λₘₐₓ of 416 nm.

Analysis of plasma samples for aldehydes 4a and 5a. Plasma samples were obtained from patients (n=8) who were scheduled to undergo carotid endarterectomy within 24 h. All such plasma samples were analyzed for the presence of 4a and 5a three days after sample collection. Control plasma samples were obtained from random patients (n=15) attending a general medical clinic and were analyzed 7 days after collection. In a typical procedure, plasma in EDTA (1 ml) was washed with dichloromethane (DCM, 3×1 mL). The combined organic fractions were evaporated in vacuo. The residue was dissolved in methanol (0.9 mL) and a solution of 2,4-dinitrophenyl hydrazine (100 μL, 0.01 M, Lancaster) and 1N HCl in ethanol was added. Nitrogen was bubbled through the solution for 5 min and then the solution was stirred for 2 h. The resultant solution was filtered through a 0.22 μm filter and the filtrate was analyzed by the HPLC assay vide supra. Preliminary investigations revealed that the amount of 5a that can be extracted from plasma decreases by about 5% per day.

Preparation of authentic samples 4a, 4b, 5a, 5b, 6a, 6b, 7a, 7b, 8a, and 8b General Methods. Unless otherwise stated, all reactions were performed under an inert atmosphere with dry reagents, solvents, and flame-dried glassware. All starting materials were purchased from Aldrich, Sigma, Fisher, or Lancaster and used as received. Ketone 9a was obtained from Aldrich. All flash column chromatography was performed using silica gel 60 (230-400 mesh). Preparative thin layer chromatography (TLC) was performed using Merck (0.25, 0.5, or 1 mm) coated silica gel Kieselgel 60 F₂₅₄ plates. ¹H NMR spectra were recorded on Bruker AMX-600 (600 MHz), AMX-500 (500 MHz), AMX-400 (400 MHz), or AC-250 (250 MHz) spectrometers. ¹³C NMR spectra were recorded on a Bruker AMX-500 (125.7 MHz) or AMX-400 (100.6 MHz) spectrometer. Chemical shifts are reported in parts per million (ppm) on the δ scale from an internal standard. High-resolution mass spectra were recorded on a VG ZAB-VSE instrument.

3βHydroxy-5-oxo-5,6-secocholestan-6-al (4a). This compound was synthesized as generally described in K. Wang, E. Bermúdez, W. A. Pryor, Steroids 58, 225 (1993). A solution of cholesterol 3 (1 g, 2.6 mmol) in chloroform-methanol (9:1) (100 ml) was ozonized at dry ice temperature for 10 min. The reaction mixture was evaporated and stirred with Zn powder (650 mg, 10 mmol) in water-acetic acid (1:9, 50 ml) for 3 h at room temperature. The reduced mixture was diluted with dichloromethane (100 ml) and washed with water (3×50 ml). The combined organic fractions were dried over sodium sulfate and evaporated to dryness in vacuo. The residue was purified using silica-gel chromatography [ethyl acetate-hexane (25:75)] to give the title compound 4a as a white solid (820 mg, 76%):

¹H NMR (CDCl₃) δ 9.533 (s, 1H, CHO), 4.388 (m, 1H, H-3), 3.000 (dd, J=14.0, 4.0 Hz, 1H, H-4e), 0.927 (s, 3H, CH₃-19), 0.827 (d, J=6.8 Hz, 3H, CH₃-21), 0.782 (d, J=6.8 Hz, 3H, CH₃), 0.778 (d, J=6.8 Hz, 3H, CH₃), 0.603 (s, 3H, CH₃-18); ¹³C NMR (CDCl₃) δ 217.90 (C-5), 202.76 (C-6), 70.81 (C-3), 55.96 (C-17), 54.26 (C-14), 52.52 (C-10), 46.70 (C-4), 44.17 (C-7), 42.43 (C-13), 42.17 (C-9), 39.75 (C-12), 39.33 (C-24), 35.85 (C-22), 35.61 (C-20), 34.58 (C-8), 33.99 (C-1), 27.87 (C-25), 27.73 (C-16), 27.52 (C-2), 25.22 (C-15), 23.62 (C-23), 22.91 (C-11), 22.70 (C-27), 22.44 (C-26), 18.44 (C-21), 17.46 (C-19), 11.42 (C-18). HRMALDITOFMS calcd for C₂₇H₄₆O₃Na (M+Na)⁺ 441.3339, found 441.3355.

2,4-Dinitrophenylhydrazone of 3β-hydroxy-5-oxo-5,6-secocholestan-6-al (4b). This compound was synthesized as generally described in K. Wang, E. Bermúdez, W. A. Pryor, Steroids 58, 225 (1993). 2,4-Dinitrophenylhydrazine (52 mg, 0.26 mmol) and p-toluenesulfonic acid (1 mg, 0.0052 mmol) was added to a solution of ketoaldehyde 4a (100 mg, 0.24 mmol) in acetonitrile (10 ml). The reaction mixture was stirred for 4 h at room temperature, and evaporated to dryness in vacuo. The residue was dissolved in ethyl acetate (10 ml) and washed with water (3×20 ml). The combined organics were dried over sodium sulfate and evaporated to dryness in vacuo. The residue was purified by silica gel chromatography [ethyl acetate-hexane (1:4)] to give the title compound 4b as a yellow solid (100 mg, 70%) and as a mixture of the cis and trans isomers (1:4). Crystallization from hexane-methylene chloride gave trans-4b as yellow needles (30 mg, 21%):

¹H NMR (CDCl₃): δ 10.994 (s, 1H, NH), 9.107 (d, J=2.8 Hz, 1H, H-3′), 8.316 (dd, J=9.6, 2.8 Hz, 1H, H-5′), 7.923 (d, J=9.6 Hz, 1H, H-6′), 7.419 (dd, J=6.0, 3.6 Hz, 1H, H-6), 4.417 (m, 1H, H-3), 2.971 (dd, J=13.6, 4.0 Hz, 1H, H-4e), 1.076 (s, 3H, CH₃-19), 0.915 (d, J=6.4 Hz, 3H, CH₃-21), 0.853 (d, J=6.4 Hz, 3H, CH₃), 0.849 (d, J=6.4 Hz, 3H, CH₃), 0.710 (s, 3H, CH₃-18); ¹³C NMR (CDCl₃) δ 216.05 (C-5), 150.84 (C-6), 144.96 (C-1′), 137.87 (C-4′), 130.23 (C-5′), 128.90 (C-2′), 123.50 (C-3′), 116.52 (C-6′), 71.42 (C-3), 56.07 (C-17), 54.54 (C-14), 52.69 (C-10), 47.34 (C-4), 42.61 (C-13), 42.61 (C-9), 39.82 (C-12), 39.42 (C-24), 36.99 (C-8), 35.96 (C-22), 35.67 (C-20), 34.13 (C-1), 32.65 (C-7), 27.98 (C-16), 27.93 (C-25), 27.90 (C-2), 25.31 (C-15), 23.70 (C-23), 23.12 (C-11), 22.78 (C-27), 22.52 (C-26), 18.56 (C-21), 17.77 (C-19), 11.67 (C-18); HRMALDITOFMS calcd for C₃₃H₅₀N₄O₆Na (M+Na) 621.3622, found 621.3622: λₘₐₓ 360 nm, ε 2.57±0.31×10⁴ M⁻¹cm⁻¹.

3β-Hydroxy-5β-hydroxy-B-norcholestane-6β-carboxaldehyde (5a). This compound was synthesized as generally described in T. Miyamoto, K. Kodama, Y. Aramaki, R. Higuchi, R. W. M. Van Soest, Tetrahedron Letter 42, 6349 (2001). To a solution of ketoaldehyde 4a (800 mg, 1.9 mmol) in acetonitrile-water (20:1, 100 ml) was added of L-proline (220 mg, 1.9 mmol). The reaction mixture was stirred for 2 h at room temperature, evaporated to dryness in vacuo. The residue was dissolved in ethyl acetate (50 ml) and washed with water (3×50 ml). The combined organic fractions were dried over sodium sulfate and evaporated in vacuo. The residue was purified by silica gel chromatography [ethyl acetate-hexane (1:4)] to give the title compound 5a as a white solid (580 mg, 73%):

¹H NMR (CDCl₃) δ 9.689 (d, J=2.8 Hz, 1H, CHO), 4.115 (m, 1H, H-3), 3.565 (s, 1H, 3β-OH), 2.495 (broad s, 1H, 5β-OH), 2.234 (dd, J=9.2, 3.2 Hz, 1H, H-6), 0.920 (s, 3H, CH₃-19), 0.904 (d, J=6.4 Hz, 3H, CH₃-21), 0.854 (d, J=6.8 Hz, 3H, CH₃), 0.850 (d, J=6.8 Hz, 3H, CH₃), 0.705 (s, 3H, CH₃-18); ¹³C NMR (CDCl₃) δ 204.74 (C-7), 84.26 (C-5), 67.33 (C-3), 63.89 (C-9), 56.10 (C-14), 55.67 (C-17), 50.42 (C-6), 45.47 (C-10), 44.72 (C-13), 44.22 (C-4), 40.02 (C-8), 39.67 (C-12), 39.44 (C-24), 36.15 (C-22), 35.58 (C-20), 28.30 (C-16), 27.98 (C-2), 27.91 (C-25), 26.69 (C-1), 24.55 (C-15), 23.78 (C-23), 22.78 (C-27), 22.52 (C-26), 21.54 (C-11), 18.71 (C-21), 18.43 (C-19), 12.48 (C-18). HRMALDITOFMS calcd for C₂₇H₄₆O₃Na (M+Na)⁺ 441.3339, found 441.3351.

2,4-Dinitrophenylhydrazone of 3β-Hydroxy-5β-hydroxy-B-norcholestane-6β-carboxaldehyde (5b). This compound was synthesized as generally described in K. Wang, E. Bermúdez, W. A. Pryor, Steroids 58, 225 (1993). 2,4-Dinitrophenylhydrazine (52 mg, 0.26 mmol) and hydrochloric acid (12 M, 2 drops) was added to a solution of aldehyde 5a (100 mg, 0.24 mmol) in acetonitrile (10 ml). The reaction mixture was stirred for 4 h at room temperature and evaporated to dryness in vacuo. The residue was dissolved in ethyl acetate (10 ml) and was washed with water (3×20 ml). The combined organic fractions were dried over sodium sulfate and evaporated to dryness in vacuo. The residue was purified by silica gel chromatography [ethyl acetate-hexane (1:4)] to give the title compound 5b as a yellow solid (90 mg, 62%) as the trans-5b phenylhydrazone:

¹H NMR (CDCl₃) 11.049 (s, 1H, NH), 9.108 (d, J=2.4 Hz, 1H, H-3′), 8.280 (dd, J=9.6, 2.6 Hz, 1H, H-5′), 7.901 (d, J=9.6 Hz, 1H, H-6′), 7.561 (d, J=7.2 Hz, 1H, H-7), 4.214 (m, 1H, H-3), 3.349 (s, 1H, 3β-OH), 2.337 (dd, J=9.2, 6.8 Hz, 1H, H-6), 0.967 (s, 3H, CH₃-19), 0.917 (d, J=6.8 Hz, 3H, CH₃-21), 0.850 (d, J=6.4 Hz, 3H, CH₃), 0.846 (d, J=6.4 Hz, 3H, CH₃), 0.713 (s, 3H, CH₃-18); ¹³C NMR (CDCl₃) δ 155.18 (C-7), 145.12 (C-1′), 137.51 (C-4′), 129.91 (C-5′), 128.64 (C-2′), 123.57 (C-3′), 116.36 (C-6′), 83.35 (C-5), 67.56 (C-3), 56.34 (C-17), 56.34 (C-9), 55.56 (C-14), 51.47 (C-6), 45.50 (C-10), 44.76 (C-13), 43.62 (C-4), 42.59 (C-8), 39.66 (C-12), 39.43 (C-24), 36.16 (C-22), 35.58 (C-20), 28.50 (C-16), 28.07 (C-2), 27.98 (C-25), 27.70 (C-1), 24.67 (C-15), 23.78 (C-23), 22.78 (C-27), 22.52 (C-26), 21.63 (C-11), 18.75 (C-21), 18.67 (C-19), 12.48 (C-18); HRMALDITOFMS calcd for C₃₃H₅₀N₄O₆Na (M+Na)⁺ 621.3622, found 621.3625. HPLC-MS detection: R_{T} 20.8 min; [M−H]⁻ 597; λₘₐₓ 361 nm, ε 2.47±0.68×10⁴ M⁻¹cm⁻¹.

5-Oxo-5,6-secocholest-3-en-6-al (6a). This compound was synthesized as generally described in P. Yates, S. Stiveer, Can. J. Chem. 66, 1209 (1988). Methanesulfonyl chloride (400 μl, 2.87 mmol) was added dropwise to a stirred solution of ketoaldehyde 4a (300 mg, 0.72 mmol) and triethylamine (65 μl, 0.84 mmol) in CH₂Cl₂ (15 ml) at ice-bath temperature. The resulting solution was stirred for 30 min under argon at 0° C., triethylamine (400 μl, 2.87 mmol) was then added and the solution was warmed to room temperature. After 2 h, the reaction mixture was evaporated to dryness in vacuo. The residue was dissolved in methylene chloride (15 ml) and washed with water (3×20 ml). The combined organic fractions were dried over anhydrous sodium sulfate and evaporated in vacuo. The crude residue was purified by silica gel chromatography [ethyl acetate-hexane (1:9)]. The fractions were evaporated to give aldehyde 6a (153 mg, 53%) as a colorless oil. ¹H NMR (CDCl₃) of shows δ 9.574 (s, 1H, CHO), 6.769 (m, 1H, H-3), 5.822 (d, J=10 Hz, 1H, H-4), 2.512 (dd, J=16.8, 3.6 Hz, 1H, H-7), 1.070 (s, 3H, CH₃-19), 0.882 (d, J=6.8 Hz, 3H, CH₃-21), 0.845 (d, J=6.8 Hz, 3H, CH₃), 0.841 (d, J=6.8 Hz, 3H, CH₃), 0.674 (s, 3H, CH₃-18); ¹³C NMR (CDCl₃) δ 208.22 (C-5), 202.42 (C-6), 147.46 (C-3), 128.44 (C-4), 56.08 (C-17), 54.96 (C-14), 47.80 (C-10), 45.05 (C-7), 42.33 (C-13), 42.04 (C-9), 39.73 (C-12), 39.43 (C-24), 35.93 (C-22), 35.71 (C-20), 35.42 (C-1), 33.77 (C-8), 27.97 (C-25), 27.67 (C-16), 25.22 (C-15), 24.67 (C-2), 23.71 (C-23), 23.27 (C-11), 22.77 (C-27), 22.51 (C-26), 18.54 (C-21), 17.71 (C-19), 11.48 (C-18). HRMALDITOFMS calcd for C₂₇H₄₅O₂ (M+H)⁺ 401.3414, found 401.3404.

2,4-Dinitrophenylhydrazone of 5-oxo-5,6-secocholest-3-en-6-al (6b) 2,4-Dinitrophenylhydrazine (45 mg, 0.23 mmol) was added to a solution of ketoaldehyde 6a (80 mg, 0.2 mmol) and p-toluenesulfonic acid (1 mg, 0.0052 mmol)in acetonitrile (10 ml). The reaction mixture was stirred for 2 h at room temperature and evaporated to dryness in vacuo. The residue was dissolved in methylene chloride (10 ml) and was washed with water (3×20 ml). The combined organic fractions were dried over sodium sulfate and evaporated to dryness in vacuo. The residue was purified by silica gel chromatography [ethyl acetate-hexane (15:85)] to give the title compound 6b as a yellow solid (70 mg, 60%):

trans-6b ¹H NMR (CDCl₃) shows δ 10.958 (s, 1H, NH), 9.104 (d, J=2.4 Hz, 1H, H-3′), 8.288 (dd, J=9.8, 2.8 Hz, 1H, H-5′), 7.896 (d, J=9.6 Hz, 1H, H-6′), 7.337 (dd, J=5.6, 5.6 Hz, 1H, H-6), 6.771 (m, 1H, H-3), 5.822 (d, J=10 Hz, 1-H, H-4), 2.600 (ddd, J=16.4, 4.8, 4.8 Hz, 1H, H-7), 1.139 (s, 3H, CH₃-19), 0.897 (d, J=6.4 Hz, 3H, CH₃-21), 0.840 (d, J=6.8 Hz, 3H, CH₃), 0.837 (d, J=6.8 Hz, 3H, CH₃), 0.703 (s, 3H, CH₃-18); ¹³C NMR (CDCl₃) δ 207.78 (C-5), 151.17 (C-6), 147.69 (C-3), 145.00 (C-1′), 137.61 (C-4′), 129.97 (C-5′), 128.52 (C-2′), 128.38 (C-4), 123.48 (C-3′), 116.46 (C-6′), 56.05 (C-17), 54.68 (C-14), 47.87 (C-10), 42.30 (C-13), 41.69 (C-9), 39.72 (C-12), 39.37 (C-24), 36.35 (C-8), 35.91 (C-22), 35.66 (C-20), 35.34 (C-1), 32.84 (C-7), 27.93 (C-25), 27.73 (C-16), 24.93 (C-15), 24.68 (C-2), 23.69 (C-23), 23.24 (C-11), 22.74 (C-27), 22.48 (C-26), 18.52 (C-21), 17.81 (C-19), 11.58 (C-18); HRMALDITOFMS calcd for C₃₃H₄₈N₄O₅Na (M+Na)⁺ 603.3517, found 603.3523. HPLC-MS detection: R_{T} 18.3 min; [M−H]⁻ 579; λₘₐₓ 360 nm, ε 2.29±0.23×10⁴ M⁻¹cm⁻¹.

5β-Hydroxy-B-norcholest-3-ene-6β-carboxaldehyde (7a). This compound was synthesized as generally described in P. Yates, S. Stiveer, Can. J. Chem. 66, 1209 (1988). Sodium methoxide in methanol (0.5 M, 0.16 mmol) was added dropwise to a solution of ketoaldehyde 4a (50 mg, 0.125 mmol) in anhydrous methanol (10 ml) under an argon atmosphere at room temperature. After 30 min, the methanol was removed in vacuo, and the residue was dissolved in dichloromethane (20 ml) washed with water (3×20 ml). The combined organic fractions were dried over sodium sulfate, and evaporated in vacuo. The residue was purified by silica gel chromatography [ethyl acetate-hexane (1:9)] to give the title aldehyde 7a as a colorless oil (16 mg, 32%):

¹H NMR (CDCl₃) δ 9.703 (d, J=3.2, 1H, CHO), 5.716 (m, 2H, H-3 and H-4), 2.398 (dd, J=9.6, 3.6 Hz, 1H, H-6), 0.953 (s, 3H, CH₃-19), 0.904 (d, J=6.4 Hz, 3H, CH₃-21), 0.854 (d, J=6.4 Hz, 3H, CH₃), 0.849 (d, J=6.4 Hz, 3H, CH₃), 0.706 (s, 3H, CH₃-18); ¹³C NMR (CDCl₃) δ 204.41 (C-7), 134.21 (C-3), 126.66 (C-4), 81.44 (C-5), 64.49 (C-9), 55.86 (C-14), 55.55 (C-17), 48.44 (C-6), 45.12 (C-10), 44.47 (C-13), 39.92 (C-8), 39.45 (C-12), 39.40 (C-24), 36.16 (C-22), 35.57 (C-20), 29.06 (C-1), 28.31 (C-16), 27.98 (C-25), 24.73 (C-15), 23.76 (C-23), 22.78 (C-27), 22.53 (C-26), 21.69 (C-2), 21.24 (C-11), 18.74 (C-21), 18.44 (C-19), 12.37 (C-18); HRMALDITOFMS calcd for C₂₇H₄₄O₂Na (M+Na)⁺ 423.3233, found 423.3240.

2,4-Dinitrophenylhydrazone of 5β-hydroxy-B-norcholest-3-ene-6β-carboxaldehyde (7b): 2,4-Dinitrophenylhydrazine (8 mg, 0.041 mmol) and p-toluenesulfonic acid (1 mg, 5.2 μmol) were added to a solution of aldehyde 7a (15 mg, 0.037 mmol) in acetonitrile (5 ml). The reaction mixture was stirred 2 h at room temperature, evaporated under vacuum and diluted with methylene chloride (10 ml). The organic layer was washed with water (3×20 ml), dried over sodium sulfate and evaporated to dryness. The residue purified by silica gel chromatography [ethyl acetate-hexane (1:9)] to give hydrazone 7b as a yellow solid (9 mg, 41%): ¹H NMR (CDCl₃) trans-7b 11.060 (s, 1H, NH), 9.119 (d, J=2.8 Hz, 1H, H-3′), 8.291 (dd, J=9.2, 2.0 Hz, 1H, H-5′), 7.930 (d, J=9.6 Hz, 1H, H-6′), 7.546 (d, J=7.2 Hz, 1H, H-7), 5.761 (ddd, J=10.2, 4.4, 2.0 Hz, 1H, H-3), 5.705 (d, J=9.6 Hz, 1H, H-4), 2.485 (dd, J=10.4, 7.6 Hz, 1H, H-6), 0.977 (s, 3H, CH₃-19), 0.917 (d, J=6.4 Hz, 3H, CH₃-21), 0.848 (d, J=6.8 Hz, 3H, CH₃), 0.844 (d, J=6.4 Hz, 3H, CH₃), 0.707 (s, 3H, CH₃-18); ¹H—¹H ROESY NMR significant correlations (H₄-H₆), (H₆-H₇), (H₇-H₈), (H₇-H₁₉), missing correlations (H₃-H₁₉), (H₄-H₇), (H₄-H₁₉), (H₆-H₁₉); ¹³C NMR (CDCl₃) δ 154.62 (C-7), 145.09 (C-1′), 137.59 (C-4′), 133.89 (C-3), 129.94 (C-5′), 128.68 (C-2′), 127.12 (C-4), 123.57 (C-3′), 116.42 (C-6′), 80.91 (C-5), 56.83 (C-9), 56.07 (C-14), 55.39 (C-17), 49.58 (C-6), 45.00 (C-10), 44.58 (C-13), 42.50 (C-8), 39.44 (C-12), 39.44 (C-24), 36.17 (C-22), 35.54 (C-20), 30.46 (C-1), 28.53 (C-16), 27.98 (C-25), 24.91 (C-15), 23.74 (C-23), 22.77 (C-27), 22.52 (C-26), 21.79 (C-2), 21.31 (C-11), 18.76 (C-21), 18.76 (C-19), 12.34 (C-18). HPLC-MS detection: R_{T} 18.3 min; [M−H]⁻ 579; λₘₐₓ 364 nm, ε 2.32±0.17×10⁴ M⁻¹cm⁻¹.

3β-Hydroxy-B-norcholest-5-ene-6-carboxaldehyde (8a) A solution of aldehyde 5a (50 mg, 0.12 mmol) and phosphoric acid (85%, 5 ml) in acetonitrile-methylene chloride (1:1, 4 ml) was heated under reflux for 30 min. The reaction mixture was evaporated in vacuo, diluted with methylene chloride (50 ml), washed with water (3×20 ml). The organic layer was dried over sodium sulfate and evaporated under vacuum. The residue was purified by liquid chromatography on silica gel with ethyl acetate-hexane (1:4) to give the title aldehyde 12 mg (25%) of α,β-unsaturated aldehyde 8a: The ¹H NMR (CDCl₃) of 8a shows δ 9.958 (s, 1H, CHO), 3.711 (tt, J=10.8, 4.5 Hz, 1H, H-3), 3.475 (dd, J=14.1, 4.8, 1H, H-4), 2.563 (dd, J=11.0, 11.0 Hz, 1H, H-8), 0.953 (s, 3H, CH₃-19), 0.941 (d, J=6.9 Hz, 3H, CH₃-21), 0.881 (d, J=6.6 Hz, 3H, CH₃), 0.876 (d, J=6.6 Hz, 3H, CH₃), 0.746 (s, 3H, CH₃-18); ¹³C NMR (CDCl₃) δ 189.44 (C-7), 168.74 (C-5), 139.21 (C-6), 70.88 (C-3), 60.16 (C-9), 55.40 (C-17), 54.48 (C-14), 46.35 (C-10), 46.19 (C-8), 45.27 (C-13), 39.86 (C-12), 39.55 (C-24), 36.26 (C-4), 36.22 (C-22), 35.64 (C-20), 33.93 (C-1), 31.32 (C-2), 28.62 (C-16), 28.09 (C-25), 26.65 (C-15), 24.00 (C-23), 22.90 (C-27), 22.64 (C-26), 20.80 (C-11), 19.02 (C-21), 15.73 (C-19), 12.59 (C-18); HRMS calcd for C₂₇H₄₄O₂Na (M+Na)⁺ 423.3233, found 423.3239.

B-norcholest-3,5-diene-6-carboxaldehyde 12a a white solid (27 mg, 60%), was obtained as a side-product from this reaction: The ¹H NMR (CDCl₃) δ 10.017 (s, 1H, CHO), 6.919 (d, J=10.2 Hz, 1H, H-4), 6.225 (m, 1H, H-3), 2.675 (dd, J=10.8, 10.8 Hz, 1H, H-8), 0.950 (d, J=6.9 Hz, 3H, CH₃-21), 0.914 (s, 3H, CH₃-19), 0.882 (d, J=6.8 Hz, 3H, CH₃), 0.877 (d, J=6.8 Hz, 3H, CH₃), 0.769 (s, 3H, CH₃-18); ¹³C NMR (CDCl₃) δ 189.41 (C-7), 163.33 (C-5), 138.18 (C-6), 135.75 (C-3), 120.68 (C-4), 59.54 (C-9), 55.41 (C-17), 54.30 (C-14), 45.47 (C-8), 45.08 (C-10), 44.72 (C-13), 39.79 (C-12), 39.55 (C-24), 36.27 (C-22), 35.65 (C-20), 34.18 (C-1), 28.62 (C-16), 28.09 (C-25), 26.72 (C-15), 24.00 (C-23), 23.96 (C-2), 22.90 (C-27), 22.64 (C-26), 20.72 (C-11), 19.03 (C-21), 14.87 (C-19), 12.62 (C-18); HRMALDITOFMS calcd for C₂₇H₄₃O (M+H)⁺ 383.3308, found 383.3309.

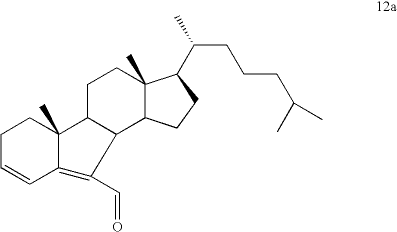

Aldolization of ketoaldehyde 4a with amino acids. In a typical procedure, ketoaldehyde 4a (2 mg, 4.8 μmol) was dissolved in DMSO-d₆ (800 μl) and D₂O (80 μl) in an NMR tube. To this solution was added 1 equivalent of either: a) L-proline, b) glycine, c) L-lysine hydrochloride or d) L-lysine ethyl ester dihydrochloride. At time points the samples were analyzed by ¹H NMR. The reaction was followed routinely by monitoring changes in a number of resonances in the ¹H NMR (DMSO-d₆) ¹H NMR 5a shows δ 9.527 (d, J=3.2 Hz, 1H, CHO), 3.876 (m, 1H, H-3), 0.860 (d, J=6.4 Hz, 3H, CH₃-21), 0.772 (d, J=6.8 Hz, 3H, CH₃), 0.767 (d, J=6.8 Hz, 3H, CH₃), 0.771 (s, 3H, CH₃-19), 0.642 (s, 3H, CH₃-18). ¹H NMR 4a shows δ 9.518 (s, 1H, CHO), 4.223 (m, 1H, H-3), 2.994 (dd, J=12.8, 4.0 Hz, 1H, H-4e), 0.858 (d, J=6.8 Hz, 3H, CH₃), 0.842 (s, 3H, CH₃-19), 0.811 (d, J=6.8 Hz, 3H, CH₃), 0.807 (d, J=6.4 Hz, 3H, CH₃-21), 0.615 (s, 3H, CH₃-18). Under these conditions, no aldolization of 4a occurs in DMSO-d₆ (800 μl) and D₂O (80 μl).

Aldolization of secoketoaldehyde 4a with atherosclerotic artery and blood fractions. In a typical procedure, ketoaldehyde 4a (5 mg, 0.0012 mmol) was dissolved in DMSO-d₆ (800 μl) and D₂O (80 μl). To this solution was added either a) atherosclerotic artery (2.1 mg) that had been homogenized in PBS (1 ml) in a tissue homogenizer and then lyophilized to dryness, b) lyophilized human blood (1 ml), c) lyophilized human plasma (1 ml) or d) PBS lyophilized (1 ml). At time points samples were removed and analyzed by ¹H NMR vide supra. Under these conditions no aldolization of 4a occurred in the presence of lyophilized PBS.

Biological Investigations with 4a and 5a

Some oxysterols have been described that are generated by oxidation of cholesterol in vivo. E. Lund, I. Björkhem, Acc. Chem. Res. 28, 241 (1995). Moreover, an analogue of 5a that differs structurally only in the cholestan side chain has been isolated from the marine sponge Stelletta hiwasaensis as part of a general screen for cytotoxic natural products. T. Miyamoto, K. Kodama, Y. Aramaki, R. Higuchi, R. W. M. Van Soest, Tetrahedron Lett. 42, 6349 (2001); B. Liu, Z. Weishan, Tetrahedron Lett. 43, 4187 (2002). However, derivatives where the steroid nucleus is disrupted, as in sterols 4a and 5a, have not previously been reported in humans.

Cytotoxicity assays. WI-L2 human B-lymphocyte line, HAAE-1 human abdominal aortic endothelial line, MH-S murine alveolar macrophage line, and J774A.1 murine tissue macrophage line were obtained from the ATCC. Human aortic endothelial cells (HAEC) and human vascular smooth muscle cells (VSMS) were obtained from Cambrex Bio Science. Jurkat E6-1T-lymphocytes were kindly provided by Dr. J. Kaye (The Scripps Research Institute). Cells were cultured in ATCC-recommended media with 10% fetal calf serum. Cells were incubated in a controlled atmosphere at 37° C., with 5 or 7% CO₂. For lactate dehydrogenase (LDH) release assays, adherent cells were harvested either by addition of 0.05% trypsin/EDTA or by scraping. The cells obtained were seeded onto 96-well microtiter plates (25,000 cells/well) and allowed to recover for 24-48 h. Cells were washed gently and media replaced with fresh media containing 5% fetal calf serum. Duplicate or greater numbers of cell samples were treated with either 3, 4a or 5a (0-100 μM) for 18 h. Cytotoxicity was then determined by measuring lactate dehydrogenase (LDH) release from cells in culture. Briefly, LDH activity was measured in the cell supernatant using the CytoTox 96 Non-Radioactive Cytotoxicity Assay (Promega, USA) of cells cultured in 96-well plates at the end of the treatment period with either ketoaldehyde 4a, aldol 5a, or cholesterol 3. 100% Cytotoxicity was defined as the maximum amount of LDH released by dead cells as shown by trypan blue exclusion, or the highest amount of LDH detected upon lysis of cells by 0.9% Triton X-100. The IC₅₀ values were determined by comparison of the raw duplicate data for concentration versus cytotoxicity (%) to non-linear regression analysis (Hill plot) using Graphpad v3.0 software for Macintosh.

Lipid-loading assay (foam cellformation). J774.1 macrophages were incubated in ATCC-recommended media containing 10% fetal bovine serum under a controlled atmosphere of 5 or 7% CO₂ at 37° C., in 8-well chamber slides. Cells were then incubated for 72 h in the same media containing the antioxidants 2,6-di-tert-butyl-4-methylphenol toluene (100 μM), diethylenetriamine-pentaacetic acid (100 μM) and either LDL (100 μg/mL), LDL (100 μg/mL) and 4a (20 μM) or LDL (100 μg/mL) and 5a (20 μM). At termination, cells were washed twice with PBS (pH 7.4). The cells were then fixed with 6% (v/v) paraformaldehyde in PBS for 30 minutes, rinsed with propylene glycol for 2 minutes and lipids were stained with 5 mg/ml Oil Red O for 8 minutes. The cells were counterstained with Harris' hematoxylin for 45 seconds, and background staining was removed with 6% paraformaldehyde followed by washing once in PBS and once in tap water. Cover slips were mounted onto the glass slides using glycerol and the slide preparations were examined by light microscopy. The number of lipid-laden cells was scored out of a total of at least 100 cells counted in a single field in each slide, and expressed as a percentage of total cells. Photographs were taken at 100× magnification.

Circular dichroism. Circular dichroism (CD) spectra of LDL (100 μg/ml), LDL (100 μg/ml) and 4a (10 μM), and LDL (100 μg/ml) and 5a (10 μM) in PBS (pH 7.4 with 1% isopropanol) were recorded at 37° C. on an Aviv spectropolarimeter, in thermostatically controlled (±0.1° C.) 0.1 cm quartz cuvettes. Spectra were recorded in the peptidic range (200-260 nm). To increase the signal-to-noise ratio, multiple spectra (three) were averaged for each measurement. The deconvolution of the molar elipticity spectra for each measurement was performed using the CDPro suite of software (by Narasimha Sreerama from Colorado State University) on a Dell PC.

### EXAMPLE 2

### Atherosclerotic Plaques Generate Ozone and Cholesterol Ozonolysis Products

Using the methods described hereinabove, this Example shows that atherosclerotic tissue, obtained by carotid endarterectomy from 15 human patients (n=15), can produce ozone detectable by reaction with indigo carmine 1.

Bleaching of Indigo Carmine by Ozone Produced by Atherosclerotic Plaques

The inventors have previously that when antibody-coated white cells were treated with the protein kinase C activator, 4-β-phorbol 12-myristate 13-acetate (PMA), in a solution of indigo carmine 1 (a chemical trap for ozone), the visible absorbance of indigo carmine 1 was bleached and indigo carmine 1 was converted into isatin sulfonic acid 2. See, e.g., P. Wentworth Jr. et al., Science 298, 2195 (2002); B. M. Babior, C. Takeuchi, J. Ruedi, A. Guitierrez, P. Wentworth Jr., Proc. Natl. Acad. Sci. U.S.A. 100, 3920 (2003); P. Wentworth Jr. et al., Proc. Natl. Acad. Sci. U.S.A. 100, 1490 (2003). The structure of isatin sulfonic acid 2 is provided in FIG. 1A. When these experiments were performed in H₂¹⁸O (>95% ¹⁸O), isotope incorporation into the lactam carbonyl of isatin sulfonic acid 2 was observed. Id. This procedure distinguished ozone and ¹O₂* from other oxidants that may also oxidize indigo carmine 1, because among the oxidants thought to be associated with inflammation, only ozone oxidatively cleaves the double bond of indigo carmine 1 with isotope incorporation (from in H₂¹⁸O) into the lactam carbonyl of isatin sulfonic acid 2 (see id. and FIG. 1A).

As described in Example 1, plaque material was obtained by carotid endarterectomy from 15 human patients believed to have problematic atherosclerosis. Each plaque was split into two equal portions (about 50 mg wet weight suspended in 1 mL of PBS). Each portion of plaque material was added to a solution of indigo carmine 1 (200 μM) and bovine catalase (50 μg/mL) in phosphate buffered saline (PBS, pH 7.4, 10 mM phosphate buffer, 150 mM NaCl) (1 mL). The analysis was initiated by addition of DMSO (10 μL) or phorbol myristate (PMA, 10 μL, 20 μg/mL) in DMSO to one or the other aliquot of suspended plaque materials.

Bleaching of the visible absorbance of 1 was observed in 14 of the 15 plaque samples upon PMA addition (FIG. 1B). This bleaching was accompanied by formation of isatin sulfonic acid 2 as determined by reversed-phase HPLC analysis (FIG. 1A and C). The amount of isatin sulfonic acid 2 formed varied from 1.0 to 262.1 nmol/mg depending upon the plaque isolate tested. The mean amount of isatin sulfonic acid 2 generated by the different isolates was 72.62±21.69 nmol/mg.

When the PMA activation of suspended plaque material was performed in H₂¹⁸O-containing PBS (>95% 18O) (n=2) with indigo carmine 1 (200 μM), approximately 40% of the lactam carbonyl oxygen of indigo carmine 1 incorporated ¹⁸O, as shown by the relative intensities of the [M−H]⁻ 228 and 230 mass fragment peaks in the mass spectrum of the isolated cleaved product isatin sulfonic acid 2 (FIG. 1D).

These studies with indigo carmine 1 indicate that ozone was produced by activated atherosclerotic plaque material.

Ozonolysis Products of Cholesterol

One of the major lipids present in atherosclerotic plaques is cholesterol 3. D. M. Small, Arteriosclerosis 8, 103 (1988). In a chemical model study, workers have shown that amongst a panel of oxidants such as, ³O₂, ¹O₂*, •O₂⁻, O₂²⁻, hydroxyl radical, O₃ and •O₂₊ and ozone O₃, only ozone cleaves the Δ^{5,6} double bond of cholesterol 3 to yield the 5,6-secosterol 4a (FIG. 2A). This observation is in agreement with other chemical reports, which also indicate that the 5,6-secosterol 4a is the principle product of cholesterol 3 ozonolysis. Gumulka et al. J. Am. Chem. Soc. 105, 1972 (1983); Jaworski et al., J. Org. Chem 53, 545 (1988); Paryzek et al., J. Chem. Soc. Perkin Trans. 1, 1222 (1990); Comforth et al., Biochem. J. 54, 590 (1953).

Further experiments were therefore directed toward detecting and identifying whether the 5,6-secosterol 4a or other ozonolysis products of cholesterol were present in atherosclerotic plaques. Human atherosclerotic plaques of 14 patients (n=14) were therefore searched for the presence of the 5,6-secosterol 4a both prior to and after activation with PMA.

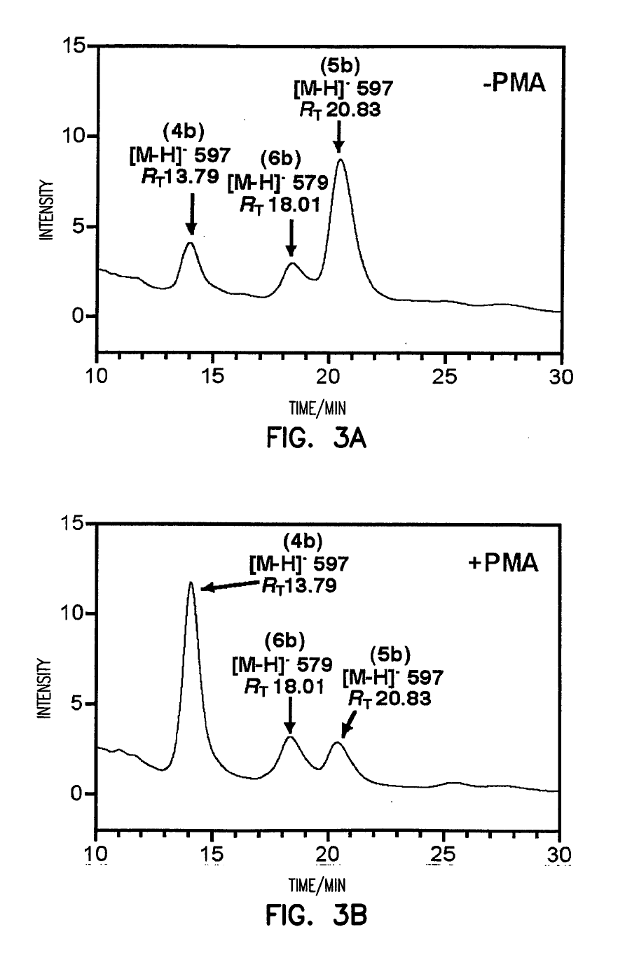

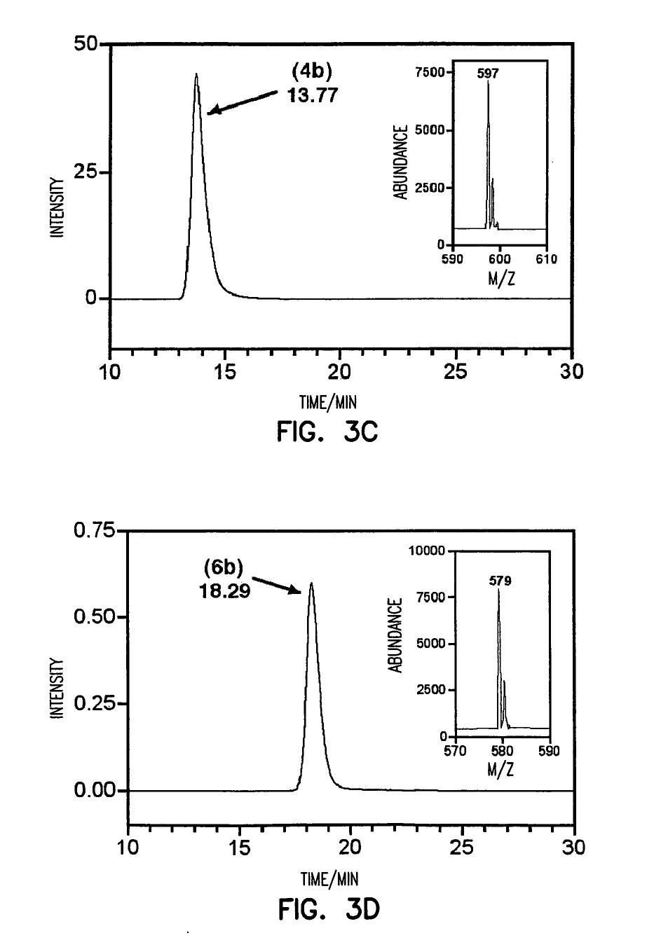

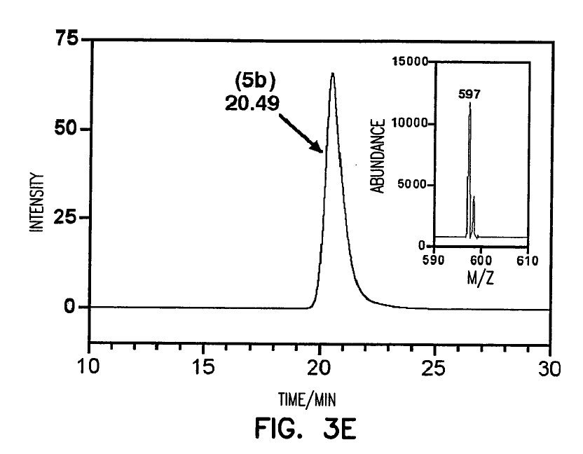

A modification of the analytical procedure developed by Pryor and colleagues was used for these studies. See K. Wang, E. Bernúdez, W. A. Pryor, Steroids 58, 225 (1993). This modified process involved extraction of a suspension of the homogenized plaque material (about 50 mg wet weight) in PBS (1 mL, pH 7), with an organic solvent (methylene chloride, 3×5 mL) followed by treatment of the organic fraction with an ethanolic solution of 2,4-dinitrophenylhydrazine hydrochloride (DNPH HCl) (2 mM in ethanol at pH 6.5) for 2 h at room temperature. This reaction mixture was analyzed by HPLC (direct injection, u.v. detection at 360 nm) and in-line negative ion electrospray mass-spectroscopy for the presence of 4b, the 2,4-dinitrophenylhydrazone derivative of the ozonolysis product 4a (FIG. 3). The hydrazone 4b was detected in 11 of the 14 unactivated plaques extracts (between 6.8 and 61.3 pmol/mg of plaque) and in all activated plaque extracts (between 1.4 and 200.6 pmol/mg). Furthermore, the amount of 4a, as judged by the mean amount of 4b, in the plaque materials significantly increased upon activation with PMA. In particular, when no PMA was used, the mean amount of 4b was 18.7±5.7 pmol/mg. In contrast, when PMA was added, the mean amount of 4b was 42.5±13.6 pmol/mg (n=14, p<0.05) (FIG. 3A-B).

In addition to 4b, two other major hydrazone peaks were observed during HPLC analysis of plaque extracts. The first peak had a R_{T}˜20.5 min and [M−H]⁻=597 and the second had a R_{T}˜18.0 min and [M−H]⁻ 579 (FIGS. 3A,B). The hydrazone 4b was readily distinguishable from these peaks because it had a retention time of about 13.8 min (R_{T}˜13.8 min, [M−H]⁻ 597) (FIGS. 3A,B). By comparison with authentic samples, the peak with a R_{T}˜20.8 min was determined to be the hydrazone derivative 5b of the aldol condensation product 5a (FIGS. 2 and 3E). In chemical model studies, Pryor had previously noted that a major side-product of the hydrazine derivatization of 4a was the hydrazone derivative 5b of the aldol condensation product 5a, and the relative amount of which was a function of both acid concentration and reaction time. K. Wang, E. Bermúdez, W. A. Pryor, Steroids 58, 225 (1993).

The extent of conversion of 4a into 5b under the conditions of derivatization employed was about 20%, over the range of 4a concentrations tested (5 to 100 μM). However, more than 20% conversion was often observed. The measured amount of 5a that exceeded 20% of the 4a present in the same plaque sample likely arose from ozonolysis of 3 followed by aldolization.

Many biochemical constituents that contain amino or carboxylate groups may catalyze aldolization reactions. Such components are present in plaques and blood, and may facilitate the conversion of 4a into 5a. Further experimentation indicated that the following amino acids and materials facilitated conversion of 4a into 5a: L-Pro (2 h, complete conversion), Gly (24 h, complete conversion), L-Lys.HCl (24 h, complete conversion), L-Lys(OEt).2HCl (100 h, 62% conversion) as well as extracts from atheromatous arteries (22 h, complete conversion), whole blood (15 h, complete conversion), plasma (15 h, complete conversion) and serum (15 h, complete conversion). All such agents accelerated the conversion of 4a into 5a relative to the rate of the background reaction.

As described above, the amount of ketoaldehyde 4a within the plaques increased upon PMA activation. However, the effect of PMA on formation of 5a was less clear. In some cases, the levels of 5a increased after PMA activation (FIG. 5B, patients F and H) while in other cases the levels of 5a decreased after PMA activation (FIG. 5B, patients C, G and N).

A number of carbonyl-containing steroid-derivatives 6a-9a whose 2,4-dinitrophenylhydrazone derivatives had a peak [M−H]⁻ of 579 in the mass spectrum (FIG. 2B) were synthesized and analyzed to assist in the identification of the peak at 18 min [M−H]⁻ 579 (FIGS. 3A,B). By comparison to HPLC coinjection, negative electrospray mass-spectrometry and u.v. spectra of authentic samples, the peak at ˜18 min was determined to be 6b, the hydrazone derivative of 6a, and the A-ring dehydration product of 4a (FIG. 3D). The extent of conversion of 4a into 6b was investigated under the standard conditions selected for derivatization. This extent of conversion was consistently found to be less than 2% over the range of 4a concentrations tested (5 to 100 μM). These data indicate that the amount of 6a present within a plaque extract that exceeded 2% of the amount of ketoaldehyde 4a within that extract, was present prior to derivatization and arose from ozonolysis product 4a by β-elimination of water.

In addition to the three major hydrazone products 4b-6b, another product 7b, was detected and determined to be the hydrazone derivative of 7a, and the A-ring dehydration product of 5a. This product (7b) was present in trace amounts (<5 pmol/mg) in several plaque extracts and had a retention time of about 26 min ([M−H]⁻ 579, FIG. 4). However, the amount of 7b in the plaque extracts was approaching the detection 1 mit of the HPLC assay employed, and a complete investigation as to the presence or absence of this compound in all the plaque samples has not yet been performed.

The experimental evidence that activated plaque material oxidatively cleaves the double bond of indigo carmine 1 with the chemical signature of ozone and that the Δ^{5,6}-double bond of cholesterol is cleaved by a pathway that, according to known chemistry, is unique to ozone gives compelling evidence that atherosclerotic plaques can generate ozone. Furthermore, since these unique ozone oxidation products of cholesterol are also present prior to plaque activation it is likely that ozone is also generated during the evolution of the atherosclerotic plaque.

It is well established that exogenously administered ozone is pro-inflammatory in vivo, via activation of interleukin (IL)-1α, IL-8, interferon (IFN)-γ, platelet aggregating factor (PAF), growth-related oncogene (Gro)-α, nuclear factor (NF)-κB and tumor necrosis factor (TNF)-α. In addition to these generally known effects of ozone in inflammation, there are circumstances unique to the atherosclerotic plaque that may increase the pathological role of endogenously-generated ozone for the initiation and perpetuation of disease when it is produced at this site. The ozonolysis of cholesterol may be unique to the plaque because it is only at this site where the requisite high concentration of ozone and cholesterol occur in the absence of other reactive substances that could trap any generated ozone.

In so far as atherosclerotic arteries contain both antibodies and a ¹O₂* generating system, in the form of activated macrophages and myeloperoxidase, it is likely that atherosclerotic lesions can generate O₃ via the antibody-catalyzed water oxidation pathway. Indeed, the observation that the Δ^{5,6}-double bond of 3 is cleaved to give 4a is further evidence for the production of ozone by antibody catalysis in inflammation. Many oxysterols are known to be generated by oxidation of cholesterol in vivo and an analogue of 5a that differs structurally only in the cholestan side chain has been isolated from the marine sponge Stelletta hiwasaensis as part of a general screen for cytotoxic natural products. T. Miyamoto, K. Kodama, Y. Aramaki, R. Higuchi, R. W. M. Van Soest, Tetrahedron Letter 42, 6349 (2001); B. Liu, Z. Weishan, Tetrahedron Lett. 43, 4187 (2002). However, derivatives where the steroid nucleus has been disrupted, as in sterols 4a-6a, have to our knowledge never before been reported in man. Therefore it is important to instigate a search for other such steroids and their derivatives and investigate their biological functions.

### EXAMPLE 3

### Cholesterol Ozonolysis Products Exist in the Bloodstream of Atherosclerosis Patients

The inventors have previously shown that ozone is generated during the antibody-catalyzed water oxidation pathway and that ozone, as a powerful oxidant, could play a role in inflammation. P. Wentworth Jr. et al., Science 298, 2195 (2002); B. M. Babior, C. Takeuchi, J. Ruedi, A. Guitierrez, P. Wentworth Jr., Proc. Natl. Acad. Sci. U.S.A. 100, 3920 (2003); P. Wentworth Jr. et al., Proc. Natl. Acad. Sci. U.S.A. 100, 1490 (2003).

Inflammation is thought to be a factor in the pathogenesis of atherosclerosis. R. Ross, New Engl. J. Med. 340, 115 (1999); G. K. Hansson, P. Libby, U. Schönbeck, Z.-Q. Yan, Circ. Res. 91, 281 (2002). However, prior to the invention, no specific non-invasive method has been available that could distinguish inflammatory artery disease from other inflammatory processes. The unique composition of the atherosclerotic plaque, and the products released by atherosclerotic plaque materials into the bloodstream, may provide such a method. In particular, atherosclerotic lesions contain a high concentration of cholesterol. As shown herein, ozone is generated by atherosclerotic lesions and cholesterol ozonolysis products such as 4a and/or its aldolization product 5a are also generated by atherosclerotic lesions. Hence, further experiments were performed to ascertain whether such cholesterol ozonolysis products could be a marker for inflammatory artery diseases such as atherosclerosis.

Plasma samples from two cohorts of patients were analyzed for the presence of either 4a or 5a. Cohort A was comprised of patients (n=8) that had atherosclerosis disease states that were sufficiently advanced to warrant endarterectomy. Cohort B patients were randomly selected patients that had attended a general medical clinic. In six of eight patients in cohort A, aldol 5a was detected, in amounts ranging from 70-1690 nM (˜1-10 nM is the detection limit of the assay) (FIG. 5A-C). In only one of the fifteen plasma samples from cohort B was there detectable 5a. No ketoaldehyde 4a was detected in any patient's blood sample (˜1-10 nM is the detection limit of the assay). These data indicate that either 4a is converted into 5a by catalysts contained in the blood, or that components within the plasma have differential affinity for 4a and 5a.

In the past, serum analysis of “oxysterols” has been fraught with difficulty due to problems of cholesterol auto-oxidation. H. Hietter, P. Bischoff, J. P. Beck, G. Ourisson, B. Luu, Cancer Biochem. Biophys. 9, 75 (1986). However, as described herein, amongst all the oxidation products of cholesterol generated by biologically relevant oxidation of cholesterol 3, steroid derivatives 4a and 5a are unique to ozone. These studies indicate that the presence of the aldolization product 5a in plasma, detected as its DNP hydrazone derivative 5b, can be a marker for advanced arterial inflammation in atherosclerosis. Hence, the antibody-catalyzed generation of ozone may link the otherwise seemingly independent factors of cholesterol accumulation, inflammation, oxidation and cellular damage into the pathological cascade that leads to atherosclerosis

Some studies indicate that cholesterol oxidation products possess biological activities such as cytotoxicity, atherogenicity and mutagenicity. H. Hietter, P. Bischoff, J. P. Beck, G. Ourisson, B. Luu, Cancer Biochem. Biophys. 9, 75 (1986); J. L. Lorenso, M. Allorio, F. Bemini, A. Corsini, R. Fumagalli, FEBS Lett. 218, 77 (1987); A. Sevanian, A. R. Peterson, Proc. Natl. Acad. Sci. U.S.A. 81, 4198 (1984). Given that the cholesterol oxidation products 4a and 5a have never before been considered to occur in man, the effect of these compounds on key aspects of atherogenesis were further investigated as described below.

### EXAMPLE 4

### Cytotoxicity of Cholesterol Ozonolysis Products

Some cholesterol oxidation products possess biological activities such as cytotoxicity, atherogenicity and mutagenicity. In this Example, the cytotoxic effects of 4a and 5a against a variety of cell lines were analyzed.

The following cell lines were employed in this study: a human B-lymphocyte (WI-L2) described in Levy et al., Cancer 22, 517 (1968); a T-lymphocyte cell line (Jurkat E6.1) described in Weiss et al., J. Immunol. 133, 123 (1984); a vascular smooth muscle cell line (VSMC) and an abdominal aorta endothelial (HAEC) cell line described in Folkman et al., Proc. Natl. Acad. Sci. U.S.A. 76, 5217 (1979); a murine tissue macrophage (J774A.1) described in Ralph et al., J. Exp. Med. 143, 1528 (1976); and an alveolar macrophage cell line (MH-S) described in Mbawuike et al., J. Leukoc. Biol. 46, 119 (1989).

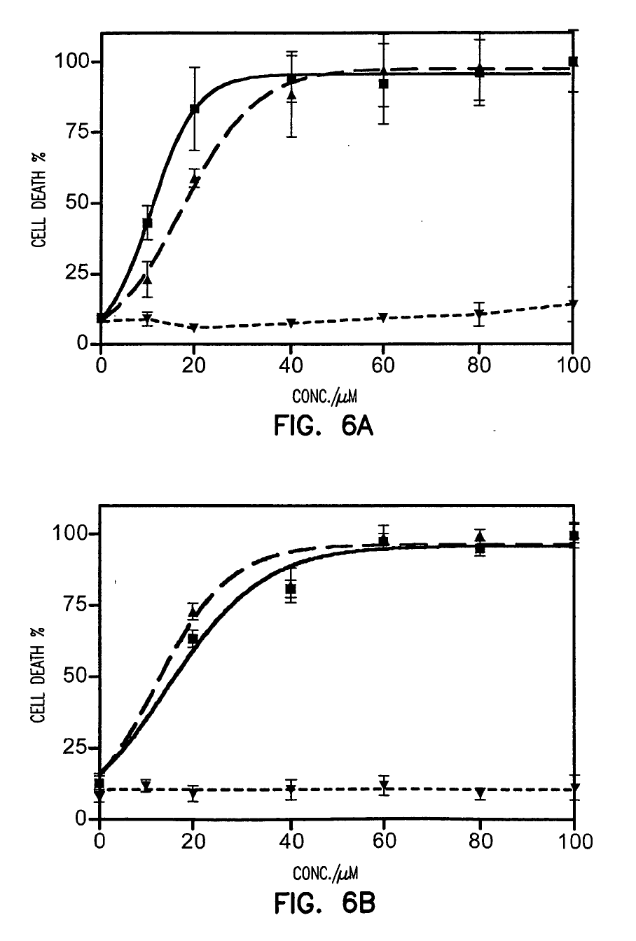

Chemically synthesized 4a and 5a are cytotoxic against a range of cell types known to be present within atherosclerotic plaque; leukocytes, vascular smooth muscle and endothelial cells. The results are shown in FIG. 6 and in Table 3.

TABLE 3

| Cell Line | IC₅₀ of 4a | IC₅₀ of 5a |
|---|---|---|
| WIL2 | 10.9 ± 1.6 μM | 17.7 ± 2.3 μM |
| Jurkat E6.1 1 | 15.5 ± 1.7 μM | 12.6 ± 1.9 μM; |
| HAEC | 24.6 ± 3.2 μM | 18.2 ± 1.9 μM |
| VSMC | 21.9 ± 2.2 μM | 29.8 ± 2.8 μM |
| J774A.1 | 15.6 ± 2.1 μM | 26.1 ± 2.8 μM |
| MH-S | 11.2 ± 1.2 μM | 13.6 ± 1.1 μM |

The IC₅₀ values of 4a and 5a are very similar against all the cells lines tested. Moreover, the cytotoxic profiles of compounds 4a and 5a against the cells lines tested were very similar. These results were surprising considering the significant structural differences between 4a and 5a. However, 4a and 5a do equilibrate with each other in a process that is facilitated by cellular components such as amino acids vide supra, 4a and 5a may be in equilibrium with each other during the time frame of the cytotoxicity assays. Hence, compounds 4a and 5a may have similar cytotoxicity in vivo.

Using similar procedures, compounds 6a, 7a, 7c, 10a, 11a and 12a have been shown by the inventors to be cytotoxic to leukocyte cell lines and the seco-ketoaldehyde 4a and its aldol adduct 5a have been shown to be cytotoxic towards neuronal cell lines. The 7c compound has the following structure.

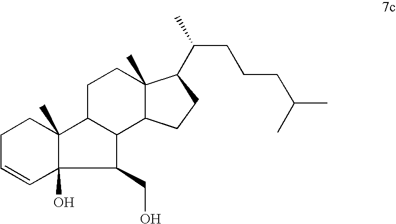

The juxtaposition of ozone and cholesterol can lead the cytotoxic steroids 4a-12a and 7c, which generated in situ may well play a role in the progression of the lesion by promoting endothelial or smooth muscle cell damage, or by triggering apoptosis of inflammatory cells within the atheroma vide supra. Ozonolysis of cholesterol within the previously described crystalline-phase of atherosclerotic plaques may contribute to plaque destabilization, which is thought to be the ultimate step prior to arterial occlusion.

### EXAMPLE 5

### Cholesterol Ozonolysis Products Promote Foam Cell Formation and Alter LDL and Apoprotein B₁₀₀ Structures

Modifications of low-density lipoprotein (LDL) that increase its atherogenicity are considered pivotal events in the development of cardiovascular disease. D. Steinberg, J. Biol. Chem. 272, 20963 (1997). For example, oxidative modifications to LDL, or to apoprotein B₁₀₀ (apoB-100, the protein component of LDL) that increase LDL uptake into macrophages via CD36 and other macrophage scavenger receptors are considered critical causative pathological events in the onset of atherosclerosis. This Example describes experiments showing that cholesterol ozonolysis products 4a and 5a can promote formation of foam cells from macrophages and modify the structure of LDL and apoB-100.

LDL (100 μg/mL) was incubated with 4a or 5a in the presence of unactivated murine macrophages (J774.1) as described in Example 1. After exposure to 4a or 5a these macrophages began lipid-loading and foam cells began to appear in the reaction vessel (FIG. 7).

Moreover, incubation of human LDL (100 μg/ml) with 4a and 5a (10 μM) led to time-dependent changes in the structure of apoB-100 as detected by circular dichroism (FIGS. 8B,C). Circular dichroism analysis of total LDL without 4a and 5a revealed that LDL secondary structure is generally stable over the duration of the experiment (48 h) (FIG. 8A). As shown in FIG. 8A, the protein content of normal LDL has a large proportion of a helical structure (˜40±2%) and smaller amounts of β structure (˜13±3%), β turn (˜20±3%) and random coil (27±2%). However, while the spectral shape of LDL incubated with 4a and 5a remains somewhat similar to native LDL (FIG. 8B and C), there is a significant loss of secondary structure, mainly a loss of a helical structure (4a˜23±5%; 5a˜20±2%) and a correspondingly higher percentage of random coil (4a˜39±2%; 5a 32±4%). Hence, the 4a and 5a cholesterol ozonolysis products appear to undermine the structural integrity of LDL.

In order to modify LDL structure, a covalent reaction may occur between the aldehyde moieties of the 4a and 5a cholesterol ozonolysis products and the ε-amino-side-groups of apoB-100 lysine residues to form Schiff-base or enamine intermediates, that are similar to compounds previously observed in a reaction between malondialdehyde and 4-hydroxynonenal with apoB-100. Steinbrecher et al., Proc. Natl. Acad. Sci. U.S.A. 81, 3883 (1984); Steinbrecher et al., Arteriosclerosis 1, 135 (1987); Fong et al., J. Lipid. Res. 28, 1466 (1987). Such Schiff-base or enamine intermediates can have a significant lifetime and may render the derivatized LDL into a form recognized by the macrophage scavenger receptors. Hence, a covalent reaction between the 4a and 5a cholesterol ozonolysis products and apoB-100-LDL may generate a derivatized apoB-100-LDL complex that is recognized and taken up at a higher rate by macrophage scavenger receptors, thereby generating the foam cells observed in FIG. 7.

The only known oxidized forms of cholesterol that contain an aldehyde component are the 4a and 5a ozonolysis products. Hence, a reaction between such cholesterol derivatives and LDL/apoB-100 may provide a here-to-fore missing link between cholesterol, foam cell formation arterial plaque formation. Detection of high levels of the 4a and 5a ozonolysis products in the bloodstream of patients may therefore provide a direct measure of the extent to which those patients suffer from atherosclerosis.

### EXAMPLE 6

### Generating Antibodies Against Cholesterol Ozonation Products

This Example describes antibodies generated against haptens having formula 13a, 14a or 15a that can react with the ozonation and hydrazone products of cholesterol. The structures of haptens having formula 13a, 14a and 15a are shown below:

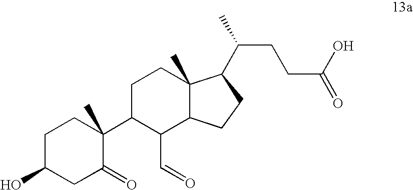

Compound 13a is 4-[4-formyl-5-(4-hydroxy-1-methyl-2-oxo-cyclohexyl)-7a-methyl-octahydro-1H-inden-1-yl]pentanoic acid. 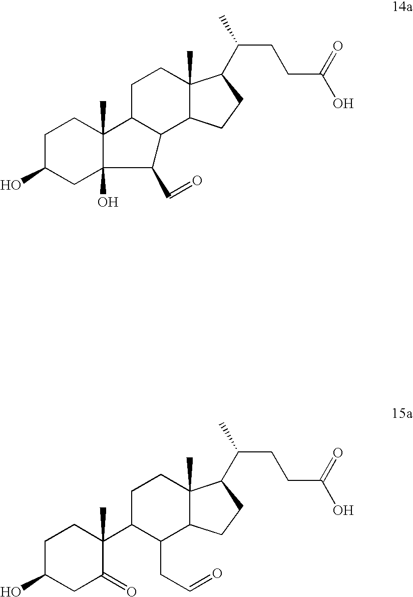
Methods

KLH conjugates of compounds 13a, 14a and 15a were prepared. Mice were immunized with these KLH conjugates by standard procedures. Spleens were removed from the mice and dispersed to obtain splenocytes as antibody-producible cells.

The splenocytes and SP2/0-Ag14 cells, ATCC CRL-1581, derived from mouse myeloma, were co-suspended in serum-free RPMI-1640 medium (pH 7.2), pre-warmed to 37° C., to give cell densities of 3×10⁴ cells/ml and 1×10⁴ cells/ml, respectively. The suspension was centrifuged to collect a precipitate. To the precipitate, 1 ml of serum-free RPMI-1640 medium containing 50 w/v % polyethylene glycol (pH 7.2) was dropped over 1 min, followed by incubating the resulting mixture at 37° C. for 1 min. Serum-free RPMI-1640 medium (pH 7.2) was further dropped to the mixture to give a final volume of 50 ml, and a precipitate was collected by centrifugation. The precipitate was suspended in HAT medium, and divided into 200 μl aliquots each for a well of 96-well microplates. The microplates were incubated at 37° C. for one week, resulting in about 1,200 types of hybridoma formed. Supernatants from the hybridomas were analyzed by immunoassay for binding to cholesterol ozonation products.

Hybridomas KA 1-11C5 and KA 1-7A6, raised against a compound having formula 15a, were deposited under the terms of the Budapest Treaty on Aug. 29, 2003 with the American Type Culture Collection (10801 University Blvd., Manassas, Va., 20110-2209 USA (ATCC)) as ATCC Accession No. ATCC Numbers PTA-5427 and PTA-5428. Hybridomas KA2-8F6 and KA2-1E9, raised against a compound having formula 14a, were deposited with the ATCC under the terms of the Budapest Treaty also on Aug. 29, 2003 as ATCC Accession No. ATCC PTA-5429 and PTA-5430.

Pools of monoclonal antibody preparations KA1-7A6:6 and KA1-11C5:6, produced against a KLH conjugate of hapten 15a, and KA2-8F6 and KA2-1E9, produced against a KLH-conjugate of hapten 14a, were generated. The binding titres of the KA1-7A6:6 and KA1-11C5:6 monoclonal antibodies elicited to 15a against ozonation products 5a and cholesterol hapten 3c were determined by ELISA assay. ELISA assays were also performed to determine the binding titres of KA2-8F6:4 and KA2-1E9:4 antibodies (elicited to ozonation product 5a) against 13b, 14b and cholesterol hapten 3c.

The structure of the cholesterol hapten 3c is provided below.

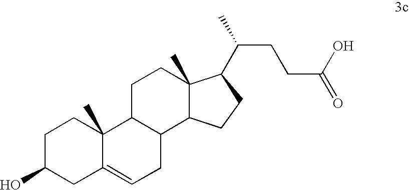

The ELISA assays were performed as follows. BSA conjugates of 13a, 14a, 3c, 13b, 14b or 15a were separately added to hi-bind 96-well microtiter plates (Fischer Biotech.) and allowed to stand overnight at 4° C. The plates were washed exhaustively with PBS and a milk solution (1% w/v in PBS, 100 μL) was added. Plates were allowed to stand at room temperature for 2 h and then washed with PBS. Cultures containing different antibody preparations were serially diluted with PBS and 50 μL of each dilution was separately added to the first well of each row. After mixing and dilution, the plates were allowed to stand overnight at 4° C. The plates were washed with PBS and a goat anti-mouse horseradish peroxidase conjugate (0.01 μg, 50 μL) was added. Plates were incubated at 37° C. for 2 h. The plates were washed and substrate solution (50 μL) 3,3′,5,5′-tetramethylbenzidine [0.1 mg in 10 mL of sodium acetate (0.1 M, pH 6.0) and hydrogen peroxide (0.01% % w/v)] was added. The plates were developed in the dark for 30 min. Sulfuric acid (1.0 M, 50 μL) was added to quench the reaction and the optical density was measured at 450 nm.

The reported titer is the serum dilution that corresponds to 50% of the maximum optical density. The data were analyzed with Graphpad Prism v. 3.0 and are reported as the mean value of at least duplicate measurements.

Results

The results of the ELISA tests are shown in Tables 4 and 5.

TABLE 4
Binding titres of anti-15a antibodies KA1-7A6:6 and KA1 11C5:6
against 15a, ozonation product 5a and cholesterol hapten 3c.

| Antibody | 15a | 5a | 3c |
|---|---|---|---|
| KA1-7A6:6 | 32,000 | 32,000 | 16,000 |
| KA1 11C5:6 | 64,000 | 64,000 | 16,000 |

*titres were measured by ELISA against a BSA conjugate of 15a, 5a and 3c. The absolute value is the dilution factor of a tissue culture supernatant solution of antibody that corresponds to 50% of maximum absorbance when bound.

As shown by Table 4, the apparent binding affinities, measured as described above, are almost identical.

TABLE 5
Binding titres of KA2-8F6:4 and KA2-1E9:4 antibodies elicited to 5a
against 15b, 14b and cholesterol hapten 3c.

| antibodies | 15b | 14b | 3c |
|---|---|---|---|
| KA2-8F6:4 | 32,000 | 32,000 | 16,000 |
| KA2-1E9:4 | 64,000 | 64,000 | 16,000 |

*titres were measured by ELISA against a BSA conjugate of 15b, 14b and cholesterol hapten 3c. The absolute value is the dilution factor of a tissue culture supernatant solution of antibody that corresponds to 50% of maximum absorbance when bound to a BSA conjugate of 13b, 15b and cholesterol hapten 3c.

These results indicate that high affinity antibody preparations can be generated against cholesterol ozonation products.

### EXAMPLE 7

### Additional Methods for Detecting Cholesterol Ozonation Products

This Example illustrates that cholesterol ozonation products can be detected by a variety of procedures, including by conjugation of the free aldehyde groups on these ozonation products to fluorescent moieties and by use of antibodies reactive with these ozonation products.

### Materials and Methods

General Methods

All reactions were performed with dry reagents, solvents, and flame-dried glassware unless otherwise stated. Starting materials were purchased and used as received from Aldrich Chemical Company, unless otherwise stated. Cholesterol-[26,26,26,27,27,27-D₆] was purchased from MEDICAL ISOTOPES, INC. Flash column chromatography was performed using silica gel 60 (230-400 mesh). Cholesterol ozonation products 4a and 5a and the 2,4-dinitrophenyl hydrazones of ozonation products 4a and 5a (4b and 5b, respectively) were synthesized as described in the previous examples. Thin layer chromatography (TLC) was performed using Merck (0.25 mm) coated silica gel Kieselgel 60 F₂₅₄ plates and visualized with para-anisaldehyde stain. ¹H NMR spectra were recorded on Bruker AMX-600 (600 MHz) spectrometer. ¹³C NMR spectra were recorded on Bruker AMX-600 (150 MHz) spectrometer. Chemical shifts are reported in parts per million (ppm) on the δ scale from an external standard.

### Synthesis of Dansyl hydrazone of 3β-hydroxy-5-oxo-5,6-secocholestan-6-al (4d)

Dansyl hydrazine (50 mg, 0.17 mmol) and p-toluenesulfonic acid (1 mg, 0.0052 mmol) was added to a solution of cholesterol ozonation product 4a (65 mg, 0.16 mmol) in acetonitrile (8 ml). The reaction mixture was stirred under an argon atmosphere for 2 h at room temperature, and evaporated to dryness in vacuo. The residue was dissolved in methylene chloride (10 ml) and washed with water (2×10 ml). The organic fraction was dried over magnesium sulfate and concentrated in vacuo. The crude yellow oil was purified by silica gel chromatography [ethyl acetate-hexane (1:1; 7:3)] to give the title compound 4d (70 mg, 68%) as a mixture of geometric isomers (cis:trans 8:92): ¹H NMR (CDCl₃) δ 9.341 (s, 1H), 8.567 (d, J=8.4 Hz, 1H), 8.358 (dd, J=7.2, 1.2 Hz, 1H), 8.290 (d, J=8.4 Hz, 1H), 7.550 (dd, J=8.4, 7.6 Hz, 1H), 7.539 (dd, J=8.4, 7.6 Hz, 1H), 7.167 (d, J=7.6 Hz, 1H), 7.000 (t, J=4.0 Hz, 0.92H trans), 6.642 (dd, J=6.8, 2.8 Hz, 0.08H cis), 4.273 (bs, 1H), 3.045 (dd, J=13.6, 3.4 Hz, 1H), 2.869 (s, 6H), 2.233 (d, J=13.6 Hz, 1H), 2.097 (dt, J=18, 4.4 Hz, 1H),1.162 (s, 3H), 0.904 (d, J=6.4 Hz, 3H), 0.899 (d, J=6.8 Hz, 3H), 0.892 (d, J=6.4 Hz, 3H), 0.513 (s, 3H); ³C NMR (CDCl₃) δ 209.66, 151.77, 149.49, 133.52, 131.20, 130.99, 129.64 (2C)*, 128.52, 123.25, 118.83, 115.25, 71.07, 56.20, 52.68, 52.56, 47.10, 45.40, 42.32, 40.81, 39.82, 39.48, 36.51, 36.05, 35.79, 34.39, 31.05, 28.02, 27.74, 27.30, 24.27, 24.13, 22.99, 22.84, 22.56, 18.53, 17.45, 11.31; HRMALDIFTMS calcd for C₃₉H₅₉N₃O₄SNa (M+Na) 688.4118, found 688.4152; R_{f} 0.43 [ethyl acetate-hexane (7:3)]. * 2C denotes that this signal is believed to correspond to two carbon signals (C₀ as per gHSQC) from the dansyl moiety.

### Synthesis of dansyl hydrazone of 3β-Hydroxy-5β-hydroxy-B-norcholestane-6β-carboxaldehyde (5c)

To a solution of cholesterol ozonation product 5a (30 mg, 0.072 mmol) in tetrahydrofuran (5 ml) was added dansyl hydrazine (25 mg, 0.08 mmol) and hydrochloric acid (conc., 0.05 ml). The white precipitate that immediately formed was dissolved by the addition of water (0.2 ml). The homogeneous reaction mixture was stirred under an argon atmosphere for 3 h at room temperature, and evaporated to dryness. The red residue was dissolved in ethyl acetate (10 ml) and washed with water (2×10 ml). The organic fraction was dried over magnesium sulfate and concentrated in vacuo. The crude yellow oil was purified first by silica gel chromatography [ethyl acetate-methylene chloride (1:4-1:1)] and then by preparative HPLC (C18 Zorbax 21.22 mm and 25 cm. 100% acetonitrile) to give the title compound 5c (14.5 mg, 30%) as a mixture of geometric isomers (cis:trans 17:83): ¹H NMR (CDCl₃) δ 8.557 (d, J=8.8 Hz, 1H), 8.372 (dd, J=7.2, 1.2 Hz, 1H), 8.300 (d, J=8.8 Hz, 1H), 8.084 (s, 1H), 7.575 (dd, J=8.8, 7.6 Hz, 1H), 7.554 (dd, J=8.8, 7.6 Hz, 1H), 7.197 (d, J=7.6 Hz, 1H), 7.057 (d, J=7.2 Hz, 0.84H trans), 6.517 (d, J=5.2 Hz, 0.16H cis), 4.229 (m, 0.17H cis), 4.004 (m, 0.83H trans), 2.905 (s, 6H), 2.379 (bm, 4H), 1.913 (dd, J=9.6, 7.2 Hz, 2H), 0.886 (d, J=6.8 Hz, 3H), 0.879 (d, J=6.4 Hz, 3H), 0.841 (d, J=6.8 Hz, 3H), 0.691 (s, 3H), 0.393 (s, 3H); ¹³C NMR (CDCl₃) δ 154.081, 133.425, 131.367, 130.912, 129.695, 128.611, 123.350, 115.121, 83.268, 70.469, 67.079, 55.773, 55.677, 55.280, 51.652, 45.429, 45.038, 44.372, 43.129, 42.443, 39.488, 36.143, 35.585, 28.580, 28.458, 27.984, 27.766, 23.850, 22.825, 22.549, 21.389, 18.659, 18.063, 12.192; HRMALDIFTMS calcd for C₃₉H₅₉N₃O₄SNa (M+Na) 688.4118, found 688.4118; R_{f}0.41 [ethyl acetate-methylene chloride (1:1)].

### Synthesis of 3β-Hydroxy-5-oxo-5,6-seco-[26,26,26,27,27,27-D₆]-cholestan-6-al (D₆-4a).

A gaseous mixture of ozone in oxygen was bubbled through a solution of D₆-cholesterol (50 mg, 0.13 mmol) in 5 mL chloroform-methanol (9:1) at −78° C. for 1 min, by which time the solution turned slightly blue. The reaction mixture was evaporated and stirred with Zn powder (40 mg, 0.61 mmol) in 2.5 mL acetic acid-water (9:1) for 3 h at room temperature. This heterogeneous mixture was diluted with methylene chloride (10 mL) and washed with water (3×5 mL) and brine (5 mL). The organic fractions were dried over magnesium sulfate and evaporated. The residue was purified using silica-gel chromatography (eluted with hexane-ethyl acetate 5:1, 3:1 and 2:1) to yield the title compound as a white solid (44 mg, 0.104 mmol), yield: 81%. ¹H NMR 600 MHz (δ, ppm, CDCl₃): 9.61 (s, 1H), 4.47 (s, 1H), 3.09 (dd, 1H, J=13.6 Hz, 4.0 Hz), 2.25-2.40 (m, 3H), 2.15-2.19 (m, 1H), 1.01 (s, 3H), 0.88 (d, 3H, J=6.1 Hz), 0.67 (s, 3H). ³C NMR 150 MHz (δ, ppm, CDCl₃): 217.5, 202.8, 71.0, 56.1, 54.2, 52.6, 46.8, 44.1, 42.5, 42.1, 39.8, 39.3, 35.9, 35.7, 34.7, 34.0, 27.8, 27.7, 27.5, 25.3, 23.7, 23.0, 18.5, 17.5, 11.5.

### Synthesis of 3β-hydroxy-5β-hydroxy-B-norcholesterol-[26, 26,26,27,27,27-D₆]-6β-carboxaldehyde (D₆-5a).

To a solution of D₆-4a (26 mg, 0.061 mmol) in acetonitrile-water (20:1, 5 mL) was added L-proline (11 mg). The reaction mixture was stirred for 2.5 h at room temperature and evaporated in vacuo. The residue was dissolved in ethyl acetate (10 mL) and washed with water (2×5 mL) and brine. The organic fraction was dried over magnesium sulfate and evaporated to leave a white solid which was analytically pure (26 mg, 0.061 mmol, yield: 100%), for NMR. ¹H NMR 600 MHz (δ, ppm, CDCl₃): 9.69 (s, 1H), 4.11 (s, 1H), 2.23 (dd, 1H, J=9.2 Hz, 3.0 Hz), 0.91 (s, 3H), 0.90 (d, 3H, J=6.6 Hz), 0.70 (s, 3H); ¹³C NMR 150 MHz (δ, ppm, CDCl₃): 204.7, 84.2, 67.3, 63.9, 56.1, 55.7, 50.4, 45.5, 44.7,44.2, 40.0, 39.7, 39.3, 36.1, 35.6, 28.3, 27.9, 27.5, 26.7, 24.5, 23.8, 21.5, 18.7, 18.4, 12.5.

Synthesis of 4-(5-(4-hydroxy-1-methyl-2-oxocyclohexyl)-7α-methyl-4-(2-oxoethyl)-octahydro-1H-inden-1-yl)pentanoic acid 15a. Ozonolysis of 3β-hydroxycholest-5-en-24-oic acid 3c, was performed as described for D₆-5a. ¹H NMR 400 MHz (δ, ppm, CDCl₃): 9.60 (s, 1H); 4.47 (s, 1H), 3.40 (dd, J=13.6 Hz, 4 Hz, 1H); 1.00 (s, 1H), 0.91 (d, J=6.4 Hz, 3H), 0.67 (s, 3H). ¹³C NMR 100 MHz (δ, ppm, CDCl₃): 218.7, 202.9, 179.8, 70.9, 55.5, 54.1, 52.5, 46.4, 44.0, 42.4, 42.1, 39.6, 35.1, 34.5, 34.0, 30.8, 30.4, 27.5, 27.3, 25.1, 22.8, 17.9, 17.4, 11.4.

Cholesterol Ozonation Product Extraction.

A modified Bligh and Dyer method was used to extract total lipids from both blood and tissue samples. See, Bligh E G, D. W. Can J Biochem Physiol 1959, 37, 911-17. Human plasma (200 μL), collected in Vacutainer tubes, containing citrate or EDTA as anticoagulant and stored at 4° C., was added to potassium dihydrogen phosphate (KH₂PO₄, 0.5 M, 300 μL) in a capped glass tube. Methanol (500 μL) was added and the sample was vortexed briefly. Chloroform (1 mL) was added and the sample was vortexed for 2 min, centrifuged at 3000 rpm for 5 min and the organic layer was removed. This process of chloroform addition, vortexing and centrifugation was repeated. The combined organic fractions were combined and evaporated in vacuo. Endarterectomy specimens were obtained from patients undergoing carotid endarterectomy for routine indications. The Scripps Green Hospital Institutional Review Board approved the human subjects protocol. Specimens were frozen and stored at −70° C. prior to analysis. For analysis, the tissue sample was allowed to warm to room temperature and was then homogenized in aqueous buffer (KH₂PO₄, 0.5M, 1-2 mL) using a tissue homogenizer (Tekmar). The homogenate was added to a solution of methanol:chloroform (1:3, 6 mL) and centrifuged at 3000 rpm for 5 min. The organic fraction was collected. Chloroform (6 mL) was added to the remaining aqueous miscible fraction and the samples were centrifuged (3000 rpm for 5 min). The combined organic fractions were then evaporated in vacuo.

Derivatization with Dansyl Hydrazine and HPLC-analysis of Extracted Cholesterol Ozonation Products.

The evaporated blood or tissue extracts vide supra are resuspended in isopropanol (200 μL) containing dansyl hydrazine (200 μM) and H₂SO₄ (100 μM) and incubated at 37° C. for 48 h. The analytical method involved HPLC analysis on a Hitachi D-7000 HPLC system connected to a Vydec C-18 RP column with an isocratic mobile phase of acetonitrile:water (90:10, 0.5 mL/min) using fluorescence detection (Excitation wavelength 360 nm, Emission wavelength 450 nm). The retention time (R_{T}) for the dansyl derivative of ozonation product 5a (5c) was about 8.1 min. The retention time for the hydrazine derivative of 5a (5b) was about 10.7 min. Concentrations were routinely determined by peak area calculations referenced to authentic standards using a Macintosh PC and Prism 4.0 software.

Gas Chromatography—Mass Spectroscopy

Evaporated specimens were reconstituted in methylene chloride to a 1 mL volume and silylated by the addition of 100 uL pyridine and 100 uL N,O-Bis(trimethylsilyl)-trifluoroacetamide with 1% trimethylchlorosilane to the concentrated plaque extract. Samples were incubated at 37° C. for 2 hours then evaporated to dryness by rotatory evaporation. Each sample was resuspended in 100 uL methylene chloride prior to analysis. 2.5 ul of sample was injected via a splitless injection (Agilent 7673 autosampler) onto an HP-5 ms column, 30 m×0.25 mm ID×0.25 um film thickness, flow rate of 1.2 ml/min, injector temp was 290° C., temperature program starts at 50° C., hold for 5 min then ramp at 20° C./min until 300° C., hold for 12 min. Mass Analysis was performed with an Agilent model 5973 inert, Scan range 50-700 m/z followed by selected ion monitoring (SIM) scans for m/z 354 and 360. MS quad temp was 150° C., with an MS source temp of 280° C.

Coupling of Hapten 15a to Carrier Proteins KLH and BSA.

1-Ethyl-3,3′-dimethylaminopropyl-carbodiimide hydrochloride (EDC, 1.5 mg, 0.008 mmol) and Sulfo N-hydroxysuccinimde (1.8 mg, 0.008 mmol) were dissolved in 0.01 mL H₂O and added to a solution of hapten (2.5 mg, 0.006 mmol) in 0.1 mL DMF. The mixture was vortexed and kept at room temperature for 24 hours before it was added to BSA (5 mg) in PBS buffer (0.9 ml, 0.05 mM at pH=7.5) at 4° C. This final mixture was kept at 4° C. for 24 hours and stored at −20° C. The reactions involved in synthesizing a KLH or BSA conjugate of compound 15a are depicted below. 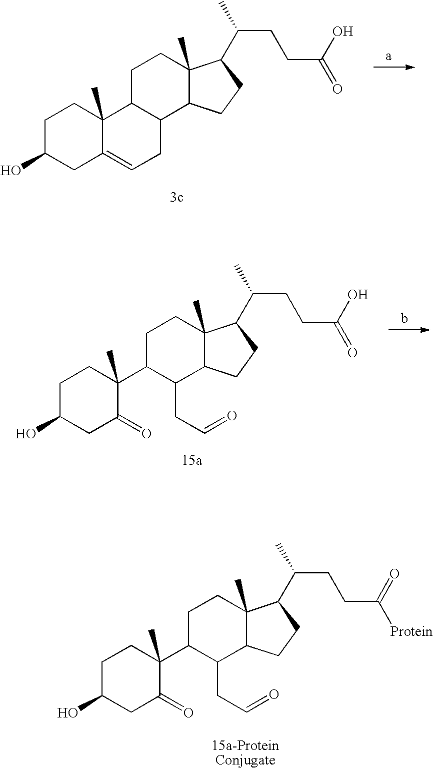
Reaction a involved ozonolysis of compound 3c with O₃/O₂ as described above. Reaction b involved treatment of compound 15a with EDC and HOBt in DMF overnight followed by incubation with BSA or KLH in phosphate buffered saline (PBS), pH 7.4.

Monoclonal antibody production was carried out by standard methods. Immunization of 8 week old 129GIX+ mice was performed with 10 ug KLH-15a conjugate in 50 uL PBS per mouse mixed with an equal volume of RIBI adjuvant injected IP every 3 days for a total of 5 immunizations. The serum titer was determined by ELISA. 30 days later, a final injection of 50 ug KLH-15a conjugate in 100 uL PBS intravenously (IV) in the lateral tail vein. Animals were sacrificed and the spleen was removed 3 days later for fusion. Spleen cells from immunized animals were mixed 5:1 with X63-Ag8.653 myeloma cells in RPMI media centrifuged, and resuspended in 1 mL PEG 1500 at 37C The PEG is diluted with 9 mL RPMI over 3 minutes and incubated at 37C for 10 minutes then centrifuged, resuspended in media and plated in 15×96 well plates. ELISA was performed to screen for antibodies that bound cholesterol ozonation product 4a or 5a but not cholesterol. Selected hybridomas were subcloned through 2 generations to guarantee monoclonality.

Preparations of Histological Sections from Ascending Aorta of ApoE Knockout Mice.

Specimens were snap frozen in liquid nitrogen. 10 micron sections were taken, and mounted on glass slides. Specimens were fixed by sequential immersion in 1:1 ethyl alcohol:diethyl ether for 20 minutes, 100% ethanol for 10 minutes, and 95% ethanol for 10 minutes. After washing in PBS, a 1:200 dilution of antibody specific for cholesterol ozonation product was applied and incubated with the tissue for 1 hour. Secondary labeling was performed with a 40:1 dilution of FITC labeled goat anti-mouse IgG (Calbiochem). Images were obtained using an optronics microfire digital camera and processed using Adobe Photoshop.

### Results

Fluorescence-detection of Dansyl Hydrazones of Cholesterol Ozonation Products.

As described in the previous Examples, cholesterol ozonation products can be detected in vivo using a modification of the analytical procedure developed in a chemical study by K. Wang, E. Bermúdez, W. A. Pryor, Steroids 58, 225 (1993). This modified process involved extraction of a suspension of the homogenized plaque material (˜50 mg wet weight) in PBS (1 mL) pH 7.4, into an organic solvent (methylene chloride, 3×5 mL) treatment of the organic soluble fraction with an ethanolic solution of 2,4-dinitrophenylhydrazine hydrochloride (DNPH HCl) (2 mM, pH 6.5) for 2 h at room temperature. This reaction mixture was analyzed by reversed-phase HPLC (direct injection, u.v. detection at 360 nm) and in-line negative ion electrospray mass-spectroscopy for the presence of 4b, the 2,4-dinitrophenylhydrazone (2,4-DNP) derivative of 4a and 5b, the 2,4-DNP derivative of 5a. This technique is both rapid and highly sensitive. However, there are a number of limitations to this assay when it is applied to biological samples. These include interference with other biologic compounds with ultraviolet absorbance at 360 nm, conversion of the 4b into 5b during the conjugation reaction, and the reduced efficiency of the conjugation reaction at low concentrations of cholesterol ozonation products.

Therefore, a new procedure was tested to ascertain whether increased assay sensitivity could be achieved. This procedure involved conjugation of cholesterol ozonation products to a hydrazine that had a fluorescent chromophore followed by fluorescence detection and HPLC analysis. The fluorescent chromophore selected was the dansyl group. The assay involved derivatization of the extracted cholesterol ozonation products with dansyl hydrazine under acidic conditions as described above. The product of dansyl hydrazine reaction with cholesterol ozonation product 4a was 4d, which is depicted below.

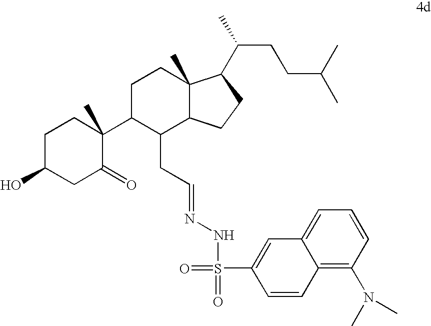

The product of dansyl hydrazine reaction with cholesterol ozonation product 5a was 5c, which is depicted below.

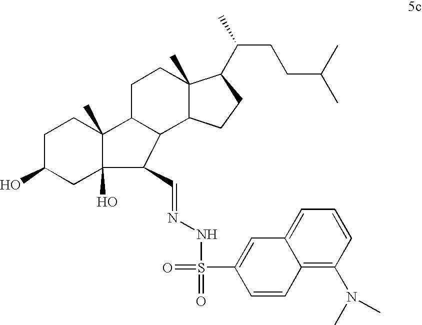

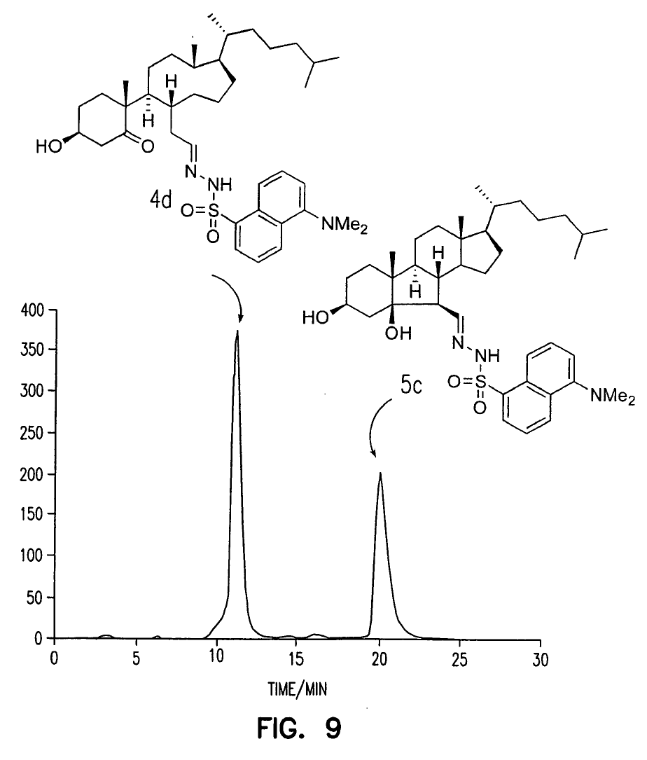

The reaction efficiency for dansyl hydrazine derivatization was evaluated in a range of solvents, such as hexanes, methanol, chloroform, tetrahydrofuran, acetonitrile, and isopropanol (IPA). From this analysis, it was determined that IPA was the optimal solvent in terms of reaction efficiency and lowest rate of spontaneous aldolization of cholesterol ozonation product 4a to 5a. The reaction efficiency was quantified by HPLC using chemically synthesized authentic dansyl hydrazone standards 4d and 5c (FIG. 9). The derivatization efficiency for cholesterol ozonation product 4a with dansyl hydrazine (200 μM) and sulfuric acid (100 μM) in IPA at 37° C. for 48 h, to form 4a hydrazone derivative 4d with a retention time (R_{T}) of about 11.2 min, was 86.0±8.0%. Importantly, only 1.3% of 5c was formed by aldolization of 4a or 4d during the derivatization process. The efficiency of conversion of 5a into its dansyl hydrazone derivative 5c (R_{T}˜19.4 min) was 83±11% for a concentration range of 5a from 0.01-100 μM. The level of sensitivity for the dansyl-hydrazones 4d and 5c is ˜10 nM.

To determine the efficiency by which the 4a and 5a cholesterol ozonation products are extracted and derivatized from plasma samples, human plasma samples were spiked with 5a and then extracted and conjugated with either 2,4-DNP or dansyl hydrazine. There was no significant difference in the amount of conjugated hydrazone detected with either method; 37.5±1.9% derivatized as the dansyl hydrazone 5c and 31±8.9% recovered as 2,4-DNP hydrazone 5b.

Isotope Dilution-gas Chromatography with in-line Mass Spectrometry (ID-GCMS).

At present, most analytical methods for the determination of oxysterols in cholesterol-rich tissues, such as blood (plasma) and atherosclerotic arteries are based on GC with flame ionization detection (FID) or selected ion monitoring (SIM). The advantage of SIM over FID methods is the specificity of detection. This specificity is required for the analysis of oxysterols in biological matrices. The critical aspect to the SIM strategy is the use of internal standards. The most common being 5α-cholestane. See, Jialil, I.; Freeman, D. A.; Grundy, S. M. Aterioscler. Thromb. 1991, 11, 482-488; Hodis, H. N.; Crawford, D. W.; Sevanian, A. Atherosclerosis 1991, 89, 117-126. However, GC-MS with deuterium-labeled internal standards is the preferred method because it is sensitive and specific and corrects for the different recovery of different analytes. Dzeletovic, S.; Brueuer, O.; Lund, E.; Diszfalusy, U. Analytical Biochem. 1995, 225, 73-80. The role of the deuterated internal standards is two-fold. First, they allow quantification by allowing a correlation of isotope abundance with concentration. Second, the addition of a known amount of the deuterated molecule prior to the extraction procedure allows an assessment of the efficiency with which the cholesterol ozonation products are being extracted. Leoni, V.; Masterman, T.; Patel, P.; Meaney, S.; Diczfalusy, U.; Bjørkhelm, I. J. Lipid. Res. 2003, 44, 793-799.

Hexadeuterated cholesterol ozonation products D₆-4a and D₆-5a were prepared from [26, 26, 26, 27, 27, 27-D]-cholesterol (deuterated 3c) as outlined below. 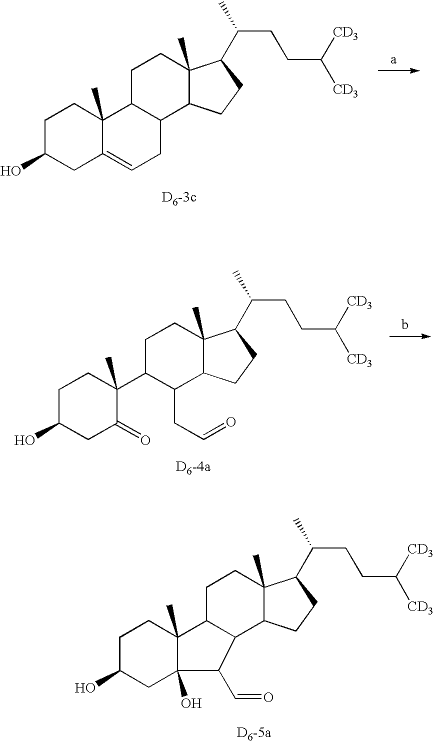
In the first step (a) of the synthesis, ozone was bubbled through a solution of D₆-3c in chloroform-methanol (9:1) at 78° C. to generate D₆-4a. In a second step (b), D₆-4a was dissolved in DMSO and reacted with proline for 2.5 hours at room temperature to generate D₆-5a.

D₆-4a and D₆-5a were used as internal standards to test the sensitivity of the GC/MS method on an in-house Agilent GC/MS. In a typical procedure, samples of authentic cholesterol, 4a, 5a, D₆-cholesterol, D₆-4a and D₆-5a were converted into their trimethylsilylethers by treatment with pyridine and BSTFA under argon at 37° C. for 2 h. After removal of the volatiles (in vacuo) the residue was dissolved in methylene chloride and transferred to an autosampler vial.

GC-MS was then performed on an Agilant Technologies 6890 GC (with a split/splitless inlet system and a 7683 autoinjector module) coupled to a 5973 Inert MSD. The mass spectrometer was operated in the full ion scan mode. The observed retention times (R_{T}) and M⁺ ions were as follows ozonation products 4a and 5a (R_{T}=29.6 min, M⁺ 354); D₆-4a and D₆-5a (R_{T}=29.6 min, M⁺ 360); cholesterol (R_{T}=27.2 min, M⁺ 329), D₆-cholesterol (R_{T}=27.2 min, M⁺ 335). The deduced fragmentation of cholesterol ozonation products 4a and 5a within the GC-MS is shown below. 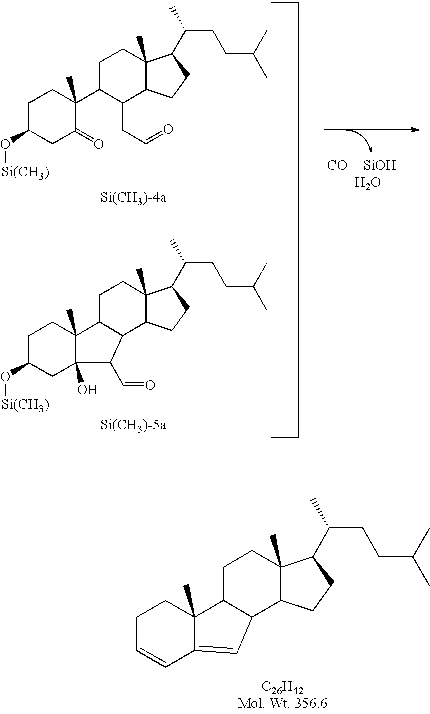
As indicated above, both cholesterol ozonation product 4a and 5a give rise to a fragment of about M+ 354. The deuterated (D₆) 4a and 5a cholesterol ozonation products rise to a fragment of about M+ 360.

Thus, no distinction between cholesterol ozonation products 4a and 5a was observed in the GC-MS assay, probably because cholesterol ozonation product 4a is converted into 5a during the silylation step. Thus, the amount of M+ 354 (or 360) is a measure of the concentration of authentic 4a and 5a cholesterol ozonation product. The area of the 354 ion peak is linear with concentration and the lower-level of sensitivity measured thus far is 10 fg/μL for the cholesterol ozonation products (equivalent to an estimated 2-log increase in detection limit from the LC/MS assay described in previous examples).

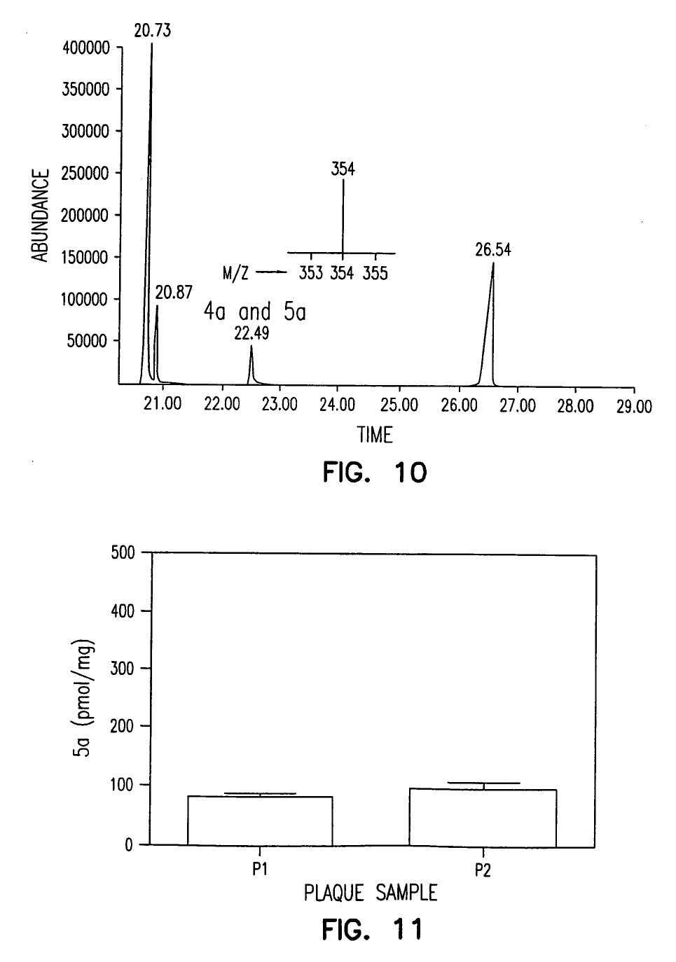

The GCMS assay was further validated by extraction of cholesterol ozonation products from clinically excised carotid plaque material. Carotid endarterectomy tissue (n=2) that had been obtained from patients undergoing carotid endarterectomy for routine analysis were homogenized using a tissue homogenizer for 10 min (under argon) and then extracted into CHCl₃/MeOH. The extract was silylated as described vide supra and then subjected to GC-MS analysis (FIGS. 10 and 11). The GC-MS trace of ion-abundance versus time shows the presence of many oxysterols that have yet to be defined. However, there was clear resolution of the combined ozonation products 4a and 5a (R_{T}=22.49 min).

These data clearly establish the feasibility of the overall extraction and GC-MS assay for the analysis of the 4a and 5a cholesterol ozonation products in biological samples and validate the results described on analysis of atherosclerotic plaque material in previous Examples.

Immunohistochemical Localization of Cholesterol Ozonation Products 4a and 5a.

As described above, mice were immunized with a KLH-conjugate of compound 15a, which is an analog of cholesterol ozonation product 4a. Monoclonal antibodies were generated by hybridoma methods. Two murine monoclonal antibodies, 11C5 and 7A7 with good binding affinity <1 μM for cholesterol ozonation product 5a and excellent specificity over cholesterol (1000 fold less affinity).

Generation of an anti-5a antibody to a hapten that is a 4a analog was not too surprising because, as shown above, addition of cholesterol ozonation product 4a to blood results in its immediate conversion into 5a.

Immunohistochemical staining of frozen fixed sections of aorta from ApoE deficient mice with antibody 11C5 and a FITC-labeled anti IgG secondary antibody demonstrated localization of cholesterol ozonation product 5a in areas of atherosclerosis within subintimal layers of the vessel when compared with consecutive sections stained with non-specific murine antibodies. Absorption of the antibody with soluble cholesterol did not eliminate the subintimal fluorescence.

### REFERENCES

1. P. Wentworth Jr. et al., Science 298, 2195 (2002).
2. B. M. Babior, C. Takeuchi, J. Ruedi, A. Guitierrez, P. Wentworth Jr., Proc. Natl. Acad. Sci. U.S.A. 100, 3920 (2003).
3. P. Wentworth Jr. et al., Proc. Natl. Acad. Sci. U.S.A. 100, 1490 (2003).
4. R. Ross, New Engl. J. Med. 340, 115 (1999).
5. G. K. Hansson, P. Libby, U. Schönbeck, Z.-Q. Yan, Circ. Res. 91, 281 (2002).
6. D. Steinberg, J. Biol. Chem. 272, 20963 (1997).
7. D. Steinberg, S. Parthasarathy, T. E. Carew, J. C. Khoo, J. L. Witztum, New Engl. J. Med. 320, 915 (1989).
8. U. P. Steinbrecher, S. Parthasarathy, D. S. Leake, J. L. Witzum, D. Steinberg, Proc. Natl. Acad. Sci. U.S.A. 81, 3883 (1984).
9. K. Takeuchi, S. Kutsuna, T. Ibusuki, Anal. Chim. Acta 230, 183 (1990).
10. K. Takeuchi, I. Takeuchi, Anal. Chem. 61, 619 (1989).
11. M. J. Steinbeck, A. U. Khan, M. J. Karnovsky, The Journal of Biological Chemistry 267, 13425 (1992).
12. H. Hietter, P. Bischoff, J. P. Beck, G. Ourisson, B. Luu, Cancer Biochem. Biophys. 9, 75 (1986).
13. J. L. Lorenso, M. Allorio, F. Bemini, A. Corsini, R. Fumagalli, FEBS Lett. 218, 77 (1987).
14. D. M. Small, Arteriosclerosis 8, 103 (1988).
15. J. Gumulka, J. St-Pyrek, L. L. Smith, Lipids 17, 197 (1982).
16. J. Gumulka, L. L. Smith, J. Am. Chem. Soc. 105, 1972 (1983).
17. K. Jaworski, L. L. Smith, J. Org. Chem 53, 545 (1988).
18. Z. Paryzek, J. Martynow, W. Swoboda, J. Chem. Soc. Perkin Trans. 1, 1222 (1990).
19. J. W. Comforth, G. D. Hunter, G. Popjak, Biochem. J. 54, 590 (1953).
20. K. Wang, E. Bermúdez, W. A. Pryor, Steroids 58, 225 (1993).
21. E. Lund, I. Björkhem, Acc. Chem. Res. 28, 241 (1995).
22. T. Miyamoto, K. Kodama, Y. Aramaki, R. Higuchi, R. W. M. Van Soest, Tetrahedron Letter 42, 6349 (2001).
23. J. A. Levy, M. Virolainen, V. Defendi, Cancer 22, 517 (1968).
24. A. Weiss, R. L. Wiskocil, J. D. Stobo, J. Immunol. 133, 123 (1984).
25. J. Folkman, C. C. Haudenschild, B. R. Zetter, Proc. Natl. Acad. Sci. U.S.A. 76, 5217 (1979).
26. P. Ralph, M. A. Moore, K. Nilsson, J. Exp. Med. 143, 1528 (1976).
27. I. N. Mbawuike, H. B. Herscowitz, J. Leukoc. Biol. 46, 119 (1989).
28. J. T. N. Hiltermann et al., Free Radical Biology & Medicine 27, 1448 (1999).
29. M. Longphre, L.-Y. Zhang, J. R. Harkema, S. R. Kleeberger, J. Appl. Physiol. 86, 341 (1999).
30. M. T. Krishna et al., Eur. Respir. J. 11, 1294 (1998).
31. Q. Zhao, L. G. Simpson, K. E. Driscoll, G. D. Leikauf, American Journal of Physiology 274, L39 (1998).
32. M. D. Cohen, M. Sisco, Y. Li, J. T. Zelikoff, R. B. Schlesinger, Toxicology and Applied Pharmacology 171, 71 (2001).
33. J. L. Goldstein, Y. K. Ho, S. K. Basu, M. S. Brown, Proc. Natl. Acad. Sci. U.S.A. 76,333 (1979).
34. W. Li, H. Dalen, J. W. Eaton, X.-M. Yuan, Arterioscler. Thromb. Vasc. Biol. 21, 1124 (2001).
35. W. Guo, J. D. Morrisett, M. E. DeBakey, G. M. Lawrie, J. A. Hamilton, Arterioscler. Thromb. Vasc. Biol. 20, 1630 (2000).
36. B. Liu, Z. Weishan, Tetrahedron Lett. 43, 4187 (2002).
37. K. Wang, E. Bermúdez, W. A. Pryor, Steroids 58, 225 (1993).
38. T. Miyamoto, K. Kodama, Y. Aramaki, R. Higuchi, R. W. M. Van Soest, Tetrahedron Letter 42, 6349 (2001).
39. P. Yates, S. Stiveer, Can. J. Chem. 66, 1209 (1988).
40. A. Sevanian, A. R. Peterson, Proc. Natl. Acad. Sci. U.S.A. 81, 4198 (1984).
41. U. P. Steinbrecher, J. L. Wiztum, S. Parthasarathy, D. Steinberg, Arteriosclerosis 1, 135 (1987).
42. L. G. Fong, S. Parthasarathy, J. L. Wiztum, D. Steinberg, J. Lipid. Res. 28, 1466(1987).
43. T. Parasassi et al., Free Radical Biol. & Med. 31, 82 (2001).
44. F. Ursini, K. J. A. Davies, M. Maiorino, T. Parasassi, A. Sevanian, Trends in Mol. Med. 8, 370 (2002).
45. R. Brunelli et al., Biochemistry 39, 13897 (2000).
46. S. Lund-Katz, P. M. Laplaud, M. C. Phillips, M. J. Chapman, Biochemistry 37, 12867 (1998).
47. G. C. Chen et al., J. Biol. Chem. 269, 29121 (1994).
48. E. Lund, I. Björkhem, Acc. Chem. Res. 28, 241 (1995).
49. R. Ross, J. A. Glomset, New Engl. J. Med. 295, 369 (1976).
50. P. Wentworth Jr. et al., Science 293, 1806 (2001).
51. J.-L. Reymond, Y. Chen, J. Org. Chem. 60, 6970 (1995).
52. J. Gumulka, J. St-Pyrek, L. L. Smith, Lipids 17, 197 (1982).
53. P. Wentworth Jr. et al., Science 293, 1806 (2001).

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. An isolated ozonation product of cholesterol that produced in an atherosclerotic plaque.

2. The ozonation product of claim 1 having formula 4a:

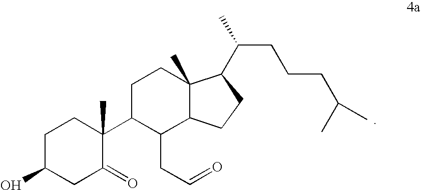

3. The ozonation product of claim 1 having formula 5a:

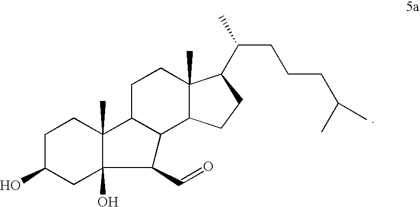

4. The ozonation product of claim 1 having any one of formulae 6a-15a or 7c:

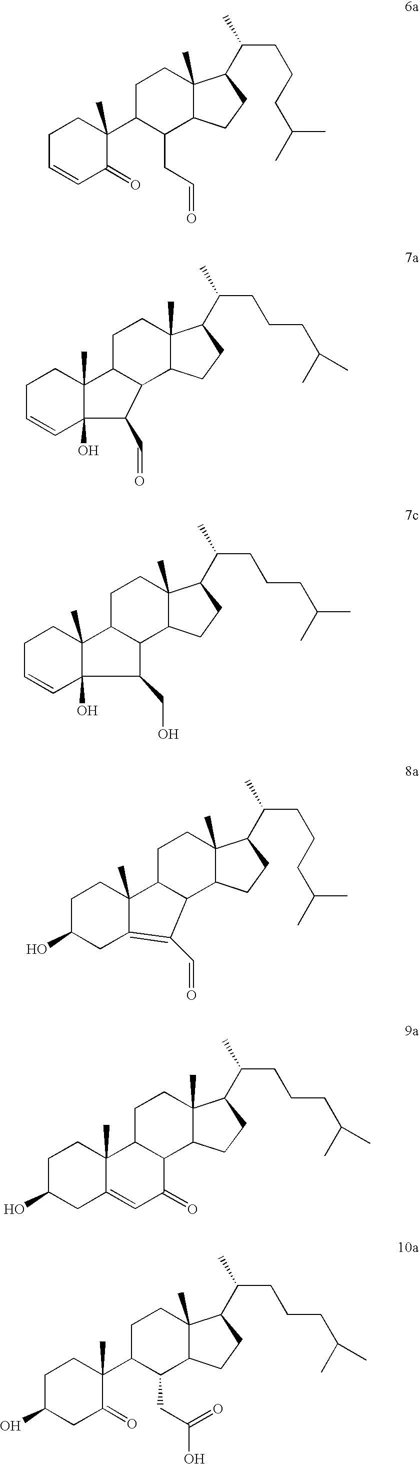

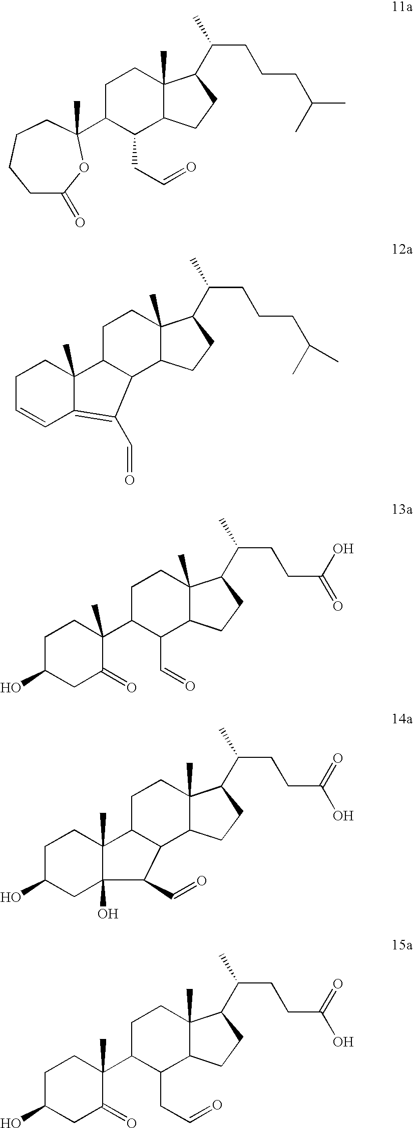

5. A detectable derivative of a cholesterol ozonation product, comprising a bisulfite adduct, an imine, an oxime, a hydrazone, a dansyl hydrazone, a semicarbazone, or a Tollins test product, wherein the ozonation product of cholesterol is generated within an atherosclerotic plaque.

6. A hydrazone derivative of an ozonation product of cholesterol, wherein the ozonation product of cholesterol is generated within an atherosclerotic plaque.

7. The hydrazone derivative of claim 6 having formula 4b or formula 4c:

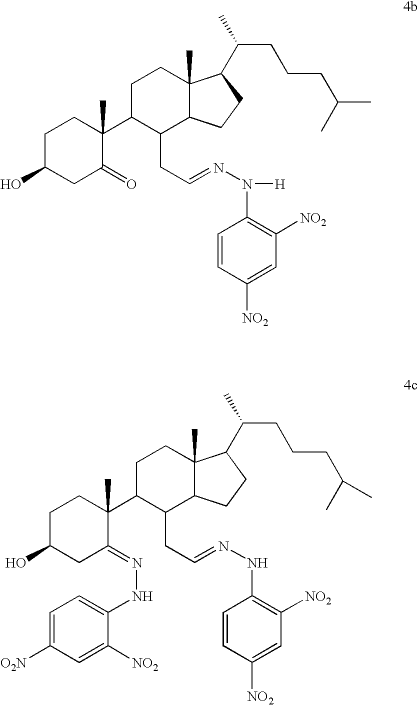

8. The hydrazone derivative of claim 6 having formula 5b:

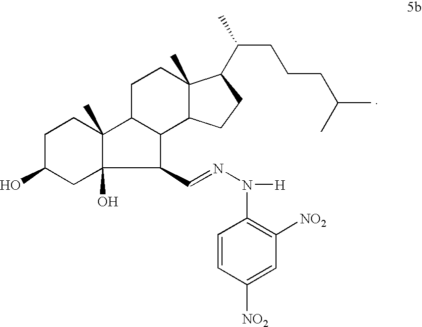

9. The hydrazone derivative of claim 6 any one of formulae 6b-15b or 10c:

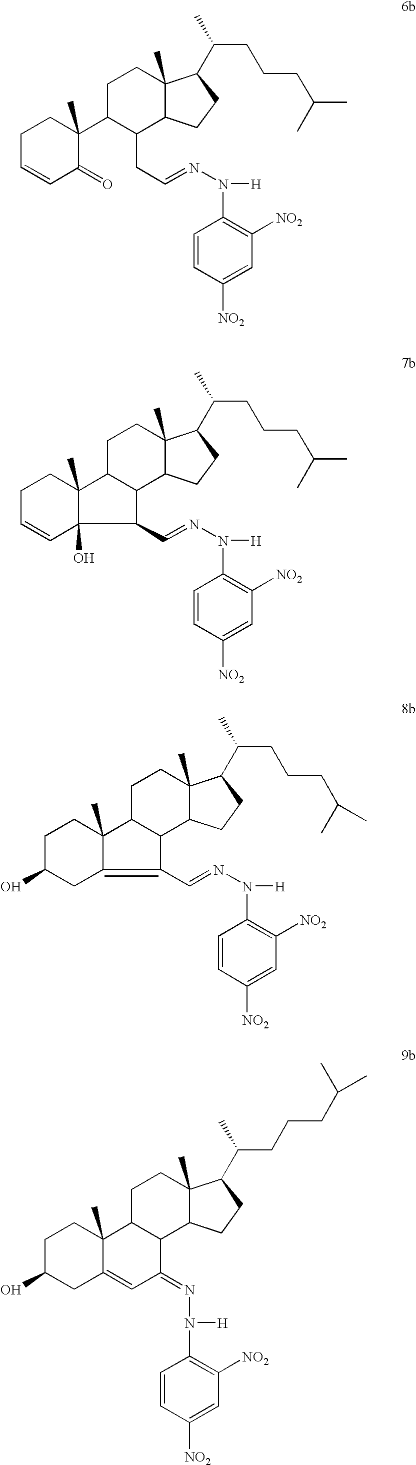

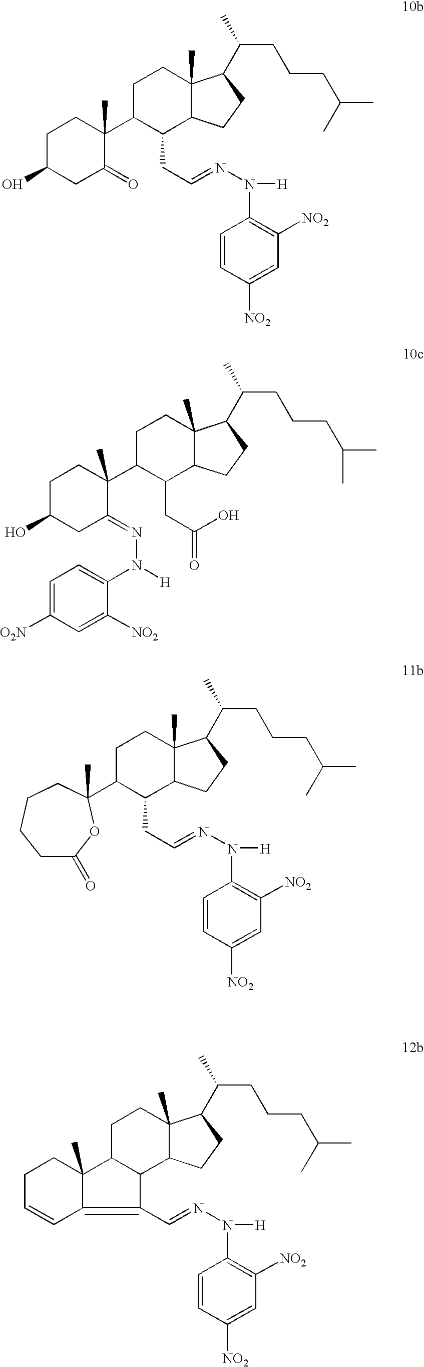

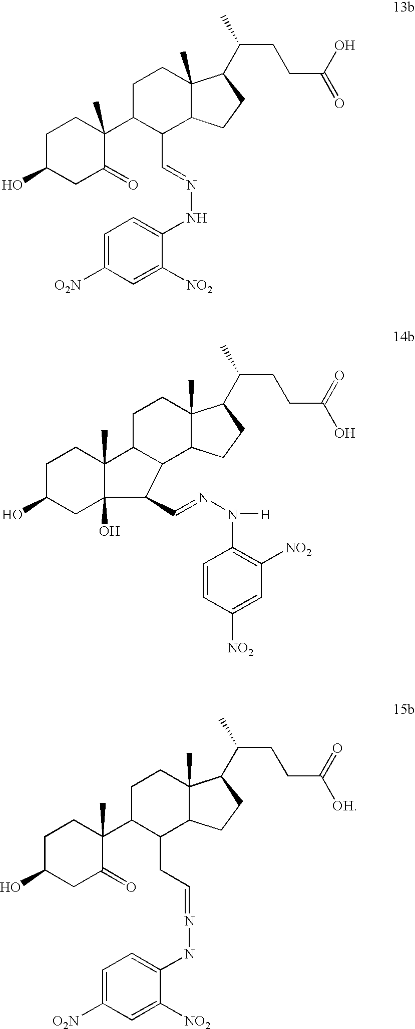

10. A dansyl hydrazone derivative of claim 6 having formula 4d:

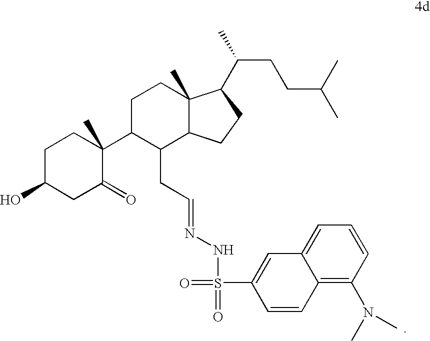

11. A dansyl hydrazone derivative of claim 6 having formula 5c:

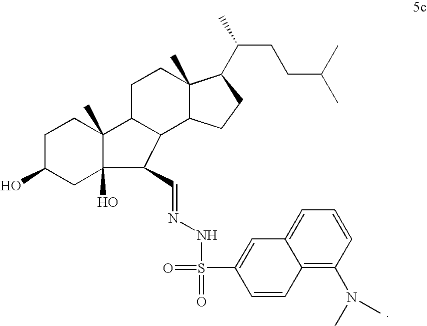

12. A hapten having formula 13a or 13b: 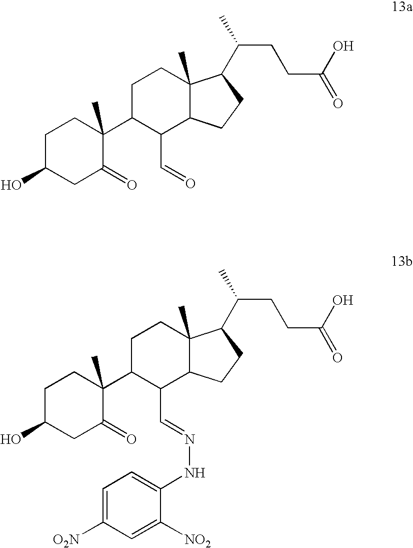
wherein the hapten can be used to generate antibodies that can react with a ozonation or hydrazone product of cholesterol.

13. A hapten having formula 14a or 14b: 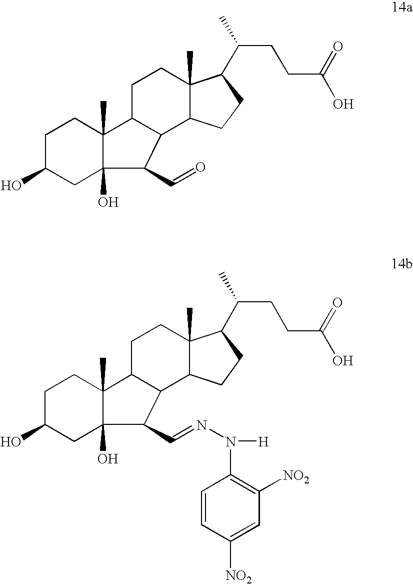
wherein the hapten can be used to generate antibodies that can react with a ozonation or hydrazone product of cholesterol.

14. A hapten having formula 3c: 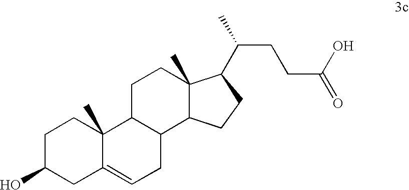
wherein the hapten can be used to generate antibodies that can react with a ozonation or hydrazone product of cholesterol.

15. A hapten having formula 15a: 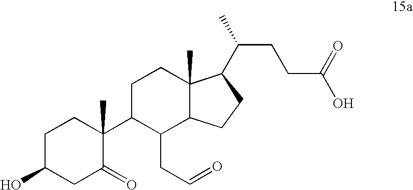
wherein the hapten can be used to generate antibodies that can react with a ozonation or hydrazone product of cholesterol.

16. An isolated antibody that can bind to an ozonation product of cholesterol.

17. A mono clonal antibody that can bind to an ozonation product of cholesterol.

18. The antibody of claim 16 or 17, wherein the ozonation product of cholesterol has formula 4a:

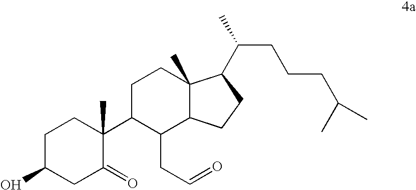

19. The antibody of claim 16 or 17, wherein the ozonation product of cholesterol has formula 5a:

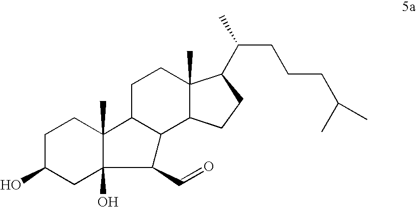

20. The antibody of claim 16 or 17, wherein the ozonation product of cholesterol has any one of formulae 6a-14a, or 7c:

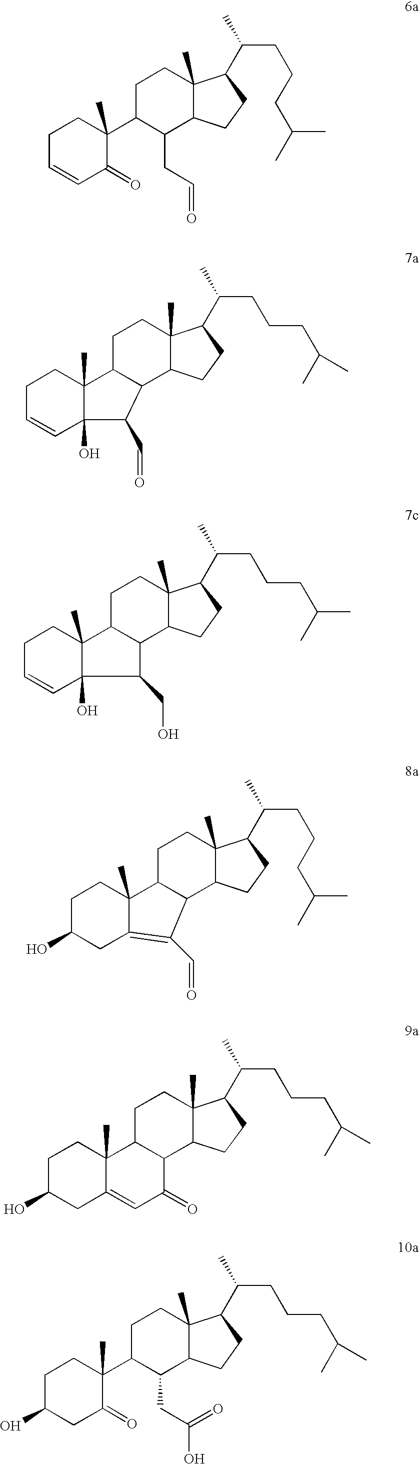

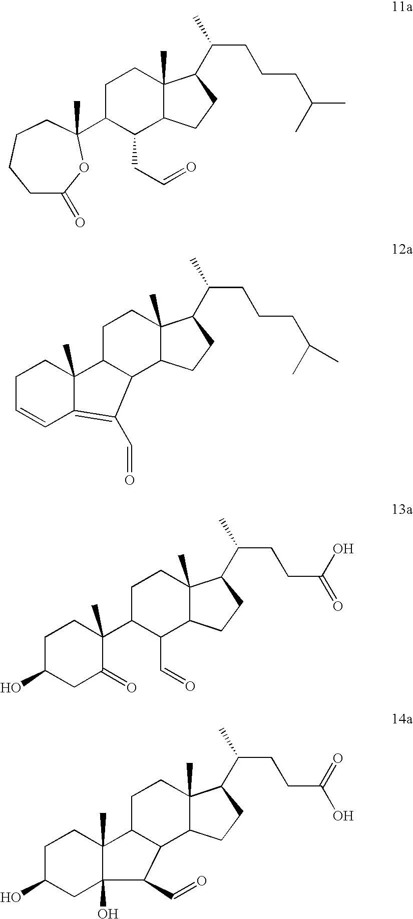

21. The antibody of claim 16 or 17, wherein the antibody was raised against a hapten that has formula 15a:

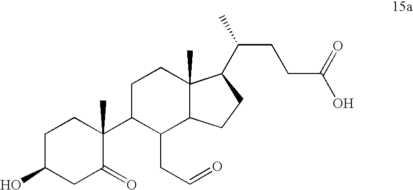

22. An isolated antibody that can bind to a hydrazone derivative of an ozonation product of cholesterol.

23. The isolated antibody of claim 22, wherein the hydrazone derivative has formula 4b or formula 4c:

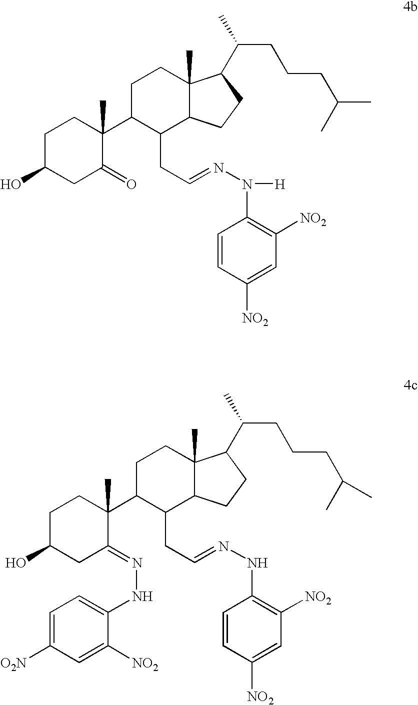

24. The isolated antibody of claim 22, wherein the hydrazone derivative has formula 5b:

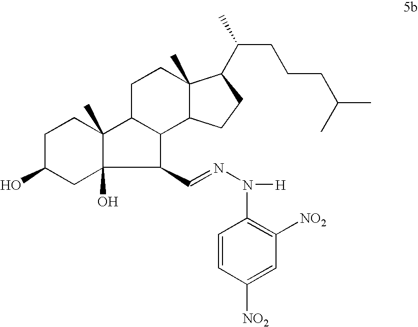

25. The isolated antibody of claim 22, wherein the hydrazone derivative has any one of formulae 6b-15b or 10c:

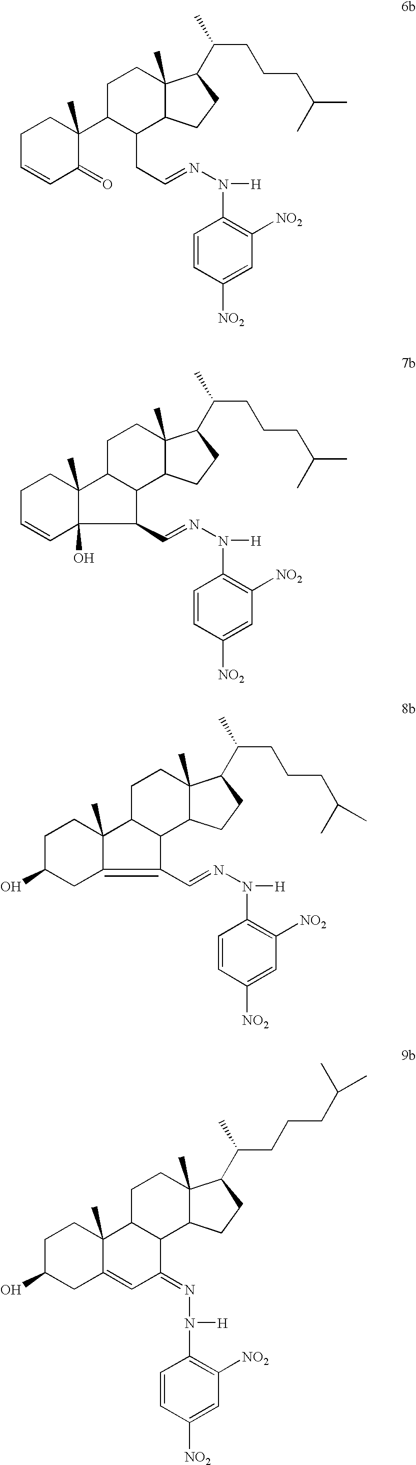

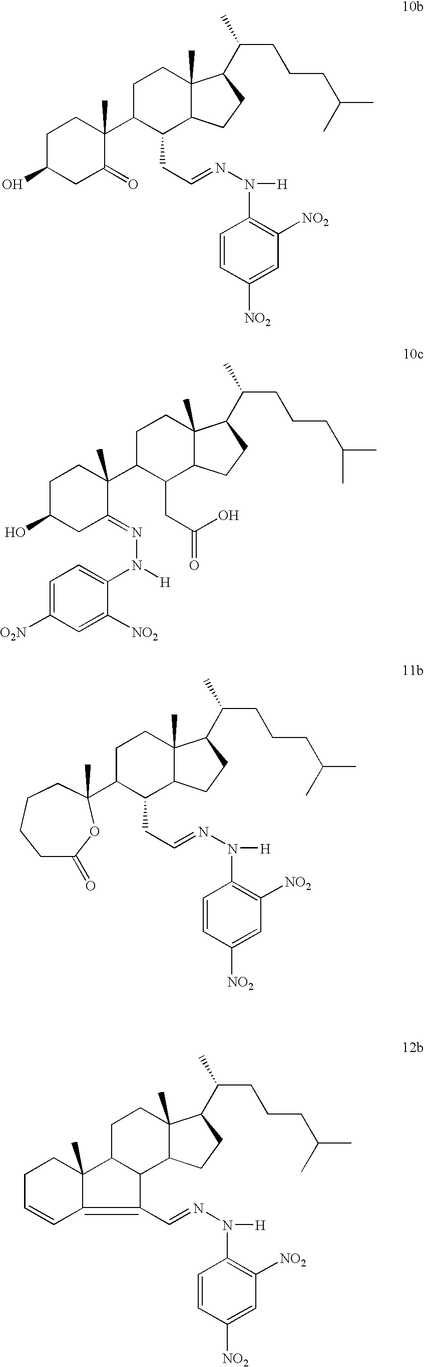

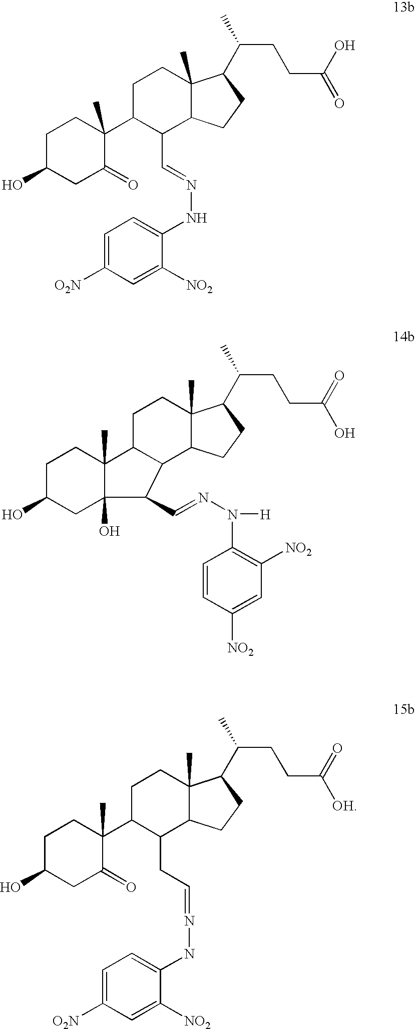

26. The isolated antibody of claim 22, wherein the isolated antibody is raised against a hapten having formula 13a or 14a:

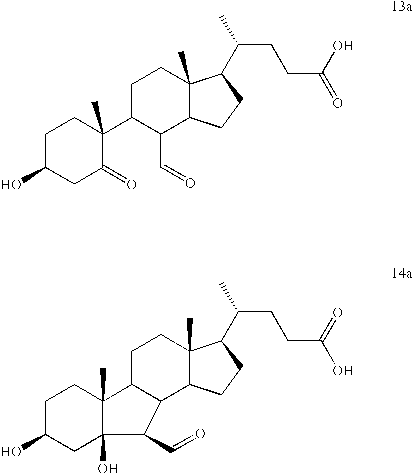

27. The isolated antibody of claim 22, wherein the isolated antibody is raised against a hapten having formula 15a:

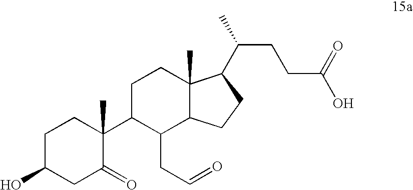

28. An isolated antibody, wherein the isolated antibody is a derived from hybridoma KA1-11C5:6 or KA1-7A6:6 having ATCC Accession No. PTA-5427 or PTA-5428.

29. An isolated antibody, wherein the isolated antibody is a derived from hybridoma KA2-8F6:4 or KA2-1E9:4, having ATCC Accession No. PTA-5429 and PTA-5430.

30. A method for detecting atherosclerosis in a patient comprising: detecting whether an ozonation product of cholesterol is present in the test sample obtained from a patient.

31. The method of claim 30, wherein the ozonation product is generated by an atherosclerotic plaque.

32. The method of claim 30, wherein the test sample is serum, plasma, blood, atherosclerotic plaque material, urine or vascular tissue.

33. The method of claim 30, wherein the ozonation product is a compound having formula 4a:

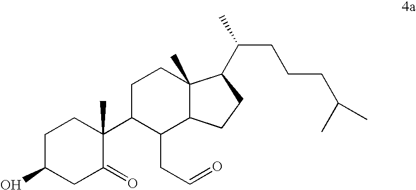

34. The method of claim 30, wherein the ozonation product is a compound having formula 5a:

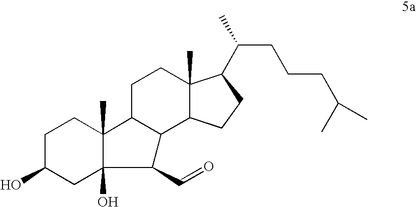

35. The method of claim 30, wherein the ozonation product is a compound having any one of formulae 6a-15a, or 7c:

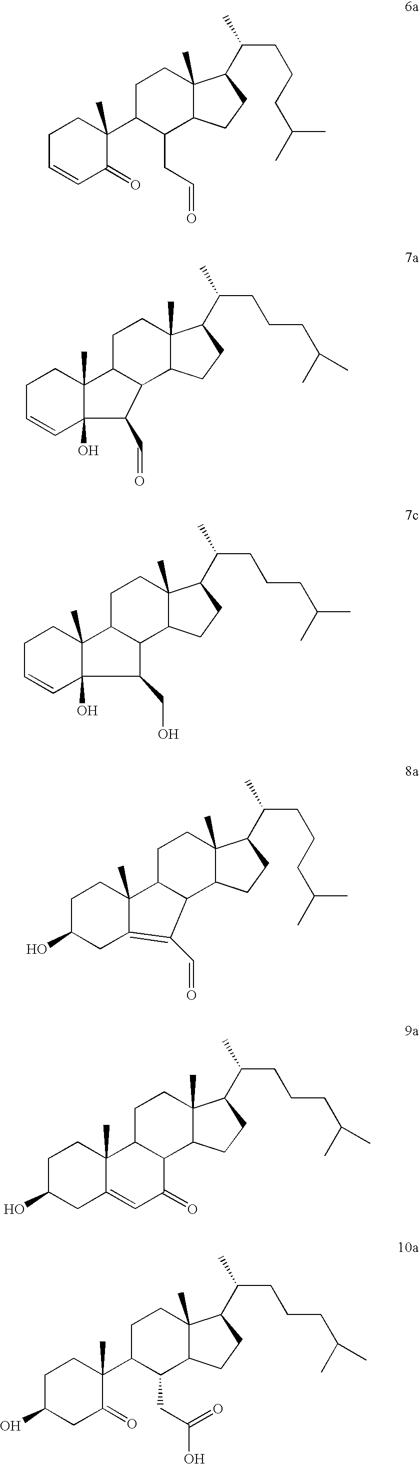

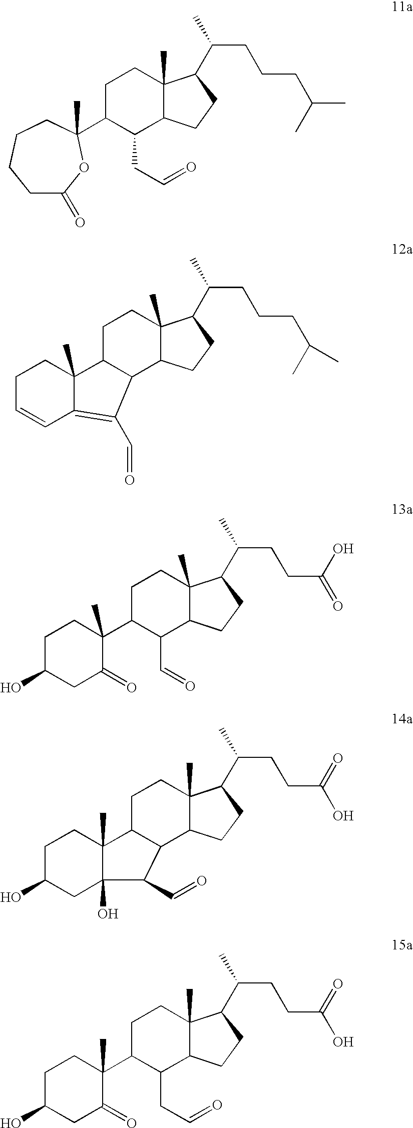

36. The method of claim 30, wherein the method further comprises reacting the test sample with a bisulfite, ammonia, Schiff's base, aromatic or aliphatic hydrazines, dansyl hydrazine, Gerard's reagent, Tollins test reagent and detecting a derivative of an ozonation product of cholesterol that is formed by such reaction.

37. The method of claim 30, wherein the method further comprises reacting the test sample with a hydrazine compound to generate a hydrazone derivative of an ozonation product of cholesterol.

38. The method of claim 37, wherein the hydrazine compound is 2,4-dinitrophenyl hydrazine.

39. The method of claim 37, wherein the hydrazone derivative has formula 4b or formula 4c:

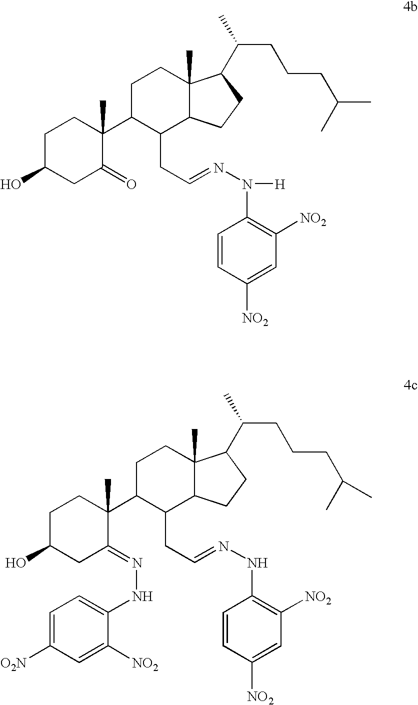

40. The method of claim 37, wherein the hydrazone derivative has formula 5b:

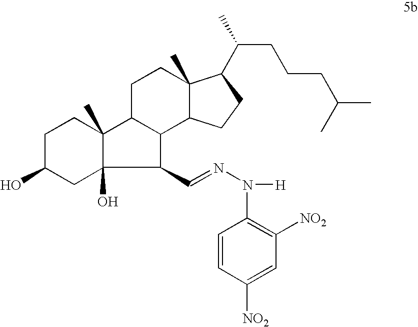

41. The method of claim 37, wherein the hydrazone derivative has any one of formulae 6b-15b or 10c:

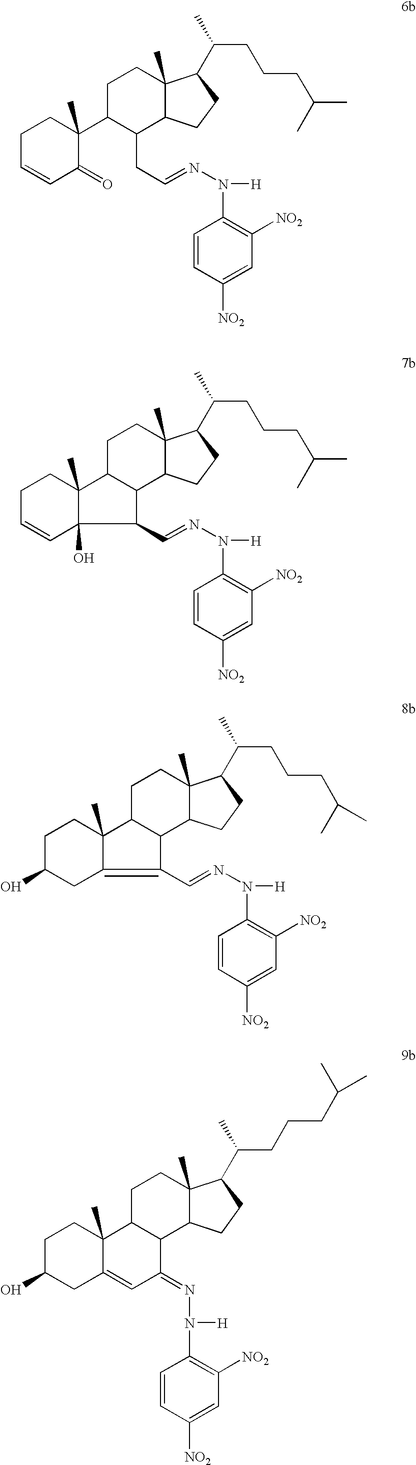

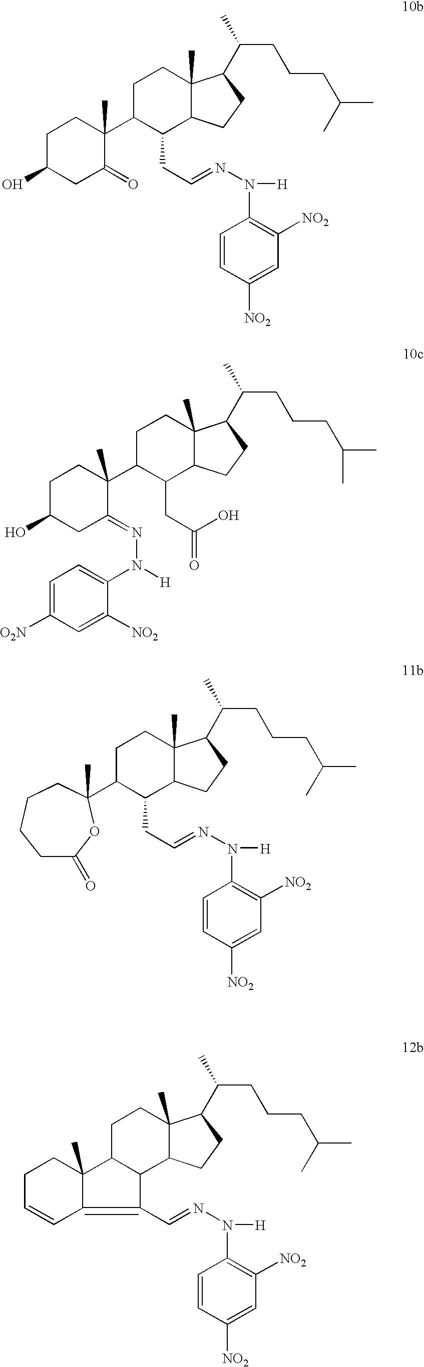

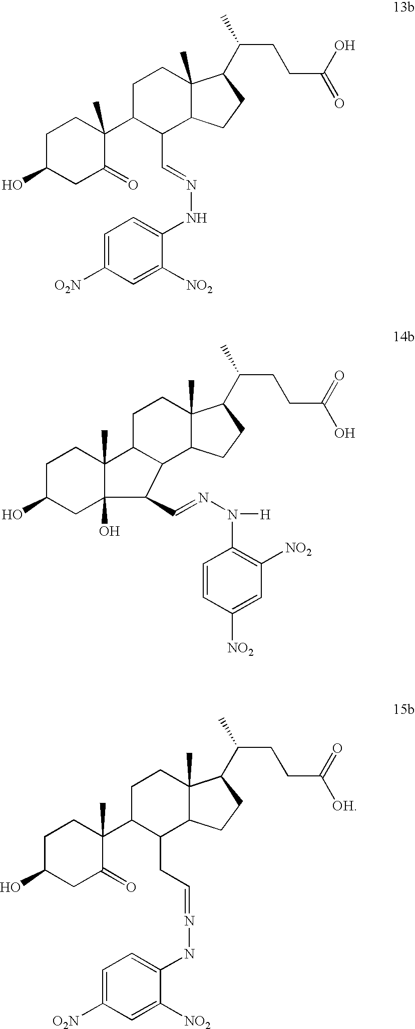

42. The method of claim 30, wherein the method further comprises reacting the test sample with dansyl hydrazine to generate a dansyl hydrazone derivative of an ozonation product of cholesterol.

43. The method of claim 42, wherein the dansyl hydrazone derivative has formula 4d or 5c:

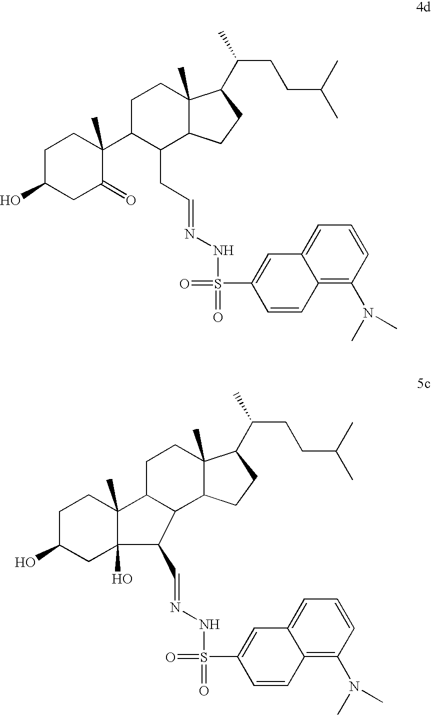

44. The method of claim 30, wherein the method further involves contacting the test sample with an antibody that can bind to an ozonation product of cholesterol.

45. The method of claim 44, wherein the antibody is raised against a hapten having formula 13a or 14a:

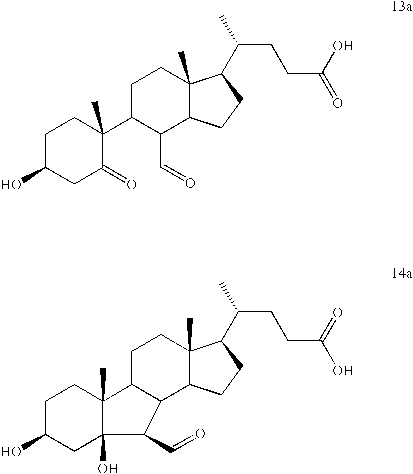

46. The method of claim 44, wherein the antibody is raised against a hapten having formula 15a:

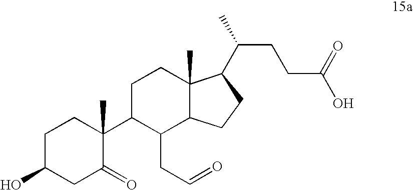

47. The method of claim 44, wherein the antibody is derived from hybridoma KA1-11C5:6 or KA1-7A6:6 having ATCC Accession No. PTA-5427 or PTA-5428.

48. The method of claim 44, wherein the antibody is derived from hybridoma KA2-8F6:4 or KA2-1E9:4, having ATCC Accession No. PTA-5429 and PTA-5430.

49. The method of claim 44, wherein the antibody can bind to a compound having formula 4a:

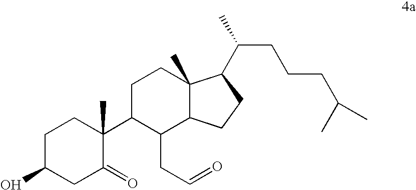

50. The method of claim 44, wherein the antibody can bind to a compound having formula 5a:

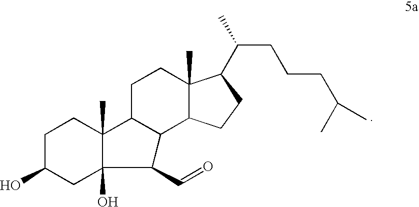

51. The method of claim 44, wherein the antibody can bind to a compound having any one of formulae 6a-15a, or 7c:

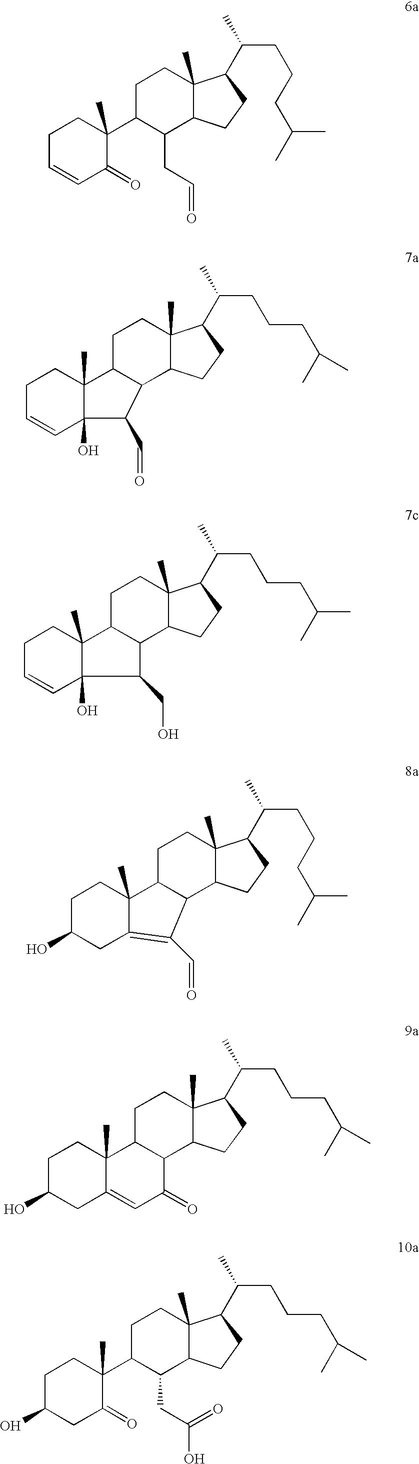

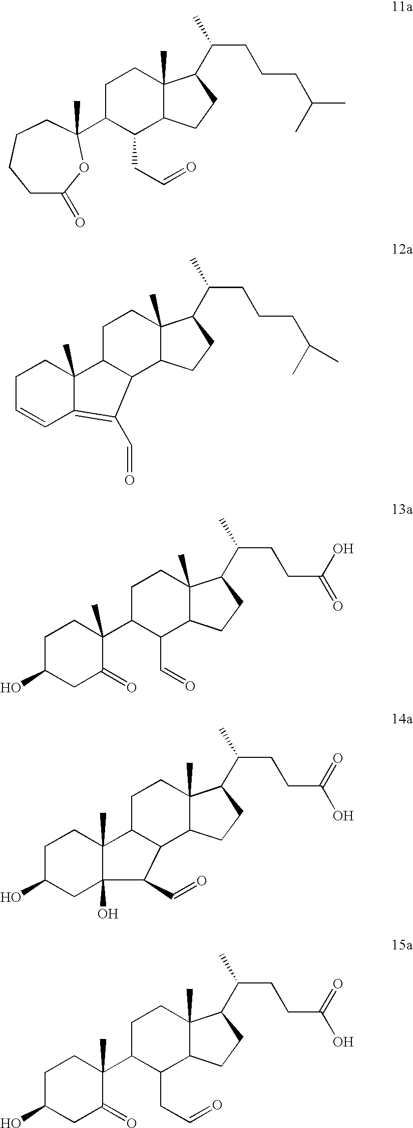

52. A method for detecting whether an ozonation product of cholesterol is released by an atherosclerotic plaque in a patient comprising: detecting whether an ozonation product of cholesterol is present in a test sample obtained from a patient, wherein the ozonation product is a compound comprising formula 5a:

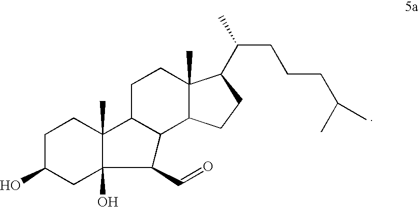

53. A method for detecting atherosclerosis in a patient comprising: adding 2,4-dinitrophenylhydrazine to a test sample from the patient and detecting whether a hydrazone derivative of an ozonation product of cholesterol is present in the test sample.

54. The method of claim 53, wherein the hydrazone derivative has formula 4b, 4c, 5b, 6b, 7b, 8b, 9b, 10b, 10c, 11b, 12b, 13b, 14b or 15b:

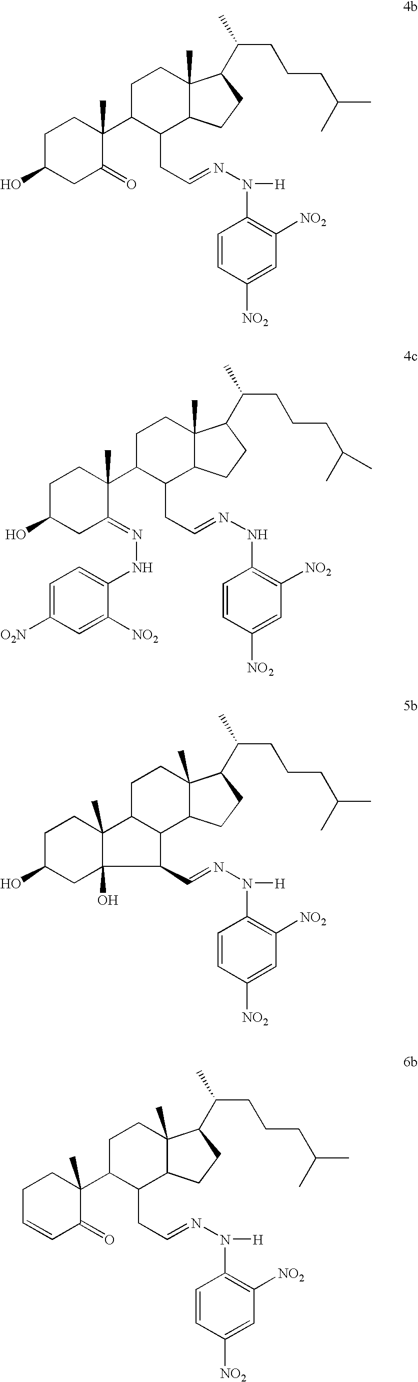

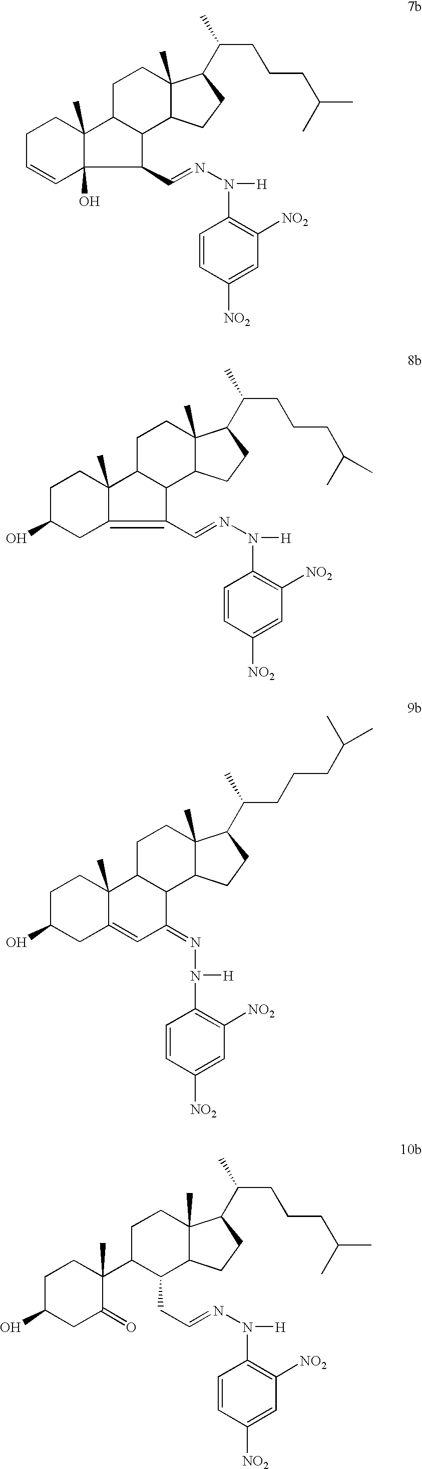

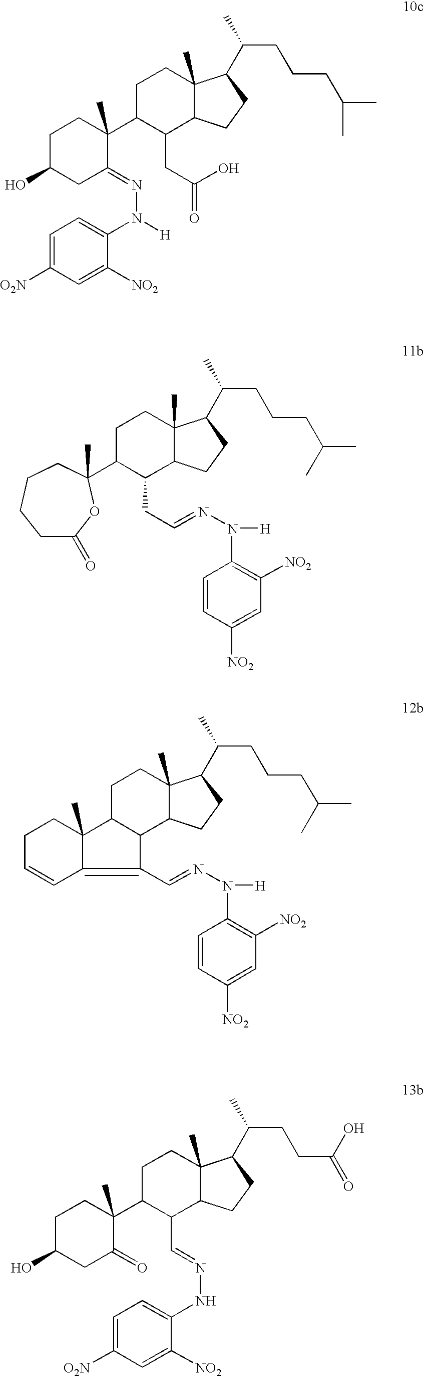

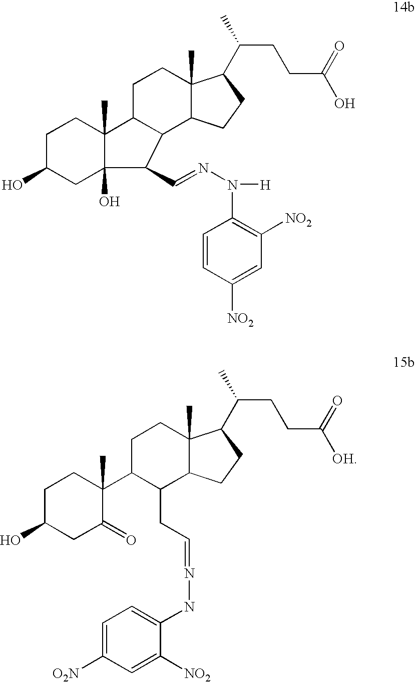

55. A method for detecting atherosclerosis in a patient comprising: adding dansyl hydrazine to a test sample from the patient and detecting whether a dansyl hydrazone derivative of an ozonation product of cholesterol is present in the test sample.

56. The method of claim 55, wherein the dansyl hydrazone derivative is a compound having formula 4d or 5c:

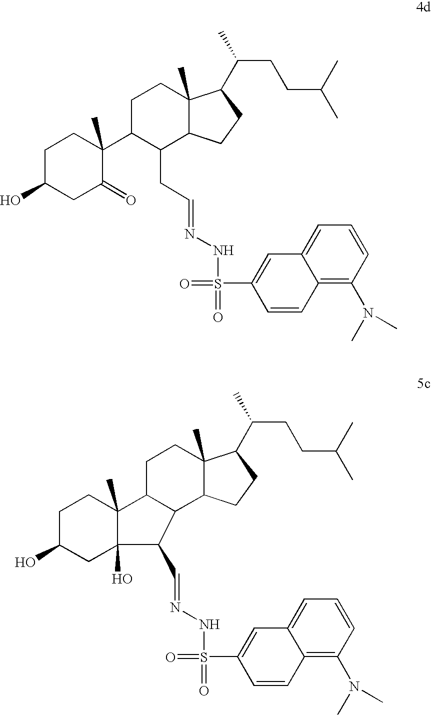

57. A method for detecting whether cholesterol ozonolysis products are present in a test sample comprising contacting macrophages with the test sample and determining whether lipid uptake by macrophages is increased.

58. A method for detecting atherosclerosis in a patient comprising contacting macrophages with a test sample from the patient and determining whether lipid uptake by macrophages is increased.

59. A method for detecting cholesterol ozonolysis products in a test sample comprising contacting low density lipoproteins with the test sample and observing whether the secondary structure of the low density lipoproteins changes.

60. A method for detecting atherosclerosis in a patient comprising contacting low density lipoproteins with a test sample obtained from the patient and observing whether the secondary structure of the low density lipoproteins changes.

61. A method for detecting cholesterol ozonolysis products in a test sample comprising contacting apoprotein B₁₀₀ with the test sample and observing whether the secondary structure of the apoprotein B₁₀₀ changes.

62. A method for detecting atherosclerosis in a patient comprising contacting apoprotein B₁₀₀ with a test sample obtained from the patient and observing whether the secondary structure of the apoprotein B₁₀₀ changes.

63. The method of any one of claims 57-62, wherein the secondary structure of low density lipoproteins or apoprotein B₁₀₀ is observed by circular dichroism.
